SEQUENCE LISTING (26 sequences)

SEQ ID NO: 1 - DNA, 19 bp, Artificial Sequence
  Synthetic oligonucleotide
tgcctgactccttgggacc

SEQ ID NO: 2 - DNA, 22 bp, Artificial Sequence
  Synthetic oligonucleotide
tagtgagcttcttcctcaagca

SEQ ID NO: 3 - DNA, 20 bp, Artificial Sequence
  Synthetic oligonucleotide
aaaccagccaaggtaagcct

SEQ ID NO: 4 - DNA, 20 bp, Artificial Sequence
  Synthetic oligonucleotide
tcagtcactttcaccgggag

SEQ ID NO: 5 - DNA, 24 bp, Artificial Sequence
  Synthetic oligonucleotide
ggtaactctgtctttctaacctta

SEQ ID NO: 6 - DNA, 29 bp, Artificial Sequence
  Synthetic oligonucleotide
gtgatgaccccagcatgcaccagtcacag

SEQ ID NO: 7 - DNA, 19 bp, Artificial Sequence
  Synthetic oligonucleotide
gggctcaaggttctgctgc

SEQ ID NO: 8 - DNA, 20 bp, Artificial Sequence
  Synthetic oligonucleotide
tgggtgtccagcatttcctc

SEQ ID NO: 9 - DNA, 18 bp, Artificial Sequence
  Synthetic oligonucleotide
cagcagcccaaaatgcct

SEQ ID NO: 10 - DNA, 20 bp, Artificial Sequence
  Synthetic oligonucleotide
ttttgagccaggacagctga

SEQ ID NO: 11 - DNA, 19 bp, Artificial Sequence
  Synthetic oligonucleotide
cttgaaccagcagggtggc

SEQ ID NO: 12 - DNA, 20 bp, Artificial Sequence
  Synthetic oligonucleotide
gaggctttggtgtgaagggt

SEQ ID NO: 13 - DNA, 20 bp, Artificial Sequence
  Synthetic oligonucleotide
gacgaagagtgctgtcccaa

SEQ ID NO: 14 - DNA, 21 bp, Artificial Sequence
  Synthetic oligonucleotide
gccaaggggtacatactggag

SEQ ID NO: 15 - DNA, 21 bp, Artificial Sequence
  Synthetic oligonucleotide
atctttagcctcttggcggtg

SEQ ID NO: 16 - DNA, 21 bp, Artificial Sequence
  Synthetic oligonucleotide
gacccagaggacagcaatgat

SEQ ID NO: 17 - DNA, 22 bp, Artificial Sequence
  Synthetic oligonucleotide
accagagcgaaagcatttgcca

SEQ ID NO: 18 - DNA, 22 bp, Artificial Sequence
  Synthetic oligonucleotide
atcgccagtcggcatcgtttat

SEQ ID NO: 19 - DNA, 20 bp, Artificial Sequence
  Synthetic oligonucleotide
tgagagccctcacactcctc

SEQ ID NO: 20 - DNA, 20 bp, Artificial Sequence
  Synthetic oligonucleotide
acctttgctggactctgcac

SEQ ID NO: 21 - DNA, 22 bp, Artificial Sequence
  Synthetic oligonucleotide
aattgcagtgatttgcttttgc

SEQ ID NO: 22 - DNA, 24 bp, Artificial Sequence
  Synthetic oligonucleotide
cagaacttccagaatcagcctgtt

SEQ ID NO: 23 - DNA, 18 bp, Artificial Sequence
  Synthetic oligonucleotide
catctgcctggctccttg

SEQ ID NO: 24 - DNA, 18 bp, Artificial Sequence
  Synthetic oligonucleotide
caggggactggagccata

SEQ ID NO: 25 - DNA, 25 bp, Artificial Sequence
  Synthetic oligonucleotide
ccctgtaattggaatgagtccactt

SEQ ID NO: 26 - DNA, 22 bp, Artificial Sequence
  Synthetic oligonucleotide
acgctattggagctggaattac

(19) United States
(12) Patent Application Publication
(10) Pub. No.: US 2022/0249533 A1
(43) Pub. Date: Aug. 11, 2022

(54) COMPOSITIONS AND METHODS FOR BONE REPAIR AND BONE HEALTH

(71) Applicant: DUKE UNIVERSITY, Durham, NC (US)

(72) Inventors: Shyni Varghese, Durham, NC (US); Yu Ru Shih, Durham, NC (US); Jiaul Hoque, Durham, NC (US)

(73) Assignee: DUKE UNIVERSITY, Durham, NC (US)

(21) Appl. No.: 17/616,499

(22) PCT Filed: Jun. 3, 2020
(86) PCT No.: PCT/US2020/036012

§ 371 Date: Dec. 3, 2021

Related U.S. Application Data
(60) Provisional application No. 62/856,239, filed on Jun. 3, 2019.

(51) Int. Cl. A61K 31/7076; A61K 47/36; A61K 31/69; A61K 31/663; A61K 9/06; A61K 9/00; A61P 19/08

(52) U.S. Cl. CPC A61K 31/7076; A61K 47/36; A61K 31/69; A61P 19/08; A61K 9/06; A61K 9/0019; A61K 31/663

(57) ABSTRACT

The present disclosure relates to polymer-based biomaterials for the systemic or localized delivery of osteoanabolic molecules, and their use in methods for treating and/or preventing bone degeneration and for promoting bone regeneration. In one aspect, the present invention provides a biomaterial comprising a polymer and a bioactive molecule binding moiety. In certain embodiments of the first aspect of the invention, the bioactive molecule binding moiety is an osteoanabolic molecule binding moiety.

### CROSS REFERENCE TO RELATED APPLICATIONS

This application claims priority to U.S. Provisional Patent Application Ser. No. 62/856,239, filed Jun. 3, 2019, the contents of which are hereby incorporated by reference in their entirety.

### STATEMENT REGARDING FEDERALLY SPONSORED RESEARCH OR DEVELOPMENT

This application was made with United States government support under Federal Grant Nos. AR063183 and AR071552 awarded by the NIH/NIAMS. The United States government has certain rights in this invention.

### BACKGROUND OF THE INVENTION

### Field of the Invention

The present disclosure relates to polymer-based biomaterials for the systemic or localized delivery of osteoanabolic molecules, and their use in methods for treating and/or preventing bone degeneration and for promoting bone regeneration.

### Description of the Related Art

A leading concept in regenerative medicine is transplantation of tissue-specific cells, often supported with biomaterials, to promote tissue repair. While this strategy has achieved some success, its broad clinical application is hindered by various challenges such as high costs, constraints associated with cell isolation and expansion, and limited in vivo engraftment of transplanted cells. Instead, harnessing endogenous cells and native biomolecules to augment the innate regenerative ability of tissues has been explored as an alternative. Given that the function of endogenous cells is regulated by their microenvironment, potential of biomaterials and/or growth factors to promote tissue regeneration has been explored extensively. Small molecules are equally powerful in regulating various cellular functions including tissue-specific differentiation of stem cells. Although significant strides have been made in employing small molecules to direct cellular functions in vitro, harnessing small molecules towards tissue repair in vivo still remains limited.

The treatment and/or prevention of bone diseases involving bone degeneration may benefit from such a therapeutic approach. Such diseases include, e.g., osteoporosis and bone fracture. Notably, extracellular adenosine has been shown to play a key role in maintaining bone health and could potentially be used to treat bone loss. However, systemic administration of exogenous adenosine to treat bone disorders is challenging given the ubiquitous presence of adenosine receptors in different organs, the potential for off-target effects associated with its systemic administration, and the short half-life of adenosine in circulation.

Accordingly, there exists a need in the art for therapeutic compounds and methods for the treatment and/or prevention of bone diseases that overcome these challenges.

### SUMMARY OF THE INVENTION

In one aspect, the present invention provides a biomaterial comprising a polymer and a bioactive molecule binding moiety. In certain embodiments of the first aspect of the invention, the bioactive molecule binding moiety is an osteoanabolic molecule binding moiety. In other embodiments of the first aspect of the invention, the biomaterial further comprises a bone targeting moiety. In a further embodiment of the first aspect of the invention, the biomaterial further comprises a bioactive molecule, and in yet a further embodiment, the bioactive molecule is an osteoanabolic molecule.

In a second aspect, the present invention provides a pharmaceutical composition comprising the biomaterials of the invention and a pharmaceutically acceptable carrier and/or excipient.

In a third aspect, the present invention provides a method of reducing bone degeneration and/or promoting bone regeneration in a subject in need thereof comprising administering to the subject a biomaterial of the invention.

In a fourth aspect, the present invention provides a method of promoting osteoblastogenesis and/or decreasing osteoclastogenesis in a subject in need thereof comprising administering to the subject a biomaterial of the invention.

In a fifth aspect, the present invention provides a method treating and/or preventing a low bone mass condition in a subject in need thereof comprising administering to the subject a biomaterial of the invention. In one embodiment of the fifth aspect of the invention, the low bone mass condition is osteoporosis.

In a sixth aspect, the present invention provides a method of promoting bone fracture healing in a subject in need thereof comprising administering to the subject a biomaterial of the invention.

In a seventh aspect, the present invention provides a method of repairing a skeletal defect in a subject in need thereof comprising administering to the subject a biomaterial of the invention.

In an eighth aspect, the present invention provides a method of enhancing the innate ability of bone repair tissue to repair bone in a subject in need thereof comprising administering to the subject a biomaterial of the invention.

In a ninth aspect, the present invention provides a method of activating A2BR to promote bone repair in a subject in need thereof comprising administering to the subject a biomaterial of the invention.

In a tenth aspect, the present invention provides a method of enhancing the outcome of orthopedic implant surgery in a subject in need thereof comprising administering to the subject a biomaterial of the invention.

### BRIEF DESRIPTION OF THE DRAWINGS

FIG. 1. Synthesis of HA-MA or HA-MA-Aln from hyaluronic acid (HA).

FIGS. 2A-2B. Synthesis of cyanine 7.0 conjugated polymers: (2A) Synthesis of HA-MA-Cy7 from HA-MA; (2B) Synthesis of HA-MA-Aln-Cy7 from HA-MA-Aln.

FIGS. 3A-3D. Synthesis, characterization, and adenosine loading/release profile of the nanocarriers. (3A) Structure of the nanocarrier precursor polymer HA-MA-Aln. Chemical structure of (3B) 3-APBA and (3C) adenosine. (3D) Schematic representation of alendronate conjugated nanocarrier (Aln-NC).

FIGS. 4A-4C. Characterization of the modified-Has. (4A) FTIR spectra of the HA-MA, HA-MA-Aln and HA-MA-Aln-Cy7 polymers. Full spectrum from 4000-650 cm⁻¹ (Left image). Extended spectrum from 2000-1000 cm⁻¹ (Right image). Arrows indicate the presence of ester C═O peaks in methacrylated polymers. (4B) ¹HNMR spectrum of HA-MA recorded at 400 MHz in D₂O at 25° C. (4C) ¹HNMR spectrum of HA-MA-Aln recorded at 400 MHz in D₂O at 25° C.

FIGS. 5A-5B. Characterization of nanocarriers. (5A) UV-visible absorbance spectra of the NC and Aln-NC. (5B) Size distribution profile of the nanocarriers.

FIGS. 6A-6C. Characterization of cyanine 7.0 conjugated polymers. (6A) UV-visible absorbance spectra of the cyanine conjugated polymers. Conjugated cyanine shows similar absorbance as non-conjugated cyanine dye. Arrow indicates absorbance peaks for the cyanine 7.0 (at 747-750 nm). (6B) ¹HNMIR spectrum of HA-MA-Aln-Cy7 recorded at 400 MHz in D₂O at 25° C. (6C) UV-visible absorbance spectrum of the cyanine tagged nanocarriers (NC-Cy7 and Aln-NC-Cy7). Conjugated cyanine shows similar absorbance as pure cyanine dye (indicative of no structural changes during the nanocarrier preparation). Arrow indicates absorbance peaks for the cyanine 7.0 (at 747-750 nm).

FIGS. 7A-7B. Adenosine loading. (7A) Reaction scheme showing the reversible adenosine binding with PBA. (7B) Cumulative release kinetics of adenosine from the nanocarriers. Scale bar 200 nm.

FIGS. 8A-8B. Biodistribution of the nanocarriers. (8A) Binding affinity of alendronate-functionalized nanocarrier (Aln-NC) and non-functionalized NC (NC) to femur bone chips: radiant efficiency was expressed by unit surface area of the bone chips (*P<0.0013). (8B) Characterization of the host at 72 h after systemic administration of the nanocarriers in nude mice through tail vein injection; radiant efficiency in organs ex vivo represented per gram of the tissue.

FIGS. 9A-9F. Adenosine encapsulated nanocarrier attenuates bone loss in ovariectomized mice. Administration of Aln-NC containing adenosine (OHA) and Aln-NC without adenosine (OH) in OVX mice for 8 weeks. Groups were compared to healthy control with no surgery and no treatment (CTL) and OVX mice with no treatment (group O). Quantification of μ-CT images of vertebrae: (9A) bone mineral density (BMD); (9B) bone volume (BV/TV); (9C) trabecular number (Tb.N); (9D) trabecular spacing (Tb. Sp); (9E) connectivity density (Conn. D) (9F) trabecular thickness (Tb. Th). *p<0.05, **p<0.01, ***p<0.001.

FIGS. 10A-10F. Adenosine encapsulated nanocarriers attenuate bone loss in OVX mice. Quantification of μCT images of femur: (10A) bone mineral density (BMD); (10B) bone volume (BV/TV); (10C) trabecular number (Tb.N); (10D) trabecular spacing (Tb. Sp); (10E) connectivity density (Conn. D) (10F) trabecular thickness (Tb. Th). *p<0.05, **p<0.01, ***p<0.001.

FIGS. 11A-11B. Adenosine encapsulated nanocarrier promotes bone formation in ovariectomized mice. Administration of Aln-NC containing adenosine (OHA) and carrier Aln-NC alone (OH) for 8 weeks in OVX mice. Groups are compared to healthy control (CTL) and ovariectomized animals (O). (11A) Quantification of bone formation rate (BFR/BS) from bone labeling images. n.d: non-detectable. (11B) Quantification of mineral apposition rate (MAR) from bone labeling images. n.s: no separation. *p<0.05, **p<0.01, ***p<0.001.

FIGS. 12A-12B. Mechanical measurement of tibia following 8 weeks of treatment (12A) maximum load and (12B) stiffness *p<0.05, **p<0.01. CTL: healthy control with no surgery and no treatment. O: ovariectomized animals with no treatment. OH: ovariectomized animals treated with Aln-NC. OHA: ovariectomized animals treated with Aln-NC containing adenosine.

FIG. 13. Measurement of estradiol (E2) and microCT imaging in OVX mice. Estradiol levels in plasma.

FIGS. 14A-14G. Deficient CD73 and CD39 expressions and extracellular adenosine concentration in BM of OVX animals. Characterization of healthy (sham) and OVX animals 4 weeks after ovariectomy. (14A) Percentage and median fluorescence intensity of hematopoietic cells expressing CD73. (14B) Percentage and median fluorescence intensity of hematopoietic cells expressing CD39. (14C) Percentage and median fluorescence intensity of nonhematopoietic cells expressing CD73. (14D) Percentage and median fluorescence intensity of nonhematopoietic cells expressing CD39. (14E) CD73 gene expression and (14F) CD39 gene expression of cells from bone chips. (14G) Extracellular adenosine concentration in BM plasma of sham and OVX animals. n=5. *P<0.05, **P<0.01, ***P<0.001.

FIGS. 15A-C. Regulation of CD73 and CD39 cell membrane expressions and extracellular adenosine levels by ERs in osteoprogenitor cells. (15A) Quantification of CD73 and CD39 in osteoprogenitors in the absence or presence of E2 (100 nM) for 3 days. Single (ESR1 or ESR2) or dual (ESR1 and ESR2) ER knockdown (KD) by siRNA in primary mouse osteoprogenitors and analyzed after 3 days: (15B) Percentage of double-positive (CD73/CD39) cells in single knockdown and dual knockdown cells. (15C) In vitro adenosine levels normalized by cell number in single knockdown and dual knockdown cells. Control (scrambled) siRNA concentration for single knockdown and dual knockdown are 5 and 10 nM, respectively. n=5. *P<0.05, **P<0.01, ***P<0.001.

FIGS. 16A-16D. ER knockdown in osteoprogenitors and immunofluorescent staining of ectonucleotidase expression. Single (ESR1 or ESR2) or dual (ESR1 and ESR2) estrogen receptor knockdown (KD) by siRNA in primary mouse osteoprogenitors and analyzed after 3 days. (16A) Gene expression of ESR1 after treatment with estrogen receptor alpha (ESR1) siRNA. (16B) Gene expression of ESR2 after treatment with estrogen receptor beta (ESR2) siRNA. (16C) Gene expression of ESR1 and ESR2 after treatment with both estrogen receptor alpha (ESR1) and estrogen receptor beta (ESR2) siRNA. N=3. (16D) Quantification of percentage of cells co-expressing CD73 and CD39 immunofluorescence from fig. S2D. N=4. Single KD control siRNA concentration is 5 nM. Dual KD control siRNA concentration is 10 nM. *p<0.05, **p<0.01, ***p<0.001.

FIGS. 17A-17D. ER knockdown in osteoprogenitors and flow cytometric analyses of ectonucleotidase expression. Single (ESR1 or ESR2) or dual (ESR1 and ESR2) estrogen receptor knockdown (KD) by siRNA in primary mouse osteoprogenitors and analyzed after 3 days. (17A) Quantification of percent CD73-positive cells. (17B) Median fluorescence intensity of CD73-positive cells. (17C) Quantification of percent CD39-positive cells. (17D) Median fluorescence intensity of CD39-positive cells. N=5. Single KD control siRNA concentration is 5 nM. Dual KD control siRNA concentration is 10 nM. *p<0.05, **p<0.01, ***p<0.001.

FIGS. 18A-18C. Regulation of CD73 and CD39 cell membrane expression and extracellular adenosine levels by ERs in osteoclasts. (18A) quantification of CD73 and CD39 in primary mouse mononuclear cells undergoing osteoclast differentiation in the absence or presence of E2 (100 nM) for 3 days. Single (ESR1 or ESR2) or dual (ESR1 and ESR2) ER knockdown by siRNA during macrophage differentiation for 3 days and subsequent osteoclast differentiation for 6 days: (18B) Percentage of double-positive (CD73/CD39) cells in single knockdown and dual knockdown cells. (18C) In vitro adenosine levels normalized by cell number in single knockdown and dual knockdown cells. Control (scrambled) siRNA concentration for single knockdown and dual knockdown are 5 and 10 nM, respectively. n=4. *P<0.05, **P<0.01, ***P<0.001.

FIGS. 19A-19D. ER knockdown in osteoclasts and immunofluorescent staining of ectonucleotidase expression. Single (ESR1 or ESR2) or dual (ESR1 and ESR2) estrogen receptor knockdown by siRNA during macrophage differentiation for 3 days, and subsequent osteoclast differentiation for 3 days. (19A) Expression of ESR1 in primary mouse osteoclasts after treatment with estrogen receptor alpha (ESR1) siRNA. (19B) Expression of ESR2 in mouse osteoclasts after treatment with estrogen receptor beta (ESR2) siRNA. (19C) Expressions of ESR1 and ESR2 in mouse osteoclasts after dual treatment with estrogen receptor alpha (ESR1) and estrogen receptor beta (ESR2) siRNA. N=3. (19D) Quantification of percentage of cells co-expressing CD73 and CD39 immunofluorescence. N=4. Single KD control siRNA concentration is 5 nM. Dual KD control siRNA concentration is 10 nM. *p<0.05, **p<0.01, ***p<0.001.

FIGS. 20A-20D. ER knockdown in osteoclasts and flow cytometric analyses of ectonucleotidase expression. Single (ESR1 or ESR2) or dual (ESR1 and ESR2) estrogen receptor knockdown by siRNA during macrophage differentiation for 3 days, and subsequent osteoclast differentiation for 6 days. (20A) Quantification of percent CD73-positive cells. (20B) Median fluorescence intensity of CD73-positive cells. (20C) Quantification of percent CD39-positive cells. (20D) Median fluorescence intensity of CD39-positive cells. N=5. Single KD control siRNA concentration is 5 nM. Dual KD control siRNA concentration is 10 nM. *p<0.05, **p<0.01, ***p<0.001.

FIG. 21A-21D. ER knockdown of BM cells undergoing macrophage differentiation and flow cytometric analyses of ectonucleotidase expression. Single (ESR1 or ESR2) or dual (ESR1 and ESR2) estrogen receptor knockdown of mononuclear cells undergoing macrophage differentiation by siRNA for 3 days, and further macrophage differentiation for another 6 days. (21A) Quantification of percent CD73-positive cells. (21B) Median fluorescence intensity of CD73-positive cells. (21C) Quantification of percent CD39-positive cells. (21D) Median fluorescence intensity of CD39-positive cells. N=4. Single KD control siRNA concentration is 5 nM. Dual KD control siRNA concentration is 10 nM. *p<0.05, **p<0.01, ***p<0.001.

FIG. 22A-22E. Adenosine A2BR signaling promote osteogenic and inhibit osteoclast differentiation in vitro. (22A-22B) In vitro knockdown of adenosine A2BR using siRNA in primary mouse osteoprogenitor cells isolated from the BM for 2 days, followed by adenosine treatment (ADO; 30 μg/ml) for 7 or 14 days. Gene expression of (22A) osteoblast-specific marker and (22B) Opn. (22C-22E) In vitro knockdown of adenosine A2BR by siRNA in mouse mononuclear cells isolated from BM undergoing macrophage differentiation for 3 days, followed by osteoclast differentiation along with treatment of small-molecule adenosine (30 μg/ml) for 6 days. Gene expressions of (22C) osteoclast transcription factor Nfatc1, (22D) ACP5, and (22E) CTSK. *P<0.05, **P<0.01, ***P<0.001.

FIGS. 23A-23B. siRNA knockdown of A2BR and reverse transcriptase quantitative PCR. Gene expression of A2B receptor of (23A) osteoprogenitors and (23B) macrophages after treatment with adenosine A2B receptor siRNA for 3 days. CTL: scrambled siRNA (5 nM). A2BR: adenosine A2B receptor siRNA (5 nM). N=3. **p<0.01, ***p<0.001.

FIGS. 24A-24I. Adenosine A2BR agonist BAY 60-6583 attenuates bone loss in OVX animals. Administration of BAY 60-6583 and vehicle for 8 weeks in OVX animals (4 weeks after ovariectomy). Groups are compared to healthy control with no surgery and no treatment (CTL). (24A) Quantification of TRAP-positive cells on bone surface. n=4. (24B) Quantification of mineral apposition rate (MAR) from bone labeling images. (24C) Quantification of bone formation rate (BFR/BS) from bone labeling images. n=5. (24D-24I) Quantification of microCT images. (24D) BMD. (24E) BV/TV. (24F) Tb.N. (24G) Tb.Sp. n=5. Mechanical measurement for (24H) maximum load and (24I) stiffness of tibia. n=12. OV, ovariectomy, vehicle [dimethyl sulfoxide (DMSO)]; OB, ovariectomy, BAY 60-6583 (1 mg/kg). *P<0.05, **P<0.01, ***P<0.001.

FIG. 25. H&E staining and microCT of BAY 60-6583-treated mice. Quantification of femur bone volume (BV/TV). Sham: healthy, no treatment control. OV: ovariectomy surgery, vehicle (DMSO)-treated. OB: ovariectomy surgery, BAY 60-6583-treated. N=5. *p<0.05, **p<0.01.

FIGS. 26A-26B. Schematics of PBA-mediated adenosine sequestration. (26A) 3-(acrylamido)phenylboronic acid (PBA) contains boronic acid moiety (circled), which forms a dynamic covalent complex of cyclic boronate ester with cis-diol-bearing adenosine at physiological pH. (26B) PBA-based biomaterial patch sequesters extracellular adenosine at the fracture site while leveraging the adenosine surge after injury and sustains a localized adenosine signaling to accelerate tissue repair.

FIGS. 27A-27D. Adenosine molecules are sequestered by and released from PBA scaffolds in vitro. (27A) Representative UV/vis spectra show the absorption intensity of adenosine (in arbitrary units, a.u.), each corresponding to the amount of adenosine sequestered by a scaffold. Gray: adenosine sequestered by PBA₀; Cyan: adenosine sequestered by PBA_{0.5}; Blue: adenosine sequestered by PBA_{1.0}. (27B) Table lists the amount of adenosine sequestered by each scaffold and the corresponding sequestration efficiency and loading capacity (n=5 scaffolds for each group). (27C) Cumulative release of adenosine from the PBA_{1.0} scaffolds incubated in culture medium over 30 d (n=3 PBA_{1.0} scaffolds). (27D) Dot map comprises blue dots representing the adenosine released from the PBA_{1.0} scaffolds by Day 30 and gray dots representing the remaining adenosine in the scaffolds. In (27B) and (27C), data are presented as means (±s.d.).

FIGS. 28A-28D. PBA scaffolds sequester adenosine in vivo. (28A) Amount of adenosine sequestered in each subcutaneously implanted scaffold following the injection of saline, 0.25 mg/mL and 0.5 mg/mL adenosine solution, respectively (n=3 scaffolds for each injection condition). (28B) Table lists the injection conditions, the amount of adenosine injected, and the amount of adenosine sequestered in vivo by each scaffold (n=3 scaffolds for each injection condition). (28C) Extracellular adenosine level in bone marrow before and after the unilateral fracture. Bone marrow contents from both the fractured limb and the non-fractured limb were tested (n=3 bone marrow specimens for each condition). (28D) Scaffolds were excised from the fracture site at 3 d post implantation, and the sequestered adenosine was quantified (n=8 scaffolds for each group). All data are presented as means (±s.d.). One-way ANOVA with Tukey's multiple-comparisons test was used for statistical analysis in a; a two-tailed t-test (unpaired) was used for c and d. Significance is determined as *P<0.05, **P<0.01, ***P<0.001, and n.s. (not significant).

FIGS. 29A-29D. Adenosine-sequestered PBA scaffolds support osteogenic differentiation of hMSCs both in vitro and in vivo. (29A) PBA_{1.0} and PBA_{1.0}-ADO scaffolds loaded with hMSCs were cultured in vitro for 21 d. Expression levels of osteogenic markers (OCN, OPN, and OSX) were quantified as a function of time and presented as fold change against 18 s levels (n=3 purified gene specimens for each group). GM: growth medium; OM: osteogenic-inducing medium. (29B) Calcium content in each scaffold at 21 d (n=3 scaffolds for each group). (29C) Bone volume ratio (BV/TV) and bone mineral density (BMD) of the retrieved scaffolds were quantified based on microcomputed tomography (n=5 scaffolds for each group). (29D) Calcium content in each scaffold at Day 28 (n=5 scaffolds for each group). All data are presented as means (±s.d.). One-way ANOVA with Tukey's multiple-comparisons test was used for statistical analysis in (29B); a two-tailed t-test (unpaired) was used for (29A) (with reference to the group of PBA_{1.0} [GM] at 7 d), (29C) and (29D). Significance is determined as **P<0.01, ***P<0.001, ****P<0.0001, and n.s. (not significant).

FIGS. 30A-30B. PBA-containing biomaterial patches promote callus maturation during fracture healing. (30A) Bone volume ratio (BV/TV) of calluses at 14 d was quantified based on microcomputed tomography (n=6 mice for the cohort treated with PBA_{1.0}-ADO, n=7 mice for the cohort treated with PBA_{1.0} or PBA₀). (30B) Bone volume ratio (BV/TV) of calluses at 21 d was quantified (n=7 mice for the cohort treated with PBA_{1.0}-ADO, n=6 mice for the cohort treated with PBA_{1.0} or PBA₀). Data are presented as means (±s.d.). One-way ANOVA with Tukey's multiple-comparisons test was used for statistical analysis. Significance is determined as *P<0.05, **P<0.01, ***P<0.001, and n.s. (not significant).

FIGS. 31A-31C. PBA-containing biomaterial patches facilitate endochondral ossification and vascularization in fracture calluses. Quantification of the relative EMCN-positive vessel area to the callus area at 7 d (31A), 14 d (31B), and 21 d (31C) based on immunofluorescence images (n=5 mice from each treatment; each data point is averaged from 5 images for each mouse). Data are presented as means (±s.d.). One-way ANOVA with Tukey's multiple-comparisons test was used for statistical analysis. Significance is determined as *P<0.05, **P<0.01, and n.s. (not significant).

FIGS. 32A-32C. Synthetic scheme for the bone targeting nanocarrier with encapsulated adenosine. (32A) Synthesis of HA-MA-Aln; (32B) Synthesis of the ADO-ketal 2MAEA-ADO; (32C) Synthesis of the ADO containing bone targeting nanocarrier.

FIG. 33. ¹HNMR Spectrum of 2MAEA-ADO.

FIG. 34. Fold change of mineral deposition at day 21 in young mouse and old mouse bone marrow treated with growth medium (GM), GM+adenosine (ADO), and osteogenic inducing medium (OM).

FIG. 35. Adenosine sequestration by microgels containing PBA in young mice.

FIGS. 36A-36B. Bone mass density (36A) and bone volume (36B) in fracture healing of HA and HA-ADO treated aged mice.

### DETAILED DESCRIPTION OF THE INVENTION

The inventors have discovered that certain biomaterials can be harnessed to provide localized or locally-targeted delivery of therapeutic molecules to treat and/or prevent bone degeneration and/or to promote bone regeneration in diseases or disorders for which the promotion of bone regeneration and/or the prevention of bone degeneration is desired. The present disclosure is based, in part, on the discovery by the inventors that establishes the role of adenosine (ADO), an osteoanabolic molecule, functioning through P1 receptors (A1, A2A, A2B, and A3), in promoting bone formation as well as the role of the A2B receptor on osteogenic differentiation of stem cells. While promoting osteogenic differentiation, the same molecular pathways inhibit adipogenic differentiation. Adenosine promotes the bone forming function of osteoblasts and osteogenic differentiation of mesenchymal progenitor cells (thus promoting bone formation), and prevents over-activity of osteoclasts (thus preventing excessive bone degeneration). Accordingly, in one aspect, of the present invention provides a biomaterial for the systemic or localized delivery of osteoanabolic molecules to improve the targeting, retention and function of these molecules. The biomaterials disclosed herein may be used in the treatment and/or prevention of diseases or disorders for which the promotion of bone regeneration and/or the prevention of bone degeneration is desired.

### Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human in need of bone repair or bone formation.

Unless otherwise defined, all technical terms used herein have the same meaning as commonly understood by one of ordinary skill in the art to which this disclosure belongs.

### Biomaterials

In one aspect, the present invention provides a biomaterial for the targeted or localized delivery of a therapeutic agent, including a small molecule therapeutic. As used herein, “biomaterial” refers to any material suitable for in vivo applications. In certain instances herein, particular biomaterials of the disclosure may be referred to as nanocarriers or scaffolds. The biomaterials of the present invention comprise a polymer functionalized with, or conjugated to, a bioactive molecule binding moiety.

As used herein, “functionalized,” “functionalized with,” “conjugated,” and “conjugated to” are used interchangeably to refer to the chemical coupling, typically though covalent binding, of two or more molecules. Molecules may, for example, be copolymerized, or a moiety may be included as a substituent to a particular functional group or molecule. “Bioactive molecule” as used herein refers to a therapeutic agent for the treatment of diseases, disorders, and conditions, including those disclosed herein, and “bioactive molecule binding moiety” refers to a moiety able to reversibly bind to, or to dynamically covalently bind, a bioactive molecule.

The polymers used with the biomaterials disclosed herein may be any biologically compatible polymer. The polymers may be composed of a single type of monomer, or they may be copolymers of two or more types of monomers, and it is intended to be understood that any reference to a particular polymer herein is also intended to include a copolymer comprising the recited polymer. In certain embodiments, the polymer is a naturally occurring polymer including, but not limited to hyaluronic acid (HA). Other polymers that may be used with the invention include 2-(methacryloyloxy)ethyl acetoacetate (2MAEA) and polyethylene glycol (PEG).

The polymers may be modified or adapted as appropriate with chemical moieties to assist with the conjugation of moieties or molecules of interest, or with the polymerization or formulation of the biomaterials as disclosed herein. Such modifications are within the purview of one of skill in the art.

The bioactive molecule binding moiety allows for the biomaterials of the disclosure to reversibly bind to, and therefore deliver, bioactive molecules to a targeted or local site of interest. The ability to reversibly bind a bioactive molecule allows for the controlled or sustained release of the bioactive molecule at a site of interest. In certain embodiments, where the biomaterial may be used to treat and/or prevent bone degeneration and/or to promote bone regeneration, the bioactive molecule binding moiety may be an osteoanabolic molecule binding moiety, i.e. a moiety able to reversibly bind to, or to dynamically covalently bind, an osteoanabolic molecule.

In certain embodiments, the osteoanabolic molecule binding moiety is a boronate molecule, which can form dynamic covalent bonds with. e.g., cis-diol molecules such as adenosine. The boronate molecule may be, but is not limited to, phenylboronic acid (PBA). Representative biomaterials of the invention include a hyaluronic acid copolymer with PBA. In other embodiments, the osteoanabolic molecule binding moiety is a ketal group. Ketal groups are pH sensitive and can support the on-demand release of, e.g., adenosine.

Optionally, the biomaterials of the invention may further comprise, i.e. be chemically functionalized with, or complexed with, a bone targeting moiety. Inclusion of a bone targeting moiety allows for the targeted delivery of the biomaterial to bone by systemic administration (vs. local administration), thereby avoiding or diminishing off-target effects of the osteoanabolic molecule that might otherwise occur by way of systemic administration of the osteoanabolic molecule. The bone targeting moiety allow for the accumulation of the biomaterial in bone, including, e.g., the site of bone injury. The bone targeting moiety can be any apatite, hydroxyapatite, or bone binding agent such as an aptamer, peptide, small molecule, etc. In certain embodiments, the bone targeting moiety is a bisphosphonate molecule. Exemplary bisphosphonate molecules include, but are not limited to, etidronate, clodronate, tiludronate, pamidronate, neridronate, olpadronate, alendronate, ibandronate, risedronate, zoledronate. In certain embodiments, the bisphosphonate molecule is alendronate. The bone targeting moiety may be coupled to the polymer via amine coupling or any other suitable method.

The biomaterials may further comprise a bioactive molecule such as an osteoanabolic molecule. The term “osteoanabolic molecule” refers to any molecules that helps increase bone mass, including but not limited to, Vitamin D, adenosine, teriparatide, strontium ranelate, and the like. Such molecules can be “loaded” into the biomaterial (i.e. allowed to bind to the bioactive molecule/osteoanabolic molecule binding moiety) to enable the bioactive molecule to be administered for therapeutic use by way of the biomaterial. In certain embodiments the osteoanabolic molecule is an Adenosine A2B receptor (A2BR) agonist or is an adenosine compound. A2BR agonists include A2BR partial agonists. Examplary A2BR agonists that may be used with the invention include, but are not limited to, BAY 60-6583, NECA (N-ethylcarboxamidoadenosine), (S)-PHPNECA, LUF-5835, and LUF-5845. The adenosine compound may be adenosine, polyadenosine, or an analog or derivative of adenosine. The use of the biomaterial mitigates the short half-life and off-target effects of adenosine when administered without being complexed to the biomaterial.

Loading the biomaterial with adenosine (or another bioactive molecule of choice) allows for the introduction of exogenous adenosine to a site in need of bone regeneration and/or minimization of bone degeneration, either by way of systemic delivery (where a bone targeting moiety is utilized) or by local administration of the biomaterial (where a bone targeting moiety is optionally utilized). Alternatively, the biomaterial may be administered locally (e.g. as a patch at the site of bone injury) without being loaded with, e.g. adenosine. In such instances, the biomaterial may be “loaded” in vivo with endogenous adenosine, i.e. the biomaterial may be used to sequester adenosine at the site of bone injury or fracture. This use of the biomaterials of the invention leverages the innate adenosine surge after bone injury and sustains a localized adenosine signaling to accelerate tissue repair.

The biomaterial may be further functionalized with additional moieties and/or active agents which can be envisaged by one of skill in the art. The inclusion of any such additional moieties and/or agents would provide for the co-administration of these therapeutic agents with the bioactive molecules previously noted. The additional moieties and/or agents can also be included to assist in creating particular formats of the biomaterial. For example, as disclosed in Example 2, DBCO and azide groups can be used as dopants to form a stable porous scaffold.

Preparation of the biomaterials can be by any method known in the art or disclosed herein, for example by way of emulsion photopolymerization, the use of microfluidics, etc.

### Biomaterial Forms

The biomaterials disclosed herein may be formulated in different forms for use in different applications. Such forms include, but are not limited to, gels (hydrogels, nanogels, microgels), tablets, patches, transdermal patches or devices, pouches, devices, coatings for orthopedic implants, ointments, creams, and scaffolds (including macroporous scaffolds).

The biomaterials disclosed herein may be administered systemically (e.g. intraveneous, intraperitoneal) or locally (e.g. implantation, injection at site of defect), and may be degradable.

One of skill in the art will understand that particular forms may be best suited to particular applications, goals, and routes of administration. In non-limiting examples, nano-/microgels are suitable for systemic (intraveneous, intraperitoneal, etc.) administration, where the osteoanabolic molecule is delivered to bone tissue through targeting by the bone targeting moiety for the treatment of, e.g. osteoporosis. The nano-/microgels can also be used as building blocks to create injectable 3D scaffolds for local delivery. In this instance, the biomaterial could be functionalized with clickable units to allow for the formation of a porous space filling scaffold, which could be used for orthopedic injuries with space, such as tumor excised space, etc. In other examples, tablets are suitable for systemic (oral) administration, patches for local administration (e.g. at the site of a bone fracture), and creams or ointments, as well as transdermal patches or devices, for transdermal delivery.

In some embodiments, a the biomaterials can be formulated as a pouch or device to be used as a surgically-implanted replenishable device, where the level of the osteoanabolic agent in the pouch or device can be re-loaded as needed noninvasively through local injection (e.g. injection of adenosine into the biomaterial pouch). Alternatively, the pouch or device can sequester endogenous adenosine.

In certain instances, the nano-/microgels may be spherical in shape with a diameter in the range of 0.01 to 500 μm. In setting forth this range, it is intended that any range within the stated range, or any specific value falling within the range, be included even if not specifically enumerated. Accordingly, the nano-/microgels may have a diameter of 0.1-400 μm, of 1-200 μm, of 10-200 μm, of 60-100 nm, of 90-110 nm, of about 0.1 _{[}tm, of about 1 μm, of about 100 μm, of about 200 μm, etc.

### Pharmaceutical Compositions and Administration

The biomaterials provided herein can be administered to a subject, either alone or in combination with a pharmaceutically acceptable excipient and/or carrier, in an amount sufficient to induce an appropriate biological response (e.g., increasing bone mass).

An effective amount of the biomaterial compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the vaccine. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23,24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The present disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A “pharmaceutically acceptable excipient” or “diagnostically acceptable excipient” includes but is not limited to, sterile distilled water, saline, phosphate buffered solutions, amino acid-based buffers, or bicarbonate buffered solutions. An excipient selected and the amount of excipient used will depend upon the mode of administration. Administration may in certain instances comprise an injection, infusion, or a combination thereof.

An effective amount for a particular subject/patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A dosing schedule of, for example, once/week, twice/week, three times/week, four times/week, five times/week, six times/week, seven times/week, once every two weeks, once every three weeks, once every four weeks, once every five weeks, and the like, is available for the present disclosure. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are possible cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The biomaterials according to the present disclosure may also be administered with one or more additional therapeutic agents (e.g., other osteoanabolic molecules, bone growth/bone healing promoting compounds, etc.). The biomaterials may be functionalized with the one or more additional therapeutic agents, or the one or more additional therapeutic agents may be co-administered with the biomaterials. Methods for co-administration with an additional therapeutic agent are well known in the art (Hardman, et al. (eds.) (2001) Goodman and Gilman's The Pharmacological Basis of Therapeutics, 10th ed., McGrawHill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

Co-administration need not refer to administration at the same time in an individual, but rather may include administrations that are spaced by hours or even days, weeks, or longer, as long as the administration of multiple therapeutic agents is the result of a single treatment plan. The co-administration may comprise administering the biomaterial according to the present disclosure before, after, or at the same time as the one or more additional therapeutic agents. In one exemplary treatment schedule, the biomaterial of the present disclosure may be given as an initial dose in a multi-day protocol, with one or more additional therapeutic agent given on later administration days; or the one or more additional therapeutic agents given as an initial dose in a multi-day protocol, with the biomaterial of the present disclosure given on later administration days. On another hand, one or more additional therapeutic agents and the biomaterial of the present disclosure may be administered on alternate days in a multi-day protocol. In still another example, a mixture of one or more additional therapeutic agents and the biomaterial of the present disclosure may be concurrently. This is not meant to be a limiting list of possible administration protocols.

An effective amount of a therapeutic agent is one that will increase bone mass/bone healing by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9% as compared to no treatment.

Specific dosing regimens are within the purview of one of ordinary skill in the art. An exemplary dose of the biomaterials of the invention is 30 mg/kg of adenosine (with the biomaterial).

Formulations of therapeutic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGrawHill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Phalmaceutical Dosage Farms: Disperse Systems, Marcel Dekker, NY; Weiner and Kotkoskie (2000) Excipient Toxicity and Safety, Marcel Dekker, Inc., New York, N.Y.).

The biomaterials according to the present disclosure may be administered to a subject in a number of ways, including but not limited to, oral, aerosol, intranasal, injection, systemic, parenteral, subcutaneous, intravenous, intramuscular, intrathecal, interperitoneal and rectal. In some embodiments, the biomaterials are administered systemically.

### Methods

The biomaterials of the present disclosure may be used to treat and/or prevent in a subject any disease, disorder, or condition where for which the subject would benefit from reduced bone degeneration and/or the promotion of bone regeneration. Thus, in accordance with the disclosure, the present invention includes, but is not limited to, methods of promoting osteoblastogenesis and/or decreasing osteoclastogenesis, methods of treating and/or preventing a low bone mass condition (e.g. osteoporosis, osteopenia), methods of treating bone fracture, methods of promoting bone fracture healing, methods of promoting bone regeneration, methods of treating a bone disease comprising bone degeneration, methods of treating bone degeneration, methods of activating A2BR to promote bone repair, methods of repairing skeletal defects, methods of enhancing bone mineral density, methods of enhancing bone volume, methods of enhancing trabecular bone parameters (e.g. connectivity density and trabecular spacing), methods of enhancing the outcome of orthopedic implant surgery, methods of enhancing the innate ability of bone repair tissue to repair bone, methods of sequestering endogenous adenosine at the site of bone injury, and methods of aiding in age-related bone degeneration in a subject in need thereof, comprising administering to the subject a biomaterial as disclosed herein.

As used herein, a “low bone mass condition” refers to any condition characterized by the inability of a subject to regenerate new bone as quickly as it absorbs old bone.

The biomaterials of the invention have been described above. In accordance with, e.g., the bone targeting moiety of the biomaterials, the biomaterials can be administered systemically and will localize to the bone tissue to effect repair. Alternatively, forms such as a patch, a scaffold, etc. can be injected or implanted into the area requiring repair. Here, the biomaterial can sequester the surge of endogenous adenosine at the site of bone injury to allow for a sustained release of adenosine over time. Or alternatively, the biomaterial can be loaded with adenosine prior to injection or implant into the area requiring repair. Adenosine can then, if desired, be replenished by injection of adenosine at the area of the implant. In some instances, orthopedic implants can be coated with the biomaterials of the invention prior to implantation to enhance the outcome of the implant surgery. In each of these cases, the osteoanabolic molecule will be localized to the bone tissue and/or the site of injury in order to promote bone regeneration and/or decrease bone degeneration, and the osteoanabolic molecule can be released in a controlled/sustained fashion while minimizing off-target effects.

### EXAMPLES

### Example 1

### Bone Targeting Delivery System for Systemic Administration of Therapeutics

### Introduction

Extracellular adenosine has been shown to play a key role in maintaining bone health and could potentially be used to treat bone loss. However, systemic administration of exogenous adenosine to treat bone disorders is challenging given the ubiquitous presence of adenosine receptors in different organs and the short half-life of adenosine in circulation. Towards this, a bone-targeting nanocarrier was developed and its potential for systemic administration of adenosine was determined. The nanocarrier (NC), synthesized via emulsion photopolymerization, is comprised of hyaluronic acid (HA) copolymerized with phenylboronic acid (PBA), a moiety that can form reversible bonds with the cis-diol groups of adenosine molecules. The bone binding affinity of the nanocarriers was achieved via alendronate (Aln) conjugation. Nanocarriers functionalized with the alendronate (Aln-NC) showed a ˜45% higher accumulation in the mice vertebrae in vivo compared to the NCs lacking alendronate molecules. Systemic administration of adenosine via bone-targeting NCs (Aln-NC) showed attenuated bone loss in ovariectomized (OVX) mice. Furthermore, bone tissue of mice treated with adenosine loaded Aln-NC displayed comparable trabecular bone characteristics to healthy controls as shown by microcomputed tomography, histochemical analyses, bone labeling, and mechanical strength. Overall, the results demonstrate the use of a bone-targeting nanocarrier towards systemic administration of adenosine and its application in treating bone degenerative diseases such as osteoporosis.

### Materials and Methods

### Materials

Hyaluronic acid (molecular weight 40 kDa, HA40K-5) was purchased from Lifecore, USA. Methacrylic anhydride (276685), N-hydroxysuccinimide (NHS, 130672), sodium hydroxide (795429), adenosine (A4036) and mineral oil (M5904) were obtained from Millipore Sigma, USA. 1-Ethyl-3-(3-dimethylaminopropyl) carbodiimide hydrochloride (EDC, D1601) and 3-acrylamido phenylboronic acid (3-APBA) (A3199) were obtained from TCI Chemicals, USA. Alendronate (J61397) was purchased from Alfa Aesar, USA. Cyanine 7.0 amine (55000) was purchased from Lumiprobe. Dialysis bags (Molecular weight cut off, MWCO was 2.0 and 3.5 kDa) were obtained from Spectrum, USA. ABIL EM90 surfactant (420095-L-151) was obtained Universal Preserv-A Chem INC, Germany. Hexane, acetone, ethanol, and dimethyl sulfoxide (DMSO) were purchased from Millipore-Sigma, USA; the solvents were of ACS or spectroscopic grade. Genesys 10S UV-VIS spectrometer was used to record the UV-visible spectra. FTIR spectra were recorded on Thermo Electron Nicolet 8700 FTIR spectrometer. NMR spectra were recorded in FFSC 400 MHz Agilent/Varian Inova spectrometer. FEI Tecnai G2 20 TWIN electron microscope was used to generate the TEM images.

### Synthesis of Hyaluronic Acid Methacrylate (HA-MA)

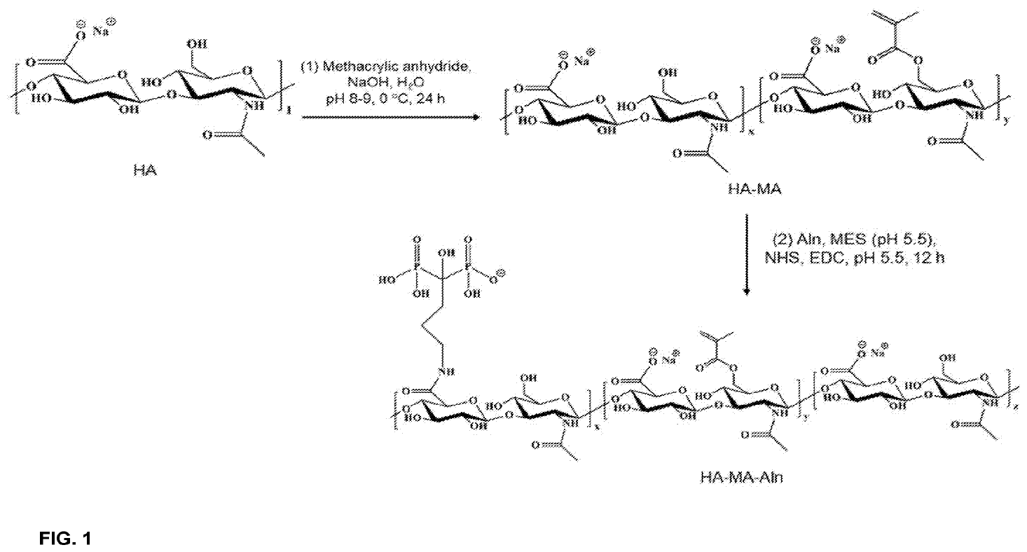

Photopolymerizable methacrylate group was introduced into HA via esterification of the hydroxyl group upon reacting HA with methacrylic anhydride (FIG. 1). Briefly, HA (600 mg) was dissolved in deionized (DI) water. Methacrylic anhydride (4.4 mL) was added to the HA solution and the pH of the reaction mixture was adjusted to 8-8.5 by adding 5 N NaOH. The reaction was continued for about 24 h at 4° C. Excess of ice-cold ethanol-acetone mixture (1:1) was added to precipitate the product. The precipitate was filtered, washed several times with ice-cold ethanol-acetone mixture. Next, the polymer was dissolved in DI water and dialyzed for 4 days (using 3.5 kDa membrane) against DI water. The solution was freeze dried to obtain the methacrylated HA. The polymer was characterized by using a combination of FTIR and ¹HNMR spectroscopy. FTIR spectra of the modified HA showed the presence of peaks corresponding to ester C═O and methacrylate C═C stretching frequencies at 1720 cm⁻¹ and 1610 cm⁻¹ respectively, confirming successful methacrylation. The degree of methacrylation, determined via ¹HNMR spectroscopy, was found to be 30±2% per dimeric repeating unit.

### Synthesis of Alendronate-Conjugated HA-MA (HA-MA-Aln)

HA-MA was modified with the bone targeting agent alendronate (Aln) via amide coupling reaction between the carboxylic acid group of HA-MA and the amine group of Aln (FIG. 1). Briefly, HA-MA (400 mg) was dissolved in IVIES buffer of pH 5.5 to yield a concentration of 10 mg/mL. EDC (175 mg) and NHS (105 mg) were gradually added to HA-MA solution at 15 min intervals. After 30 min, Aln (74.2 mg) was added to the reaction mixture. The reaction was continued for about 12 h at room temperature. The mixture was then dialyzed by using a 3.5 kDa membrane against DI water for 4 days and the resulting purified solution was lyophilized to obtain alendronate conjugated HA-MA (HA-MA-Aln). The polymer was characterized by using FTIR and ¹HNMR spectroscopy. The degree of Aln conjugation, determined via ¹HNMIR spectroscopy, was found to be ˜18±1% with respect to the dimeric repeating unit of HA.

### Synthesis of Cy7 Dye Conjugated HA-MA or HA-MA-AM (HA-MA-Cy7 or HA-MA-Aln-Cy7)

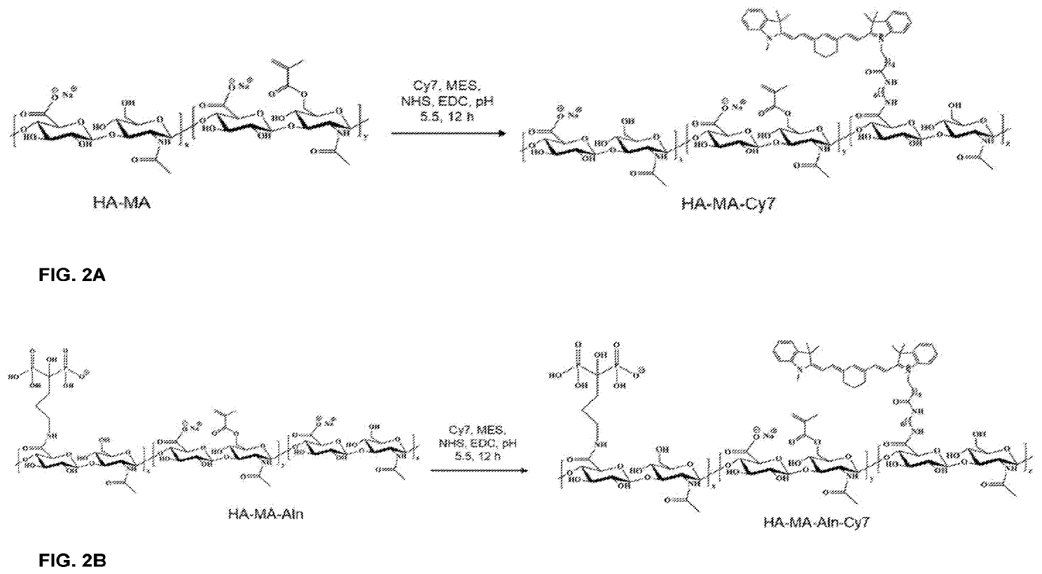

To prepare the fluorescently labelled polymers, HA-MA or HA-MA-Aln were conjugated with a fluorescent dye, Cy7, via amide coupling reaction between the free carboxylic acid groups of HA-MA or HA-MA-Aln and the amine group of Cy7 (FIGS. 2A and 2B). Briefly, HA-MA or HA-MA-Aln (100 mg) was dissolved in a mixture of 1:1 DI water:DMSO to create a concentration of 5 mg/mL. EDC (69 mg) and NHS (41.4 mg) was added to the polymer solution at 15 min intervals. After 30 min, cyanine 7 amine (Cy7, 5-6 mg), dissolved in 5 mL DMSO was added to the reaction mixture, and the reaction was continued for about 48 h at room temperature. The mixture was dialyzed (3.5 kDa membrane) against 1:0.1 mixture of DI water:DMSO for 1 day followed by DI water for 4-5 days. The solution was lyophilized to obtain the dye conjugated polymer. The polymer was characterized via a combination a UV-visible, FTIR and ¹HNMR spectroscopy. The successful conjugation of the dye to the polymer backbone was confirmed by UV-visible spectroscopy as the spectra showed typical Cy7 absorption at ˜750 nm. The dye content was determined via ¹HNMR spectroscopy and was found to be ˜3-4% (with respect to the dimeric repeating unit of HA) as indicated by the presence of aromatic protons (at 7.1-7.3 ppm) from Cy7.

### Nanocarrier Synthesis and Purification

The nanocarrier was prepared via inverse emulsion photopolymerization. Briefly, HA-MA (50 mg), HA-MA-Aln (55 mg), HA-MA-Cy7 (52.5 mg), or HA-MA-Aln-Cy7 (57.5 mg) were dissolved in DI water (550 μL). 3-Acrylamido phenylboronic acid (3-APBA) (92.5 mg) was dissolved in ethanol (400 μL). Both the solutions were then mixed together. Subsequently, 50 μL of the photoinitiator LAP (2% w/v in DI water) was added to the polymer-PBA mixture. The final solution was emulsified in a continuous phase consisting of mineral oil (10 mL) containing 10% w/v ABIL EM 90 surfactant through ultrasonication (probe sonicator, 15-18 kW output) for 90 sec at 4° C. The nanodroplets were crosslinked via UV irradiation for 10 min under constant stirring at 300 rpm. To remove the continuous phase, the emulsion was diluted (1:10) with a chilled mixture of 1:1 acetone:hexane. The nanocarriers were pelleted down by centrifugation (15000 rpm, 15 min) and the supernatant was discarded. The pellet was washed three times with the 1:1 acetone:hexane mixture. Next, the nanocarriers were dispersed in 10 mL 1:1 water:ethanol mixture, dialyzed against water, freeze dried and stored at −20° C. until use. The fluorophore tagged nanocarriers were prepared by using the Cy7 conjugated polymers (HA-MA-Cy7 or HA-MA-Aln-Cy7) following the same protocol. The nanocarriers contained approximately 1.8-2.2 nmol of fluorophore per milligram of the carrier as determined by the UV-visible absorption spectroscopy. A standard calibration curve for Cy7 at ˜750 nm vs concentration (1-31.25 μg/mL) was prepared and used to determine the PBA content in the polymers.

### Characterization of the Nanocarriers

The nanocarriers were characterized via a combination of UV-Visible, FTIR, ¹HNMR spectroscopy and transmission electron microscopy (TEM). A fixed amount of the freeze-dried nanocarrier was suspended in 1:1 water-ethanol mixture. Absorbance was measured by using a UV-visible spectrophotometer (200-800 nm). A standard calibration curve for 3-APBA at ˜255 nm vs concentration (7.8-125 μg/mL) was made and used to determine the PBA content in the nanocarriers. Freeze-dried nanocarriers (5-10 mg) were suspended in 600 μL D₂O followed by the addition of 20 μL of 5N NaOH in D₂O, and ¹HNMR spectra were recorded by using a 400 MHz Varian spectrometer. To image the nanocarriers by transmission electron microscopy (TEM), 1-2 mg of the freeze-dried nanocarriers was suspended into 1:15 water-ethanol mixture. Nanocarrier suspension (2 μL) was then cast onto the 300 mesh holy carbon grid, dried overnight at 50-60° C., and imaged by using the Tecnai 200 kV electron microscope at an operating voltage of 80 kV. The particle size was estimated by using Image J software. A minimum of three images taken at three different places of the TEM grid were analyzed. The Cy7 content in the nanocarriers was determined via UV-Visible spectroscopy using the standard calibration curve for Cy7 at ˜750 nm vs concentration (1-31.25 μg/mL).

### Adenosine Encapsulation and Release

The freeze-dried nanocarriers were soaked overnight in 7 mg/mL adenosine solution in PBS at pH 8.5. The nanocarriers were then concentrated either via centrifugation at 21000 rpm for 20 min or by using amicon centrifugation filter with MWCO of 100 kDa, washed with PBS, and freeze dried. The adenosine-loaded nanocarriers were suspended in 10% FBS containing alpha-MEM media to yield a concentration of 5 mg/mL. Approximately 1 mL of the suspension was transferred into a dialysis bag with a MWCO of 2 kDa. The bag was placed in a 15 mL falcon tube containing 9 mL media and incubated at 37° C. At predetermined time intervals, 2 mL of the media was removed and supplemented with 2 mL fresh media. The adenosine content in the media was measured by using UV-visible spectrophotometer at 260 nm wavelength. A standard calibration curve of adenosine (15.6-125 μg/mL) was prepared in alpha-MEM medium and was used to estimate the adenosine content.

### In Vitro Bone Binding Affinity

The ability of the nanocarriers to bind to bone tissue was assessed in vitro by using bone (femur) chips collected from 8-12 weeks old female C57BL/6J mice. The bone marrow from the femur was flushed out and the bone was cut into small pieces of ˜3-4 mm. A fixed amount of the Cy7 conjugated nanocarriers was suspended in α-MEM media containing 10% FBS to yield a concentration of 1 mg/mL. The bone chips were incubated with the nanocarrier at 37° C. under constant shaking at 150 rpm for about 2 hrs. The bone chips were removed and washed with PBS to remove the unbound nanocarriers. The fluorescence intensity was recorded using an in vivo imaging system (IVIS Kinetics) with a 750 nm excitation wavelength and 780 nm emission wavelength. The normalized radiant efficiency was divided by the surface area of the bone chip and the results were expressed as radiant efficiency/mm².

### In Vivo Biodistribution

All animal studies were performed with the approval of Institutional Animal Care and Use Committee (IACUC) at Duke University and in accordance with the guidelines of the National Institutes of Health (NIH). Athymic nude mice (NU(NCr)-Foxn1^{nu}, 8 weeks old) from Charles River Laboratory were used for the experiment. The mice were divided into two groups (n≥5 for each group). The dye containing nanocarriers with or without bone targeting alendronate was suspended in saline. 100 μL of the suspension, which approximates to a dye concentration of ˜2.1 nM, was injected intravenously via the tail vein with a single dose. At designated times after i.v. injection, mice were anesthetized using isoflurane inhalation and whole-body images were acquired using an IVIS imaging system. Some of the animals were euthanized at 72 hrs post injection and major organs/tissues such as vertebra, femur, tibia, heart, lungs, liver, spleen, kidneys, brain, and muscle were harvested. The wet weight of organs was recorded and imaged using IVIS. Fluorescence intensity after background-subtraction was normalized to organ weight and the amount of fluorophore conjugated nanocarriers present in each organ was estimated from the fluorescence intensity. Data analysis was carried out by using Living Image software and the results were expressed as radiant efficiency/g of the organs.

To examine the distribution of the nanocarriers within the bone tissue, lumbar vertebrae (L4 segment) and the femur (proximal) were excised, cleaned of excess soft tissues, and placed in 4% paraformaldehyde overnight at 4° C. Following overnight fixation, undecalcified tissues were incubated in 30% sucrose, then embedded in cryomatrix (ThermoFisher) and cryosections (10 μm) were prepared using CryoJane tape transfer system using a Leica cryotome. Sections were stained with Hoechst 33342 for nuclei. Fluorescence images were then taken with a Zeiss Axio Observer Z1 microscope. Representative images of sections showing both cortical and trabecular regions in the vertebral column and proximal femur for both nanocarriers with and without Aln were taken. To detect Cy7, the sections were imaged using a 710/75 band pass excitation filter and 810/90 nm band pass emission filter and are shown in pseudo red color. Hoechst 33342 were imaged at 365 nm excitation wavelength and 445/50 nm band pass emission filter.

### In Vivo Administration of Nanocarrier

Ovariectomized female C57BL/6J mice (12 weeks old; Jackson Laboratory, Bar Harbor, Me.) were used. Animal grouping and treatment included: mice with no OVX surgery, i.e., healthy mice (control, CTL), mice with OVX surgery (O), OVX mice treated with Aln-NC without adenosine (OH), OVX mice treated with adenosine containing Aln-NC (OHA) by tail vein injection. Administration of nanocarriers started 4 weeks after OVX surgery and the treatment was continued twice a week for 8 weeks. Mice were treated ˜90 mg/kg body weight of Aln-NC and ˜120 mg/kg body weight adenosine containing Aln-NC. The adenosine dosage was ˜30 mg/kg body weight of mice.

### Bone Labeling

Animals were administered with calcein (Sigma-Aldrich) at a concentration of 10 mg/kg body weight at 14 days prior to sacrifice and alizarin-3-methyliminodiacetic acid (30 mg/kg body weight; Sigma-Aldrich) 9 days post-administration of calcein. Collected vertebrae were fixed in 4% PFA at 4° C. for 1 d and stored in 70% ethanol. The undecalcified samples were incubated in 30% sucrose for 24 hrs, embedded in cryomatrix, cryo-sectioned with CryoJane Tape transfer, and imaged for bone labeling. Bone formation rate (BFR) and mineral apposition rate (MAR) were calculated from parameters measured from images using ImageJ software. BFR=MAR×(MS/BS). MAR=(irL.Wi)/time interval. Distance between the double fluorescent labels (interlabel width, irL.Wi), divided by the time interval. Mineralizing surface (MS/BS)=100*(dL.Pm+(0.5×sL.Pm))/B.Pm. Perimeter of double labeled bone (dL.Pm) plus perimeter of one half of the singly labeled bone (0.5×sL.Pm) as a fraction (%) of the total bone perimeter (B.Pm).

### Microcomputed Tomography

Vertebrae (L3-L5) and femur were collected, fixed in 4% paraformaldehyde (PFA) at 4° C. for 1 d, and rinsed thoroughly with PBS. The fixed samples were placed in 50 mL centrifuge tubes with styrofoam spacers and loaded into a p.-CT scanner (vivaCT 80, Scanco Medical, Wayne, Pa.). The samples were scanned at 55 keV at a pixel resolution of 10.4 μm. The reconstruction of the images was performed using μ-CT Evaluation Program V6.6 (Scanco Medical), followed by generation of radiographs and 3D models using μ-CT Ray V4.0 (Scanco Medical). Bone mineral density of the tissue was quantified and presented as a percentage of bone volume (BV) per total volume (TV) (% BV/TV) using the phantom as a reference based on 100 contiguous slices. Trabecular number (Tb.N), trabecular spacing (Tb. Sp), connectivity density (Conn. D), trabecular thickness (Tb. Th) were quantified by CTAn software.

### Mechanical Measurement

Tibiae were used to measure the mechanical properties. After removing the soft tissues, tibia samples were wrapped in wet tissue and frozen at −20° C. Sixteen hrs prior to the measurement, the samples were placed at 4° C., and then in room temperature an hour before the measurement. Four-point bending mode of Electroforce 3220 (TA Instruments, New Castle, Del.) instrument with 225 N load cell was used for the test. Samples were aligned on the fixtures and the load was applied perpendicular to the principal axis of the tibia. The span length of the bottom support was 9.2 mm while the top span length was 2.8 mm. Bending test was performed in displacement control mode at a loading rate of 0.025 mm/sec. Load-displacement data was recorded at a data acquisition rate of 10 Hz. Displacement was tared at the first data point at which the load equaled or exceeded 1N. Maximum load is the highest load (N) before the sample fractures. Bending stiffness (N/mm) was calculated as the slope of load vs. displacement between 30-70% of maximum load to failure in the linear region and work-to-fracture (N-mm) was determined as area under the curve.

### Histological Staining

Vertebral samples were fixed with 4% paraformaldehyde (PFA) at 4° C. for 1 d and decalcified using 10% ethylenediaminetetracetic acid (EDTA, pH 7.3) for 2 weeks at 4° C. The samples were gradually dehydrated using increasing concentrations of ethanol and incubated in Citrisolv (Decon Laboratories) until equilibrium was reached. Following dehydration, samples were immersed in a mixture of 50% (v/v) Citrisolv and 50% (w/w) paraffin (General Data Healthcare) for 30 min. at 70° C. The samples were embedded in paraffin and 7 μm thick sections were generated by using a rotary microtome (Leica, RM2255). Before staining, the sections were deparaffinized using CitriSolv and subsequently rehydrated with decreasing concentration of ethanol until the samples were equilibrated with DI water. Hematoxylin and eosin (H&E) staining was performed by first incubating the samples in hematoxylin solution (Ricca Chemical) for 3 min followed by incubation in Eosin-Y solution (Richard-Allan Scientific) for 2 s. Stained sections were gradually dehydrated using increasing concentrations of ethanol until equilibrium was reached. Tartrate-resistant acid phosphatase (TRAP) staining was performed by incubating rehydrated sections in an acetate buffer (0.2 M) containing sodium L-tartrate dibasic dihydrate (50 mM) at pH 5 for 20 min at room temp followed by incubating with naphthol AS-MX phosphate disodium salt (Sigma, N5000-1G; 0.5 mg/mL) and Fast Red TR Salt 1,5-naphthalenedisulfonate (Sigma, F6760-5G; 1.1 mg/mL) dissolved in the same buffer for 1.5 h at 37° C. Sections were mounted using permount mounting medium (ThermoFisher) and imaged using a Keyence BZ-X700 microscope.

### Statistical Analyses

All numerical values are expressed as means±standard deviation. Data was subjected to either one-way analysis of variance (ANOVA) or two-tailed Student's t-test with post hoc Tukey-Kramer test for multiple comparisons. P-Value of less than 0.05 was considered statistically significant and indicated with an asterisk. All statistical analyses were performed with GraphPad Prism 8.1.1.

### Results

### Synthesis and Characterization of Nanocarriers

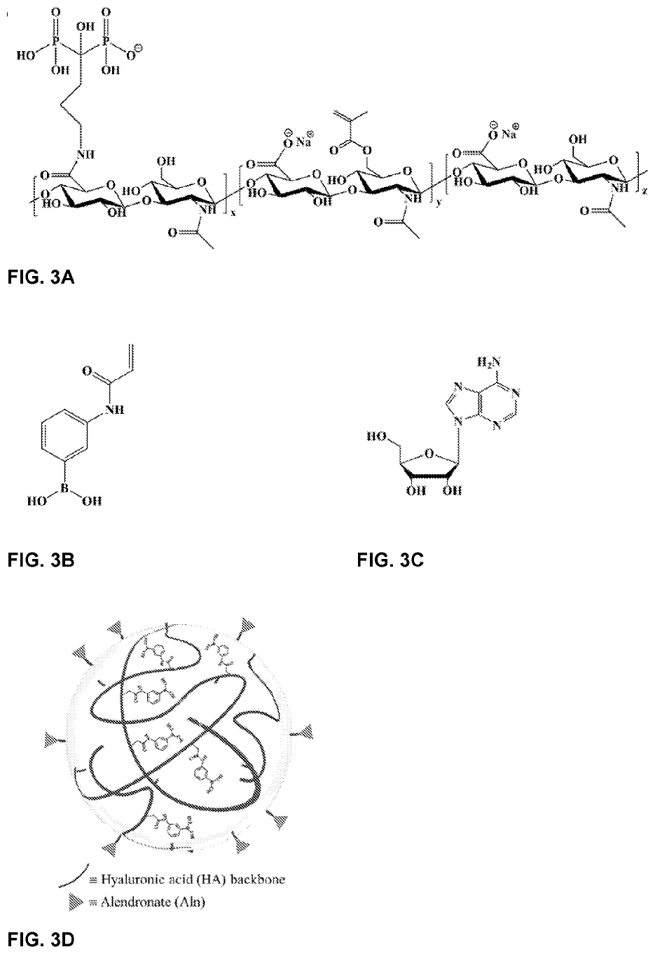

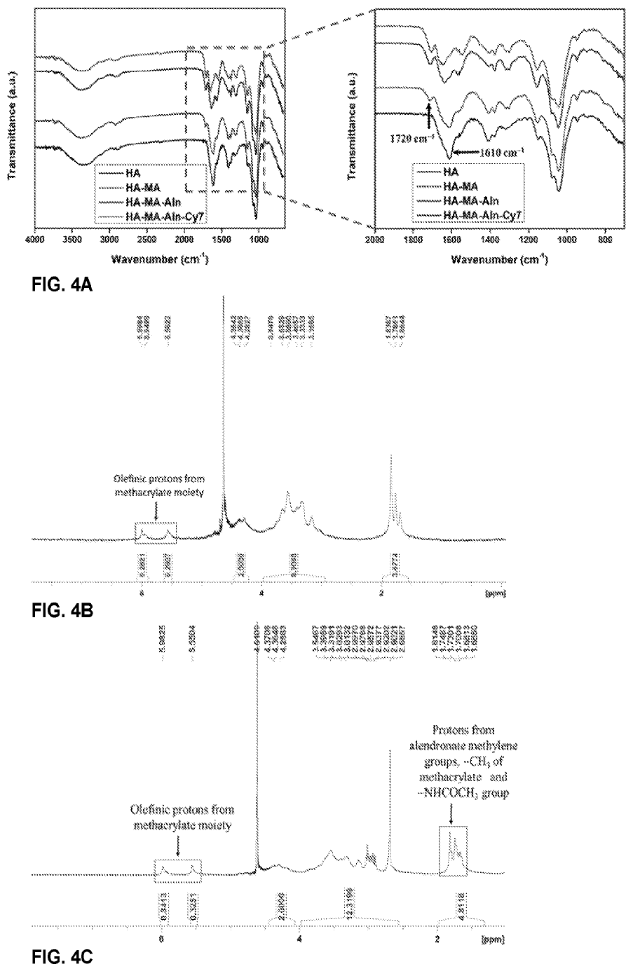

HA was chemically modified to introduce polymerizable methacrylate (MA) and bone targeting Aln groups. The modified-HAs (HA-MA or HA-MA-Aln with 30±2% degrees of methacrylation and 18±1% degrees of Aln conjugation with respect to the dimeric repeating unit of HA) were copolymerized with 3-(acrylamido)phenylboronic acid (3-APBA) in emulsion suspension polymerization (FIGS. 3A-3D). (Burdick 2005; Raemdonck 2009). The details about the synthesis and characterization of the modified-HAs (HA-MA and HA-MA-Aln) are provided above and in the figures (FIG. 1 and FIGS. 4A-4C).

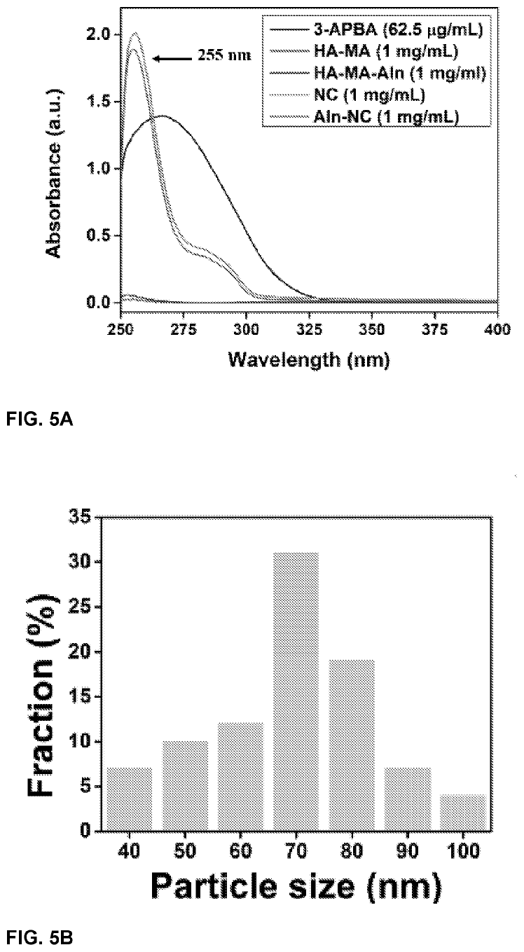

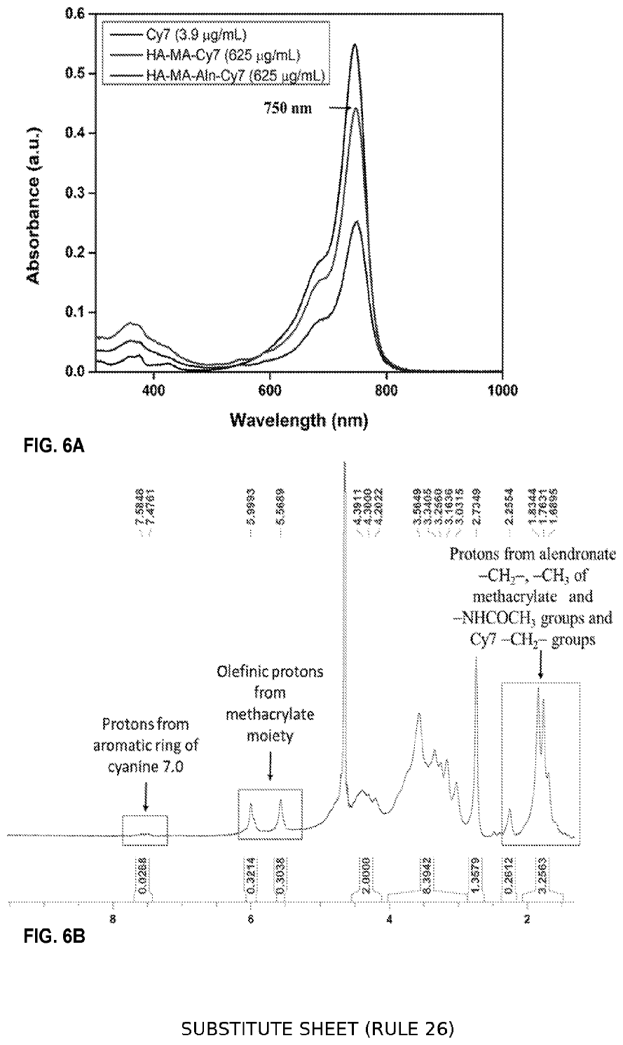

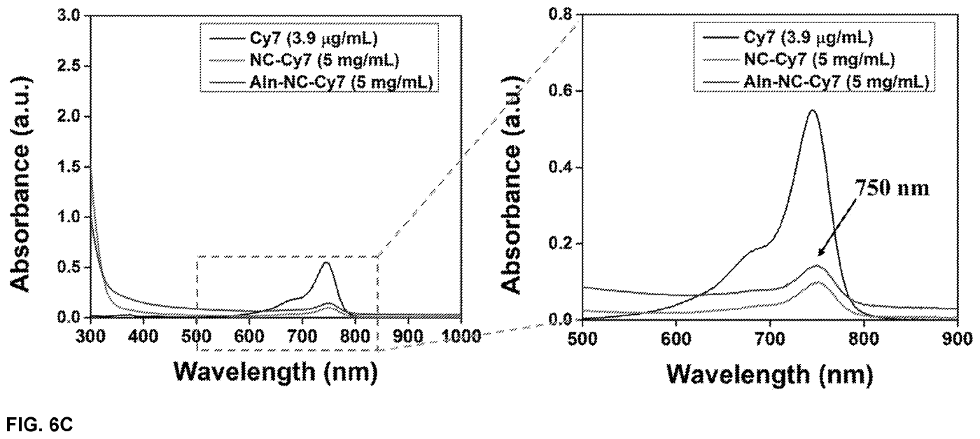

Two types of nanocarriers (nanocarriers with and without Aln; hereafter named as NC and Aln-NC, respectively) were synthesized for their ability to load adenosine and target bone tissue. The NCs were characterized via a combination of Fourier-transform infrared (FTIR) spectroscopy, proton nuclear magnetic resonance (¹HNMR) spectroscopy, ultraviolet-visible (UV-vis) spectroscopy and transmission electron microscopy (TEM). FTIR spectra of the NCs showed absorptions at 1558-1610 cm⁻¹ and 1352 cm⁻¹ which are characteristics of C═C stretching frequencies of the benzene ring and O—B—O bending of PBA, respectively, indicating successful incorporation of PBA moieties into the NCs. The ¹HNMR spectra of the nanocarriers exhibited diminished methacrylate peaks (at 5.5-6.1 ppm) and showed appearance of new peaks corresponding to aromatic protons (at 6.8-7.1 ppm), further confirming the conjugation of PBA to the modified-HA. In addition, peaks at 1.6-1.9 ppm corresponding to methylene protons in the Aln-NC confirmed the presence of Aln groups in the nanocarrier. The extent of PBA incorporation in the nanocarriers, determined from the UV-vis spectra, was found to be ˜92% (with respect to the amount of 3-APBA used for copolymerization with modified HA) (FIG. 5A). TEM images of the nanocarriers showed spherical particles with diameter between 60 and 100 nm (FIG. 5B). Fluorescent dye cyanine 7 (Cy7) conjugated nanocarriers were synthesized and characterized similarly. Details are provided above and in FIGS. 2A-2B and FIGS. 6A-6C.

### Adenosine Loading

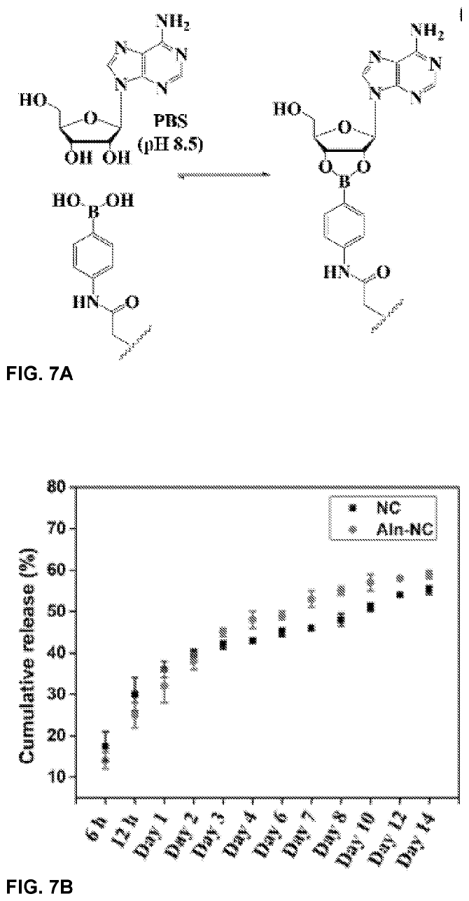

The adenosine molecules were loaded by incubating the nanocarriers in excess adenosine solution in PBS (pH 8.5) for about 12 h (FIG. 7A). The nanocarriers had a loading efficiency (the amount of PBA moieties involved in adenosine binding) of ˜56% with a loading capacity (weight percentage of adenosine in the nanocarrier) of ˜31%. Time dependent analyses (2-24 hrs) showed increasing incubation time beyond 12 hrs did not have a significant effect in adenosine loading (Table 1). The release profile of loaded adenosine from the nanocarriers was examined in alpha-MEM medium containing 10% fetal bovine serum. Approximately 45-50% of the encapsulated adenosine was found to be released within the initial 4 days, and the adenosine release almost plateaued thereafter (FIG. 7B). The presence of the Aln functional group had no effect on the adenosine loading or release (FIG. 7B).

TABLE 1
Time dependent adenosine loading into nanocarriers

Adenosine loading capacity (%)

| Nanocarriers | 2 h | 6 h | 12 h | 24 h |
|---|---|---|---|---|
| NC | 20 ± 1 | 27 ± 1 | 32 ± 2 | 28 ± 2 |
| Aln-NC | 18 ± 2 | 25 ± 1 | 31 ± 2 | 29 ± 2 |

### Bone Targeting Efficacy and Biodistribution of NCs

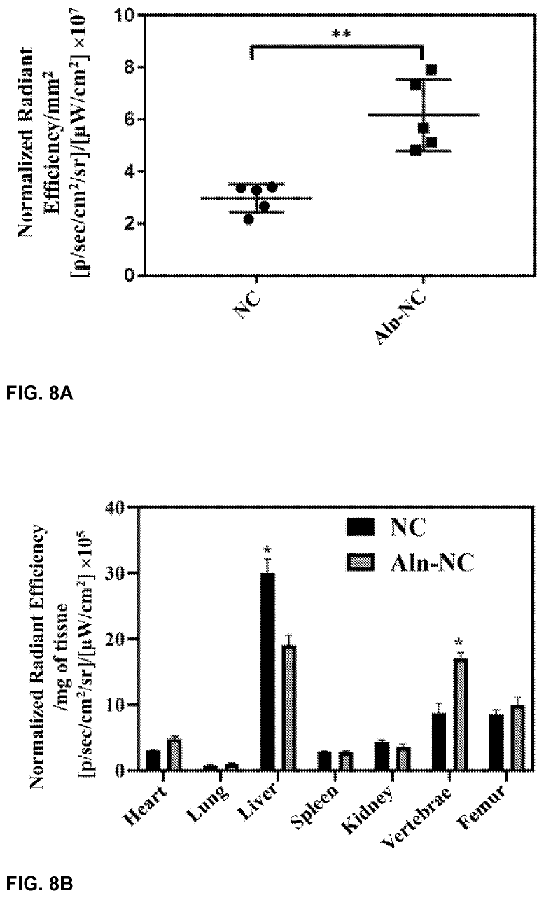

The ability of the nanocarriers to bind to bone tissue in vitro and in vivo was examined by using the Cy7 conjugated nanocarriers. The in vitro bone binding ability was assessed by incubating mouse bone chips with the Cy7 conjugated NC and Aln-NC for 2 hrs. The fluorescence intensity measurement showed significantly higher binding in bone chips incubated with Aln-NC compared to NC (FIG. 8A). In vivo distribution of the nanocarriers was examined by tracking the Cy7 labeled NCs and Aln-NCs through the use of an IVIS imaging system following tail vein injection into nude mice. IVIS imaging after 2 hrs post-injection showed fluorescence signal distributed throughout the body for both the nanocarriers. Time dependent imaging of the dorsal view region showed presence of Aln-NC in the vertebrae with significant fluorescent signaling at 6 hrs post administration. No such significant localization of nanocarrier to the vertebrae was noticed for the NCs 6 hrs post injection. A continuing increase in signal for Aln-NC was observed in the vertebrae for 3 days post-injection. The signal was found to decrease by day 4 and minimal to no signal was observed at day 14 for both the nanocarriers. The images of the ventral view showed optical signal from internal organs such as the liver and was observed for both the NCs and Aln-NCs, similar to prior studies. (Heller 2013). The ventral view images also showed that Aln conjugation of the nanocarrier decreases its accumulation in the liver.

The distribution of the nanocarriers within various organs was further evaluated by imaging liver, kidney, spleen, heart, lungs, vertebrae, femur, tibia, brain, pancreas, muscle, and skin following organ harvest at 72 hrs post-injection. Fluorescence imaging suggested accumulation of both nanocarriers within these organs. Between the Aln-NC and NC, Aln-NC showed significantly higher (˜45%) accumulation within the vertebrae (FIG. 8B). No significant difference was observed between the nanocarriers in their localization within the femur (FIG. 8B). Concomitant with the increase in the vertebrae, alendronate conjugation decreased nanocarrier accumulation within the liver (by ˜37%) and kidney (by ˜11%) compared to those lacking Aln groups (FIG. 8B).

The localization and distribution of the nanocarriers within the bone tissue was further examined by visualizing 10 um tissue sections of lumbar vertebrae and proximal femur, which showed key differences. Both NC and Aln-NC were found throughout the bone marrow. However, vertebral and femoral cross-sections showed localization of the Aln-NC at the marrow-to-bone interface, as well as in the bone marrow. In contrast, NC lacked such a localization and was present only in the bone marrow.

### Adenosine-Loaded Bone-Targeting Nanocarriers Prevent Bone Loss in Ovariectomized Mice

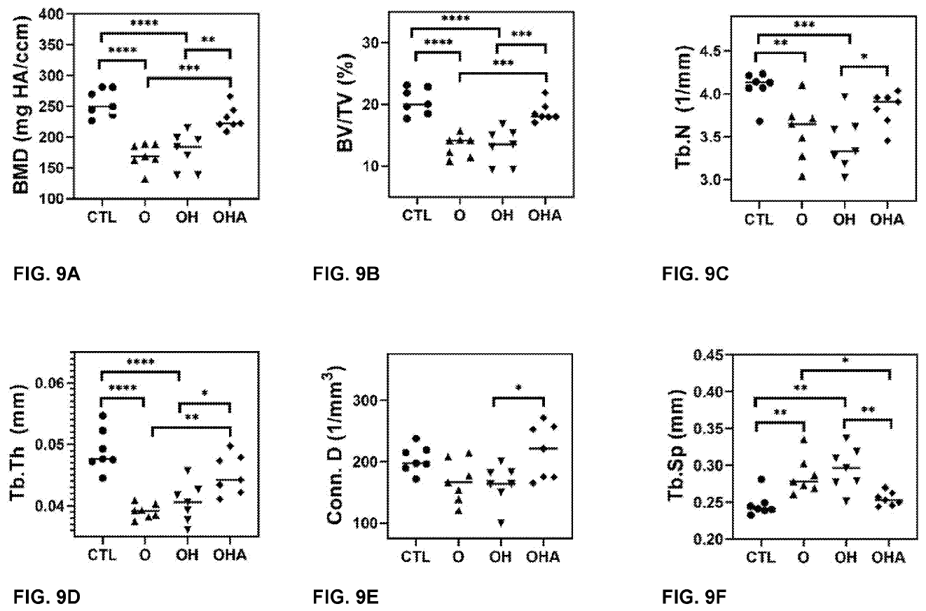

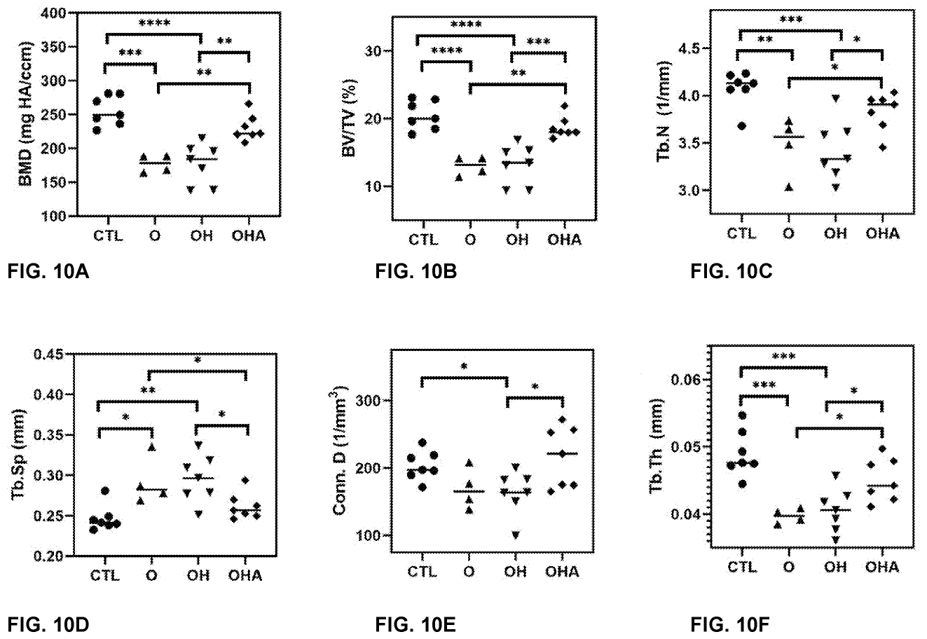

In view of the bone-targeting ability of Aln-NC, a mouse model of ovariectomy (OVX)-induced bone loss was used to evaluate the potential of using exogenous adenosine to treat bone degeneration. Four different groups—control healthy group (CTL), OVX without any treatment (O), OVX treated with Aln-NC (OH), and OVX treated with Aln-NC containing adenosine (OHA)—were studied. After 4 weeks post OVX surgery, the nanocarriers were administered twice a week, and bone tissues were characterized after 8 weeks of treatment. Care was taken to ensure that the mice received similar amounts (mg/kg of the body) of nanocarrier and Aln for OH and OHA groups. The dose of adenosine for the OHA group is ˜30 mg/kg of body weight. Ex vivo micro-computed tomography (μ-CT) was used to quantify the trabecular bone in the vertebrae. Quantification of the μ-CT images showed significant reduction in bone mineral density (BMD), bone volume ratio (BV/TV), trabecular number (Tb.N), trabecular thickness (Tb.Th), and connectivity density (Conn.D) with less trabecular spacing (Tb.Sp) for the OVX group (O) compared to the healthy group (CTL) (FIGS. 9A-9F). OVX mice treated with Aln-NC containing adenosine (OHA) showed significantly higher BMD, BV/TV, Tb.N, Tb. Sp, Tb.Th, and Conn.D compared to the OVX (O) (FIGS. 9A-9F). Furthermore, these trabecular bone parameters (BMD, BV/TV, Tb.N, Tb. Sp, Tb.Th, and Conn.D) were found to be similar to that of the healthy control (FIGS. 9A-9F). In contrast, OVX mice treated with Aln-NC (OH) showed trabecular bone morphology (BMD, BV/TV, Tb.N, Tb. Sp, Tb.Th, and Conn. D) similar to that of the OVX mice (O) (FIGS. 9A-9F). Similar trends were observed for the distal femur (FIGS. 10A-10F). Specifically, the adenosine treated group showed higher BMD, BV/TV, Conn.D, Tb.N, Tb.Th, and lower Tb.Sp compared to the OVX and Aln-NC groups (FIGS. 10A-10F).

The μ-CT data was further confirmed by histological analyses. Histomorphological changes of the lumbar vertebrae were examined by hematoxylin and eosin staining, which showed significantly more trabecular bone for the cohorts treated with OHA compared to O and OH groups. Histochemical staining for TRAP was used to detect the changes in osteoclast activity in the lumbar vertebrae. Cohorts treated with Aln-NC containing adenosine (OHA), showed lower levels of TRAP activity compared to the OVX (O) group and Aln-NC-treated group (OH).

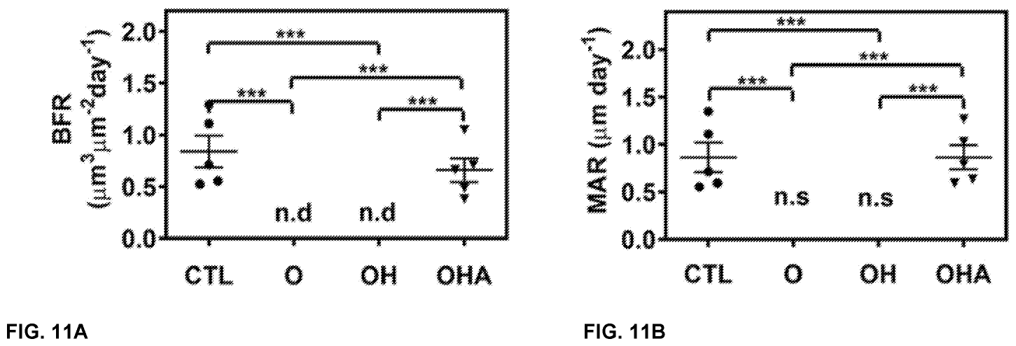

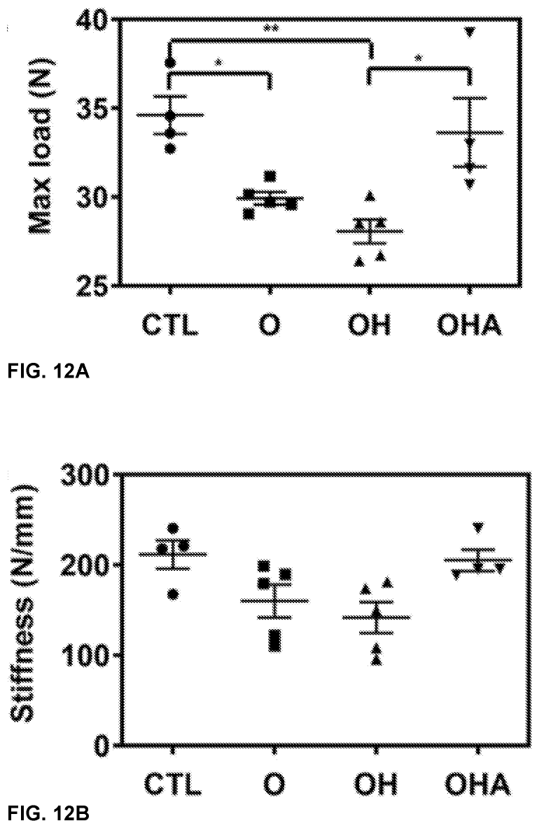

The contribution of new bone formation was assessed by double fluorescence labeling of mineral deposition with calcein, a green fluorescent dye, and alizarin, a red fluorescent dye, which were administered over a 9-day interval. Unlike healthy cohorts and cohorts treated with adenosine, no detectable separation between the dyes was observed for the OVX (O) and those treated with Aln-NC without adenosine (OH) groups. Histomorphometric analyses were used to quantify various parameters relevant to bone tissue formation such as bone formation rate (BFR, μm³/μm²/day) and mineral appositional rate (MAR; μm/day) (FIGS. 11A-11B). The BFR and MAR of OVX mice treated with Aln-NC containing adenosine (OHA) was significantly higher compared to those treated with Aln-NC without adenosine (OH) (FIGS. 11A-11B). Also, no significant difference in BFR and MAR could be observed between the adenosine treated group (OHA) and the healthy controls (CTL) (FIGS. 11A-11B). These findings were further corroborated through mechanical measurements of the bone tissue. Specifically, tibias of mice treated with Aln-NC containing adenosine (OHA) showed improved maximum load and stiffness compared to both OVX mice without any treatment (O) and mice treated with Aln-NC without adenosine (OH) (FIGS. 12A-12B).

### Discussion

This study demonstrated the therapeutic efficacy of adenosine to treat bone loss by developing a bone targeting nanocarrier for systemic administration of adenosine while mitigating its short half-life and off-target effects. Targeted delivery of therapeutics to tissues using nanocarriers has been shown to improve the bioavailability of the drugs while minimizing their off-target effects. (Rosenblum 2018; Jarvinen 2010; Hu 2015; Vanderburgh 2020). In addition, nanocarriers with their large surface area to volume ratio offer high drug-loading capacity, improved drug stability, and are promising drug delivery systems. (Zhang 2007; Rotman 2018; Zeng 2019; Sun 2016; Yang 2019). In fact, encapsulation of adenosine within the nanocarriers, such as liposomes, has been used to increase the longevity of adenosine in systemic administration, (Takahama 2009; Gaudin 2014). While the use of exogenous adenosine to treat ischemic injuries has been actively studied, the systemic delivery of adenosine to treat diseases such as osteoporosis has not been explored.

The hyaluronic acid nanocarrier was modified with functional groups to assist adenosine loading (via PBA molecules) and bone tissue binding (via Aln molecules). In a prior study, the ability of PBA molecules to bind reversibly to cis-diol groups to sequester adenosine molecules was used. (Zeng 2019). Harnessing the ability of Aln molecules to bind to bone apatite is an effective approach to target biomolecules to bone tissue. (Cheng 2017; Yin 2016). The high content of hydroxyapatite in the bone tissue provides a unique target for bone binding. Bisphosphonate molecules such as Aln, structural analogs to the endogenous pyrophosphate, are known to chelate with the calcium ions in the hydroxyapatite of bone extracellular matrix. (Cheng 2017; Yin 2016).

IVIS imaging examining the biodistribution of nanocarriers showed that both the non-functionalized and Aln-functionalized nanocarriers accumulated in the bone tissue (vertebrae, femur, tibia). While the NCs were found in multiple organs, such as liver, kidney and spleen, functionalization of nanocarriers with the Aln group significantly improved its ability to accumulate within the bone tissue. The presence of the nanocarriers at the bone-to-marrow interface was only observed for Aln-NC, further suggesting the Aln groups plays an important role in controlling the localization of the NCs. This finding could explain why vertebrae had more accumulation of Aln-NC than non-targeted NC, but no such differences in the case of the femur. Due to the cancellous nature, vertebrae has greater bone surface area which facilitates binding of the Aln-NC to the bone surface. On the other hand, no significant difference in the signal between Aln-NC and NC in femur could be due to the relatively large volume of femoral marrow along with the low surface area of the bone-to-marrow interface. (Heller 2013).

The systemic administration of adenosine by using Aln-NC significantly improved bone mineral density (BMD), bone volume, and other trabecular bone parameters, such as connectivity density and trabecular spacing, compared to the untreated OVX group and those treated with Aln-NC devoid of adenosine. Together, these results suggest that the observed beneficial effect of the systemic administration of the nanocarrier is solely due to adenosine. Despite the therapeutic use of Aln drugs for osteoporosis, its presence in the NCs did not contribute to prevention of bone loss. This could be due to the low amount of Aln present in the Aln-NCs, which only provides 2.4-2.8 mg Aln/kg of mice. In contrast, the therapeutic regimen involving Aln drugs commonly uses a dose of ˜35 mg/kg of mice. (Corral 1998; Chen 2014).

Osteoporotic bone loss is characterized by compromised osteoblast activity and excessive osteoclast activity. It has been shown that exogenous adenosine promotes osteoblastogenesis while decreasing osteoclastogenesis. (Shih 2019; Mediero 2013; Kang 2016; Mediero 2012). Previously, it was shown that exogenous adenosine mediated osteoblastogeneis and osteoclastogenesis involves A2BR signaling. (Shih 2019). The dual ability of adenosine molecule to promote osteoblastogenesis and inhibit osteoclastogenesis explains why adenosine treatment of OVX mice resulted in significantly improved bone mass, trabecular features, and mechanical properties comparable to the healthy control.

### Conclusions

In summary, a bone targeting nanocarrier was developed for systemic administration of adenosine. The nanocarrier, composed of hyaluronic acid and phenylboronic acid, was synthesized via the emulsion suspension polymerization method. The loading and release of adenosine was achieved by harnessing the ability of boronate molecules to foini dynamic covalent bonds with cis-diol molecules such as adenosine. The ability of alendronate groups to bind to hydroxyapatite was used to promote its localization within the bone tissue. Systemic administration of alendronate functionalized nanocarriers encapsulated with adenosine effectively prevented bone loss in osteoporotic mice. Furthermore, adenosine treatment via the bone targeting nanocarrier promoted new bone formation and improved bone mechanical strength. Together, the results show that systemic administration of exogenous adenosine is a therapeutic strategy to treat osteoporosis and promote bone health.

### Example 2

### Clickable Microgel-Based Injectable Scaffold for Bone Tissue Engineering

### Introduction

Promoting tissue repair by harnessing the endogenous stem and progenitor cells with the help of biomaterials offers great potential in regenerative medicine. Herein, intrinsically porous scaffolds have been developed for the delivery of therapeutic molecules to engineer bone tissue. Microgels (MGs), consisting of a naturally occurring polymer hyaluronic acid and phenyl boronic acid (PBA), were used as the building blocks of the scaffold. The MGs were synthesized via photopolymerization of hyaluronic acid methacrylate (HA-MA) and 3-acrylamido-phenyl boronic acid (3A-PBA) in a microfluidic device using water-in-oil emulsion. The gel particles were designed with mean diameters of ˜100 μm. Strain-promoted clickable functional groups (dibenzocyclooctyne (DBCO) and azide groups) were then introduced on the surface of the microgel via chemical conjugations of DBCO-PEG4-amine and azido-PEG4-amine respectively. Particles were then assembled to create porous scaffold with or without adenosine, an osteoanabolic molecule, upon mixing. HA polymers containing DBCO and azide groups were also used as dopants to form a stable porous scaffold. This microgel based system provides the opportunity to regulate several aspects, such as controlled adenosine delivery, cellular growth and differentiation, along with the ability to recapitulate biological interfaces.

### Materials and Methods

### Materials

Hyaluronic acid (molecular weight 40 kDa, HA40K-5) was purchased from Lifecore, USA. Methacrylic anhydride (276685), N-hydroxysuccinimide (NHS, 130672), sodium hydroxide (795429), adenosine (A4036) and mineral oil (M5904) were obtained from Millipore Sigma, USA. 1-Ethyl-3-(3-dimethylaminopropyl) carbodiimide hydrochloride (EDC, D1601) and Azido-PEG4-amine (A3004) were obtained from TCI Chemicals, USA. DBCO-PEG4-Arnine (A103P) was obtained from Click Chemistry Tools, USA. Dialysis bag (molecular weight cut off 3.5 kDa) was obtained from Spectrum, USA. ABIL EM90 smfactant (420095-L-151) was obtained Universal Preserv-A Chem INC, Germany. n-Hexane, acetone, ethanol, dimethyl sulfoxide (DMSO) were purchased from Millipore-Sigma, USA and were off ACS or spectroscopic grade.

### Synthesis of Hyaluronic Acid Methacrylate (HA-MA)

HA-MA was synthesized by reacting HA with methacrylic anhydride. Briefly, HA (600 mg) was dissolved into deionized (DI) water. Methacrylic anhydride (4.4 mL) was added to the HA solution and the pH of the reaction mixture was adjusted to 8-8.5 by adding 5 N NaOH. The reaction was continued for about 24 h at 4° C. Excess of ice-cold ethanol-acetone mixture (1:1) was added to precipitate the polymer. The precipitate was filtered, washed several times with ice-cold EtOH-acetone mixture. Next, the polymer was dissolved in DI water and dialyzed for 3 days (using 3.5 kDa membrane) against DI water. The solution was then freeze dried to obtain the methacrylated HA. The polymer was characterized via a combination of FTIR and ¹HNMR spectroscopy.

The degree of methacrylation was 35±5%.

### Synthesis of DBCO-Conjugated HA (I-IA-DBCO)

HA (200 mg) was dissolved in 1:1 water-DMSO mixture at 10 mg/mL. Solid EDC (192 mg) and NHS (115 mg) were gradually added to HA solution at 15 min intervals. After 30 min, DBCO-PEG4-amine (261 mg) was added to reaction mixture. The reaction was continued for about 48 h at room temperature. The mixture was then dialyzed in 10 mM NaCl for 4 days and DI water for 1 day. Finally, the solution was freeze-dried to obtain HA-DBCO. The polymer was characterized via a FTIR and ¹HNMR spectroscopy.

The degree of DBCO-PEG4-amine conjugation was found to be ˜11%.

### Synthesis of Azide-Conjugated HA (HA-Azide)

HA (200 mg) was dissolved in IVIES buffer of pH 5.5 at 10 mg/mL. Solid EDC (192 mg) and NHS (115 mg) were gradually added to HA solution at 15 min intervals. After 30 min, Azido-PEG4-amine (109 mg) was added to reaction mixture. The reaction was continued for about 48 h at room temperature. The mixture was then dialyzed in 10 mM NaCl for 3 days and DI water for 1 day. Finally, the solution was freeze-dried to obtain HA-Azide. The polymer was characterized via a FTIR and ¹HNMR spectroscopy.

The degree of azide-PEG4-amine conjugation was ˜13%.

### Micro Gel Fabrication

The HA-PBA micro gels were fabricated using water-in-oil emulsion method in a two inlet, one outlet microfluidic device. Briefly, one inlet was used for the ‘inner pinching’ oil (10% v/v ABIL EM 90 in mineral oil) while the other inlet allowed the solution of HA-MA and 3A-PBA with the photoinitiator LAP. The outlet just consisted of oil (10% v/v ABIL EM 90 in mineral oil). The HA solution was prepared by dissolving HA-MA in DI water at 5% w/v. 3A-PBA was dissolved in ethanol at 9.25% w/v. The two solutions were then mixed together. Next, LAP was added to the HA-PBA mixture at 0.1% w/v. The solution was then loaded into the 1 mL Hamilton Syringe. The gel precursor solution was then co-flowed with the oil at 1:1 volume to form microgel droplets and simultaneously cross linked with UV. The microgels were transferred to Eppendorf tubes and centrifuged at 10000 rpm for 10 min. The supernatant was discarded and the microgels were successively washed with n-hexane (3 times), isopropanol (2 times) and fmally with water (1 time) to remove all the oil and surfactant. Finally, the particles were freeze dried.

Stable micro gel was formed using the microfluidic techniques. The size of the microgel was 100±10 μm.

### Surface Functionalization of Micro Gel

The microgel-surface was functionalized with clickable groups following amide coupling. Briefly, freeze dried micro gel was suspended in water for azide coupling or in a 1:1 water-DMSO mixture for DBCO coupling. Solid EDC (3 equivalent with respect to carboxylic acid group in micro gel) and NHS (4.5 equivalent with respect to carboxylic acid group in microgel) were added to the microgel suspension at 15 min intervals. After 30 min, Azido-PEG4-amine or DBCO-PEG4-amine (1 equivalent with respect to carboxylic acid group in microgel) was added to reaction mixture. The reaction was continued for about 48 h at room temperature. After the reaction, micro gels were centrifuged at 10000 rpm for 10 min and washed repeatedly (3 times) with water to remove unreacted reagents and finally freeze dried. The surface functionalization was confirmed via a combination a UV-visible, FTIR and ¹HNMR spectroscopy.

The degree of DBCO functionalization on the HA-PBA microgel surface was ˜5-6% while the degree of azide functionalization was ˜7%.

### Preparation of the Scaffold

The scaffold was prepared upon mixing the DBCO and azide modified microgels. Briefly, both the DBCO and Azide modified microgels were suspended in water at 10% w/v. HA-DBCO polymer was added to the DBCO-modified microgel suspension at 0.5 wt % whereas HA-Azide polymer was added to the Azide modified microgel suspension at 0.5% w/v respectively. Finally, the two microgel suspensions were mixed together to form the scaffold.

### Example 3

### Dysregulation of Ectonucleotidase-Mediated Extracellular Adenosine During Postmenopausal Bone Loss

### Introduction

Adenosine and its receptors play a key role in bone homeostasis and regeneration. Extracellular adenosine is generated from CD39 and CD73 activity in the cell membrane, through conversion of adenosine triphosphate to adenosine monophosphate (AMP) and AMP to adenosine, respectively. Despite the relevance of CD39/CD73 to bone health, the roles of these enzymes in bona fide skeletal disorders remain unknown.

More particularly, emerging studies suggest the pivotal role played by naturally occurring purinergic nucleoside adenosine and its signaling in bone tissue formation, function, and repair (Kara 2010; Katebi 2009; Carroll 2012). There has been a surge in research activity to understand the role of adenosine receptors, including the A1 receptor (A1R), A2AR, and A2BR, in bone tissue formation and maintenance (Kara 2010; Katebi 2009; Carroll 2012; He 2012; Gharibi 2011). Studies have shown that A2AR and A2BR activation promotes osteogenic differentiation of osteoprogenitors (Katebi 2009; Carroll 2012; Mediero 2012; Shih 2014) and inhibits osteoclastogenesis (Mediero 2012; Mediero 2016; Mediero 2018; Corciulo 2016). Recently, it was shown that extracellular phosphate uptake by the SLC20a1 phosphate transporter on the cell membrane supports osteogenesis of mesenchymal stem cells (MSCs) via adenosine, which acts as an autocrine/paracrine signaling molecule through the A2BR (7). Activation of the A2BR also inhibited adipogenesis of human MSCs (Kang 2015). These findings suggest the possibility of dysregulation of extracellular adenosine and its signaling during bone disorders.

CD39 (ectonucleoside triphosphate diphosphohydrolase-1) and CD73 (ecto-5′-nucleotidase) are membrane-bound ectonucleotidases that regulate extracellular adenosine by hydrolyzing extracellular adenosine triphosphate to adenosine diphosphate and adenosine monophosphate (AMP) and AMP to adenosine, respectively (Dwyer 2007; Yegutkin 2014). These ectonucleotidases are well known for their immunosuppressive functions (Deaglio 2007) and affect a variety of pathophysiological events, including but not limited to autoimmune diseases (Fletcher 2009; Tai 2013), infections (Raczkowski 2018; Alam 2014), atherosclerosis (Kanthi 2015; Buchheiser 2011), ischemia-reperfusion injury (Köhler 2007; Hart 2008), cancer (Sun 2010), and transplant tolerance (Deaglio 2007). Recent studies have demonstrated the importance of these enzymes in bone health: Mice lacking CD73 develop osteopenia with impaired osteoblast function (Takedachi 2012) and the activity of CD73 in osteoblasts is essential to bone repair in aged mice (Bradaschia-Correa 2017). Despite these studies implying the role of ectoenzymes on bone tissue formation and osteogenic commitment of progenitor cells (Takedachi 2012; He 2013), their role in bona fide skeletal disorders such as postmenopausal osteoporosis remains unknown.

Osteoporosis is a condition of severe bone loss affecting 10 million individuals above 50 years of age and inflicting 2 million fractures each year (U.S. Office of the Surgeon General 2004; Burge 2007). Dynamic bone remodeling, dictated by the balance between osteoblast and osteoclast functions that contribute to bone formation and bone resorption, respectively, is crucial to maintain bone health (Eastell 2016). During postmenopausal osteoporosis, reduced production of estradiol (E2) disturbs bone homeostasis due to altered estrogen receptor (ER) signaling, resulting in decreased osteoblast (Zhou 2001; Chang 2009) and increased osteoclast activities (Eghbali-Fatourechi 2003). Several mechanisms have been proposed for the regulation of ERs on osteoblast and osteoclast functions, such as cross-talk and synergy of ER signaling with osteogenic signaling, including Wnt/β-catenin (Almeida 2013) and transforming growth factor-β (Hawse 2008). During osteoclast differentiation, estrogen blocks RANKL/M-CSF (receptor activator of nuclear factor κB ligand/macrophage colony-stimulating factor)-induced activator protein-1-dependent transcription, likely through direct regulation of c-Jun activity (Shevde 2000), and ESR1 promotes apoptosis of osteoclasts via the induction of the Fas/Fas ligand system (Nakamura 2007).

The expression levels of ectonucleotidases CD73 and CD39 during osteoporosis and the effects of altered activity of these enzymes on extracellular adenosine levels were examined. Knockdown of estrogen receptors ESR1 and ESR2 in primary osteoprogenitors and osteoclasts undergoing differentiation showed decreased coexpression of membrane-bound CD39 and CD73 and lower extracellular adenosine. A direct correlation between impaired ectonucleotidase expression and extracellular adenosine levels in a mouse model of postmenopausal bone loss was demonstrated. Given the importance of estrogen in postmenopausal bone loss, the role of ER signaling on ectonucleotidase expression and extracellular adenosine levels was established, with a focus on A2BR signaling. Targeting the adenosine A2B receptor using an agonist attenuated bone loss in ovariectomized mice. In particular, in an experiment yielding clinical implications, the A2BR agonist BAY 60-6583 was used to compensate for the decrease in adenosine signaling and show that this intervention attenuates bone loss. Together, these findings suggest a pathological association of purine metabolism with estrogen deficiency and highlight the potential of A2B receptor as a target to treat osteoporosis.

### Materials and Methods

### Animal Experiments

Female C57BL6/J mice were used (the Jackson laboratory, Bar Harbor, Me.). All animal studies were performed with the approval of the Institutional Animal Care and Use Committee at Duke University and in accordance to guidelines of National Institutes of Health All tools were sterile-autoclaved before use.

### Ovariectomy Surgery

Ovariectomy surgeries were performed at the Jackson laboratory or in-house at 12 weeks old as previously described (Idris 2012). Before in-house ovariectomy surgery, animals were anesthetized with isoflurane (Henry Schein, Dublin, Ohio) by inhalation at 1 to 3% induction and 4% maintenance and administered with buprenorphine SR (Zoopharm, Windsor, Colo.) subcutaneously. A 3 cm by 3 cm of area cephalic from the iliac crest on left and right side of mice was shaved and wiped with 10% povidone-iodine (Purdue Products, Stanford, Conn.). A 2- to 3-cm midline incision was made, and the skin was bluntly dissected from the underlying fascia. Another incision was made through the fascia, 1 cm lateral of the midline, and bluntly dissected laterally until it reaches the abdominal cavity. The adipose tissue that surrounds the ovary in the abdominal cavity was gently pulled out by tweezers. The uterine horns and vessels were ligated 0.5 to 1 cm proximally, and the ovary was cut. The fascia wound was closed using a degradable vicryl 5-0 suture (Ethicon, Somerville, N.J.), and the skin wound was closed with a 3-0 nylon suture (Ethicon) with a topical application of 0.5% bupivacaine (Hospira, Lake Forest, Ill.). Another incision in the contralateral fascia was performed, and the procedure was repeated. Animals were monitored for the duration of the surgery.

### Administration of BAY 60-6583

BAY 60-6583 (Tocris Bioscience, Minneapolis, Minn.) was dissolved in dimethyl sulfoxide (DMSO; Sigma-Aldrich, St. Louis, Mo.). For in vivo administration, fresh solutions of BAY 60-6583 (10%, v/v), 0.9% sodium chloride (NaCl; 50%, v/v; Hospira), and polyethylene glycol 400 (PEG 400; 40%, v/v; Thermo Fisher Scientific, Hampton, N.H.) were mixed and sterile-filtered through 0.22-μm-pore filter disk, and 100 μl of solution was injected intraperitoneally at mouse weight (1 mg/kg), after 8 weeks of ovariectomy surgery, once every 2 days. A solution comprising DMSO (10%, v/v), 0.9% NaCl (50%, v/v), and PEG 400 (40%, v/v) was injected in animals as vehicle control.

### Cell Isolation, Culture, and Differentiation

Three- to 4-week-old female C57BL/6 J mice were euthanized by carbon dioxide and bilateral thoracotomy.

### Isolation of Osteoprogenitor Cells and Culture

Osteoprogenitor cells were isolated as previously described with modifications. All buffers were ice cold, unless otherwise indicated. Briefly, the femur, tibia, humerus, radius, and ulna of mice were harvested. BM was flushed out and discarded. The bone was collected and cut into 1-mm³ chips in harvest buffer and then digested in digestion buffer containing growth media [α minimum essential medium (αMEM), fetal bovine serum (FBS) (10%, v/v), penicillin/streptomycin (10,000 U/ml; 1%, v/v)], and collagenase type 2 (1 mg/ml, w/v; Worthington Biochemical, Lakewood, N.J.) while shaking at 60 rpm on orbital shaker (catalog no. 51700-13, Cole-Parmer, Vernon Hills, Ill.) in humidified incubator (37° C., 5% CO₂) for 1.5 hours. Digested bone chips were rinsed three times with growth media and transferred to two wells of six-well plate for culture. Media were replaced after 3 days, and bone chips were further cultured for 3 days for cells to adhere and proliferate before passage. For the first passage, cells were treated with 0.25% trypsin-EDTA (Thermo Fisher Scientific) for 3 min, neutralized with growth media, detached with cell scraper, and subcultured along with bone chips. The procedure for subsequent passages were similar but without bone chips. All experiments were performed at three to four passages. For estradiol (E2) withdrawal experiments, cells were cultured in the absence or presence of E2 (Sigma-Aldrich) supplemented at 100 nM every 3 hours to charcoal-stripped growth media [αMEM, charcoal-stripped FBS (10%, v/v), penicillin/streptomycin (10,000 U/ml; 1%, v/v)]. For adenosine supplementation, cells were supplemented with adenosine (30 μg/ml; Sigma-Aldrich) in growth media with fresh changes of media every day.

### Isolation of Mononuclear Cells and Macrophage/Osteoclast Differentiation

All buffers used were ice cold, unless otherwise indicated. Briefly, the femur, tibia, humerus, radius, ulna, and vertebra were harvested and crushed with pestle and mortar in harvest buffer [phosphate-buffered solution (PBS) and FBS (2%, v/v)] to release BM tissue. BM was passed through a 70-μm cell strainer and centrifuged at 200 g for 5 min. Cells were resuspended in harvest buffer, gently layered onto Ficoll-Paque PLUS (GE Healthcare, Marlborough, Mass.) at 1:1 ratio, and centrifuged without rotor acceleration and deceleration at 200 g for 15 min. Afterward, the opaque middle layer with cells was collected, washed with harvest buffer, and centrifuged at 200 g for 5 min to yield a cell pellet. Isolated mononuclear cells were cultured in macrophage induction media, containing growth media, prostaglandin E₂ (PGE₂; 10⁻⁷ M; Santa Cruz Biotechnology, Dallas, Tex.), and M-CSF (10 ng/ml; PeproTech, Rocky Hill, N.J.) at 100,000 cells/cm². For osteoclast differentiation, macrophages cultured for 3 days were further induced in osteoclast induction media containing growth media, PGE2 (10⁻⁷ M), M-CSF (10 ng/ml), and RANKL (10 ng/ml; PeproTech). For estradiol (E2; Sigma-Aldrich) withdrawal experiments, osteoclasts were cultured in the absence or presence of E2 supplemented at 100 nM to media containing charcoal-stripped growth media, PGE2 (10⁻⁷ M), M-CSF (10 ng/ml), and RANKL (10 ng/ml; PeproTech). For adenosine supplementation, cells were supplemented with adenosine (30 μg/ml; Sigma-Aldrich) in differentiation media with fresh media change every day.

siRNA Knockdown

Cells were transfected with Silencer Select siRNA oligonucleotides (Thermo Fisher Scientific), according to the manufacturer's instructions. Briefly, 5 nM siRNA was mixed with RNAiMAX transfection reagent (Thermo Fisher Scientific) in the presence of Opti-MEM (Thermo Fisher Scientific) for 5 min at room temperature (RT). The solution was transfected into osteoprogenitor cells in the presence of growth media or mononuclear cells in macrophage induction media for 2 days. The following siRNA oligonucleotides were used: ADORA2B (A2BR; catalog no. 4390771; ID, s62047), ESR1 (catalog no. 4390771; ID, s65686), ESR2 (catalog no. 4390771; ID, s65689), and negative control #1 (catalog no. 4390843). A concentration of 5 nM negative control siRNA was used for single ER knockdown, while a concentration of 10 nM negative control siRNA was used for dual ER knockdown.

### Flow Cytometry

Whole BM flush were collected from tibia and femur into buffer containing PBS [3% (v/v) bovine serum albumin (BSA)] and filtered through 40-μm nylon filter (BD Biosciences, San Jose, Calif.). Red blood cells (RBCs) were lysed with RBC lysis buffer (Thermo Fisher Scientific) for 5 min at RT. Cells were stained with CD39 phycoerythrin (PE)/cyanine-7 (8 μg/ml; 143805, BioLegend, San Diego, Calif.), CD73 PE (1.25 μg/ml; 12-0731-82, Thermo Fisher Scientific), and CD45 allophycocyanin (APC) (1.25 μg/ml; 17-0451-82, Thermo Fisher Scientific) antibodies for 30 min at RT. Stained cells were analyzed with BD Accuri C6 flow cytometer and CFlow software. Analyses were performed by comparing to unstained cells and gated for singlets.

### RNA Isolation, Reverse Transcription, and Real-Time Polymerase Chain Reaction

Total RNA was extracted with TRIzol (Thermo Fisher Scientific), phase-separated with chloroform, and precipitated using isopropanol. One microgram of RNA was reverse-transcribed using iScript cDNA Synthesis Kit (Bio-Rad), according to the manufacturer's instructions. iTaq Universal SYBR green reagent (Bio-Rad) was used to detect gene expression during amplification of complementary DNA after initial denaturation at 95° C. for 30 s for one cycle and 95° C. for 5 s and 60° C. for 30 s for 40 cycles on a polymerase chain reaction (PCR) cycler (Bio-Rad). The mouse primer sequences are as follows:

OSX
(forward, TGCCTGACTCCTTGGGACC (SEQ ID NO: 01);
reverse, TAGTGAGCTTCTTCCTCAAGCA (SEQ ID NO: 02)),
OPN
(forward, AAACCAGCCAAGGTAAGCCT (SEQ ID NO: 03);
reverse, TCAGTCACTTTCACCGGGAG (SEQ ID NO: 04)),
NFATC1
(forward, GGTAACTCTGTCTTTCTAACCTTA (SEQ ID NO : 05);
reverse, GTGATGACCCCAGCATGCACCAGTCACAG (SEQ ID NO: 06)),
CTSK
(forward, GGGCTCAAGGTTCTGCTGC (SEQ ID NO: 07);
reverse, TGGGTGTCCAGCATTTCCTC (SEQ ID NO: 08)),
ACP5
(forward, CAGCAGCCCAAAATGCCT (SEQ ID NO: 09);
reverse, TTTTGAGCCAGGACAGCTGA (SEQ ID NO: 10)),
ESR1
(forward, CTTGAACCAGCAGGGTGGC (SEQ ID NO: 11);
reverse, GAGGCTTTGGTGTGAAGGGT (SEQ ID NO: 12)),
ESR2
(forward, GACGAAGAGTGCTGTCCCAA (SEQ ID NO: 13);
reverse, GCCAAGGGGTACATACTGGAG (SEQ ID NO: 14)),
ADORA2B
(forward, ATCTTTAGCCTCTTGGCGGTG (SEQ ID NO: 15);
reverse, GACCCAGAGGACAGCAATGAT (SEQ ID NO: 16)),
and
18S ribosomal RNA
(forward, ACCAGAGCGAAAGCATTTGCCA (SEQ ID NO: 17);
reverse, ATCGCCAGTCGGCATCGTTTAT (SEQ ID NO: 18)).

### Adenosine Assay

Adenosine levels were measured from plasma of BM flush or cultured media using adenosine assay kit (Abcam), according to the manufacturer's instructions. For BM measurements, femur and tibia containing marrow were centrifuged at 200 g for 1 min at 4° C. to collect whole-marrow flush. The flush was then centrifuged at 2000 g for 5 min at 4° C. to separate the cell and plasma fractions. For cell media measurements, media cultured with cells for 3 days were collected. The BM plasma or cell media samples were then diluted and mixed with reaction mix containing adenosine assay buffer, adenosine detector, adenosine converter, adenosine developer, and adenosine probe at 1:1 ratio in a well of 96-well white plate. Fluorescence intensity was detected with a plate reader (Tecan Infinite 200 PRO) using Ex535 and Em590 nm filters. Fluorescence was subtracted from background and quantified using a standard.

### Enzyme-Linked Immunosorbent Assay

Plasma estradiol from mouse peripheral blood was quantified using estradiol assay kit (R&D Systems, Minneapolis, Minn.), according to the manufacturer's instructions. Briefly, murine peripheral blood was collected from tail vein in the presence of heparin and centrifuged at 1000 g for 10 min at 4° C. to separate the cell and plasma fractions. Samples were pretreated with pretreatment E solution and centrifuged, and supernatant was mixed with pretreatment F solution. To wells coated with estradiol antibody, samples and estradiol conjugate were added and incubated for 2 hours at RT on a shaker. Wells were washed, and substrate solution was added for 30 min at RT and protected from light. Then, stop solution was added, and measurements were performed with a plate reader (Tecan Infinite 200 PRO) using Ex450 and Em540 nm filters.

### Histology and Staining

Vertebral samples were fixed with 4% paraformaldehyde (PFA) at 4° C. for 1 day and decalcified using 10% EDTA (pH 7.3) for 2 weeks at 4° C. The samples were gradually dehydrated using increasing concentrations of ethanol and incubated in CitriSolv (Decon Labs, King of Prussia, Pa.) until equilibrium was reached. Following dehydration, samples were immersed in a mixture of 50% (v/v) CitriSolv (Decon Labs) and 50% (w/w) paraffin (General Data Healthcare) for 30 min at 70° C. The samples were embedded in paraffin and sliced into sections of 10-μm thickness using a rotary microtome (RM2255, Leica). Before staining, the sections were deparaffinized using CitriSolv and subsequently rehydrated with decreasing concentration of ethanol until the samples were equilibrated with deionized (DI) water.

### H&E Staining

H&E staining was performed by first incubating the samples in hematoxylin solution (RICCA Chemical, Arlington, Tex.) for 1 min, followed by incubation with Eosin-Y solution (Richard-Allan Scientific, San Diego, Calif.) for 20 s. Stained sections were gradually dehydrated using increasing concentrations of ethanol until equilibrium was reached. Sections were mounted in glycerol and imaged using a Keyence BZ-X700 microscope.

### TRAP Staining

TRAP staining was performed using TRAP kit following the manufacturer's instructions (Sigma-Aldrich). Briefly, the solution was prepared by first mixing 50 ml of Fast Garnet GBC base solution with 50 ml of sodium nitrite solution. This mixture was added to 4.5 ml of DI water prewarmed at 37° C. After mixing, 50 ml of Naphthol AS-B1 phosphate solution, 200 ml of acetate solution, and 100 ml of tartrate solution were added to the solution and mixed to generate a working solution. Rehydrated sections were immersed in the working solution, incubated at 37° C. for 1 hour covered from light, and rinsed with ultrapure water. Sections were then gradually dehydrated using increasing concentrations of ethanol until equilibrium was reached. Slides were mounted with glycerol and imaged immediately. Images were quantified with ImageJ for TRAP⁺ cells and normalized to the length of bone surfaces.

### Immunofluorescence Staining

For immunofluorescence staining, rehydrated sections were immersed in a solution of proteinase K (20 mg/ml; Thermo Fisher Scientific) in 95% (v/v) TE buffer [50 mM tris-HCl, 1 mM EDTA, and 0.5% (v/v) Triton X-100 (pH 8)] with 5% (v/v) glycerol and incubated for 15 min at 37° C. Sections were washed with PBS and permeabilized using 0.1% Triton X-100 in PBS for 10 min at RT. The sections were immersed in a blocking solution [1% (w/v) BSA, glycine (0.25 M), normal donkey serum (5%, v/v), and normal goat serum (5%, v/v) in tris-buffered solution (TBS)] and incubated for 1 hour at RT. Sections were then incubated with primary antibody against CD39 (5 μg/ml; AF4398, R&D Systems), CD73 (5 μg/ml; AF4488, R&D Systems), and A2BR (1:200; MBS8207549, MyBioSource, San Diego, Calif.) in diluent solution (1%, w/v) and normal donkey serum (1%, v/v) in TBS overnight at 4° C. Sections were stained with secondary antibody using anti-donkey or anti-goat Alexa Fluor 647 (1:250; Jackson ImmunoResearch, West Grove, Pa.). For immunocytochemical costaining, cells were incubated with primary antibody against CD39 (1:100; ab227840, Abcam, Cambridge, UK) and CD73 (5 μg/ml; AF4488, R&D Systems) and stained with secondary antibody using anti-donkey Alexa Fluor 488 and anti-goat Alexa Fluor 647 (1:250; Jackson ImmunoResearch). Images were acquired and presented as pseudocolors.

### Microcomputed Tomography

Bone mineralization was analyzed as previously described (Shih 2017). L3 to L5 vertebra and femur were collected, fixed in 4% PFA at 4° C. for 1 day, and rinsed thoroughly with PBS. The fixed samples were placed in a 50-ml centrifuge tube with styrofoam spacers and loaded into a microCT scanner (vivaCT 80, Scanco Medical, Wayne, Pa.) and scanned at 55 keV at a pixel resolution of 10.4 μm. The reconstruction of scanned images was performed using microCT Evaluation Program V6.6 (Scanco Medical), followed by generation of radiographs and three-dimensional models using microCT Ray V4.0 (Scanco Medical). Mineral density of the scaffolds was quantified and presented as a percentage of BV/TV based on 100 contiguous slices.

### Bone Labeling

Animals were administered with calcein (10 mg/kg body weight; Sigma-Aldrich) at 14 days and alizarin complexone (30 mg/kg body weight; Sigma-Aldrich) at 5 days before euthanization. Collected cranium were fixed in 4% PFA at 4° C. for 1 day and stored in 70% ethanol. Subsequently, the samples were dehydrated at 70% ethanol (2 days), 95% ethanol (2 days), 100% 2-propanol (twice for 1 day), and xylene (twice for 2 days). After dehydration, the samples were infiltrated with methyl methacrylate embedding mixture, sectioned, and imaged.

### Mechanical Measurement

Mechanical properties of 12 tibiae per group were measured as previously described. After removing soft tissues, tibia samples were wrapped in wet tissue and frozen at −20° C. Sixteen hours before testing, the samples were transferred to 4° C. and then to RT an hour before testing. The four-point bending mode of ElectroForce 3220 (TA Instruments, New Castle, Del.) instrument with 225-N load cell was used. Samples were aligned on the fixtures in a manner that the load was applied perpendicular to the principal axis of tibia. The span length of the bottom support was 9.2 mm, while the top span length was 2.8 mm. Bending test was performed in displacement control mode at a rate of 0.025 mm/s. Load-displacement data were recorded at a data acquisition rate of 10 Hz. Displacement was tared at the first data point at which the load equaled or exceeded 1 N. Maximum load is the highest load (newtons) before the sample fractures. Bending stiffness (newtons per millimeter) was calculated as the slope of load versus displacement between 30 and 70% of maximum load to failure in the linear region.

### Statistical Analyses

Statistical analyses were carried out using GraphPad Prism 5. Two-tailed Student's t test was used to compare two groups. One-way analysis of variance (ANOVA) with Tukey post hoc test was used to compare three or more groups. The P values were obtained from each test.

### Results

### Surface Membrane Ectonucleotidases and Extracellular Adenosine are Decreased in Bone Marrow of Osteoporotic Animals

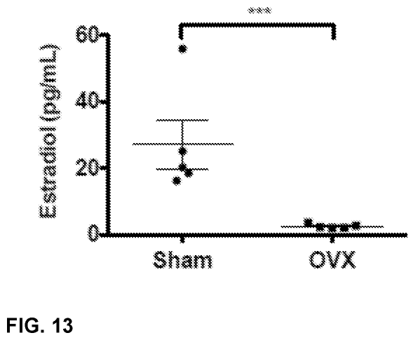

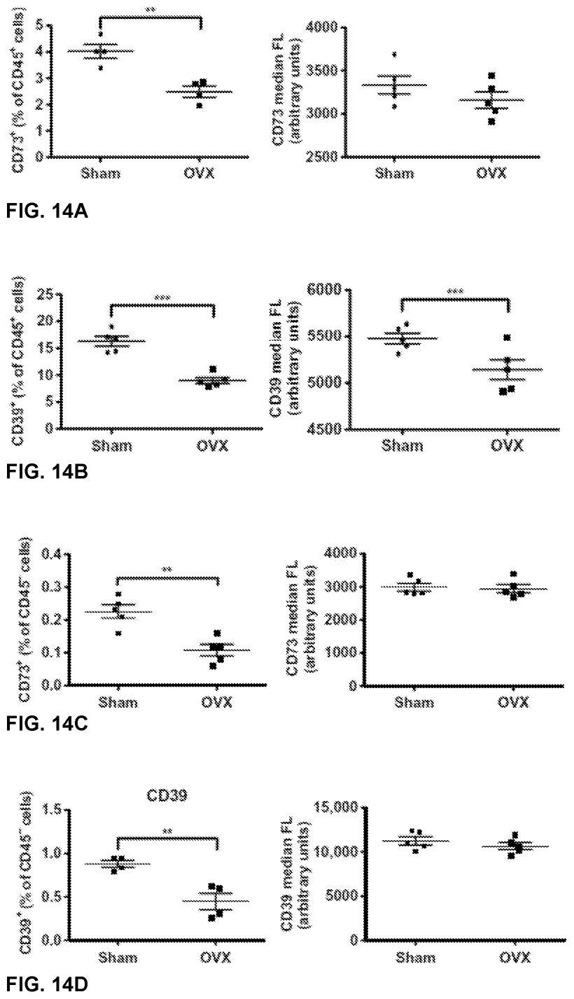

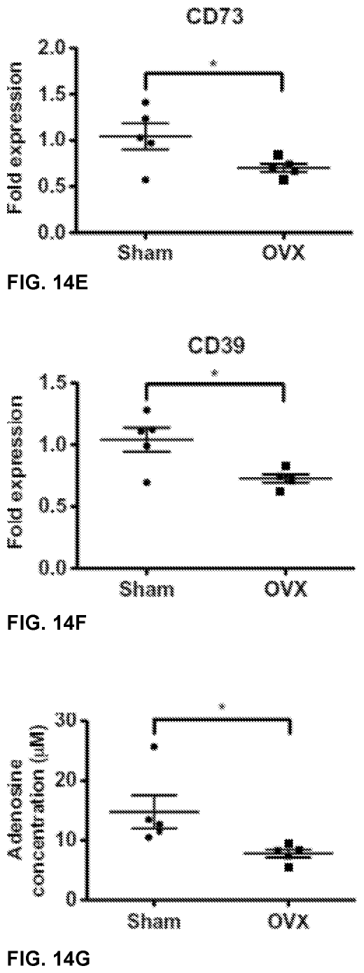

In this study, ovariectomized (OVX) mice were used; they are widely recognized as a model of postmenopausal osteoporosis (Kalu 1999). Estradiol measurements in the peripheral blood and microcomputed tomography (microCT) analyses of the vertebrae after 4 weeks of ovariectomy were used to ensure bone loss (FIG. 13). Analyses of CD73 and CD39 on OVX bone surfaces revealed a decreased expression compared to sham control. Next the levels of these ectonucleotidases in the hematopoietic and nonhematopoietic fraction of bone marrow (BM) cells was examined. Quantification of the flow cytometric analyses of hematopoietic cells (FIGS. 14A-14B) demonstrated a significantly lower fraction of cells expressing CD73 and CD39, as well as decreased median fluorescence for these ectonucleotidase compared to the control. Analyses of the nonhematopoietic cells showed a similar observation that a significantly lower fraction of the cells express CD73 and CD39 compared to the control (FIGS. 14C-14D). The lower values of ectonucleotidase expression in nonhematopoietic population are possibly associated with their underdetection due to debris in as-isolated samples. The primary cells isolated from bone chips for transcription levels was also examined and decreased expressions of CD73 (FIG. 14E) and CD39 (FIG. 14F) in OVX bone compared to corresponding healthy controls were found. Concomitant with the lower ectonucleotidase expressions, measurement of extracellular adenosine in the BM plasma showed a significant decrease in its concentration in OVX mice (FIG. 14G).

### ERs Regulate Ectonucleotidase Expression and Availability of Adenosine In Vitro

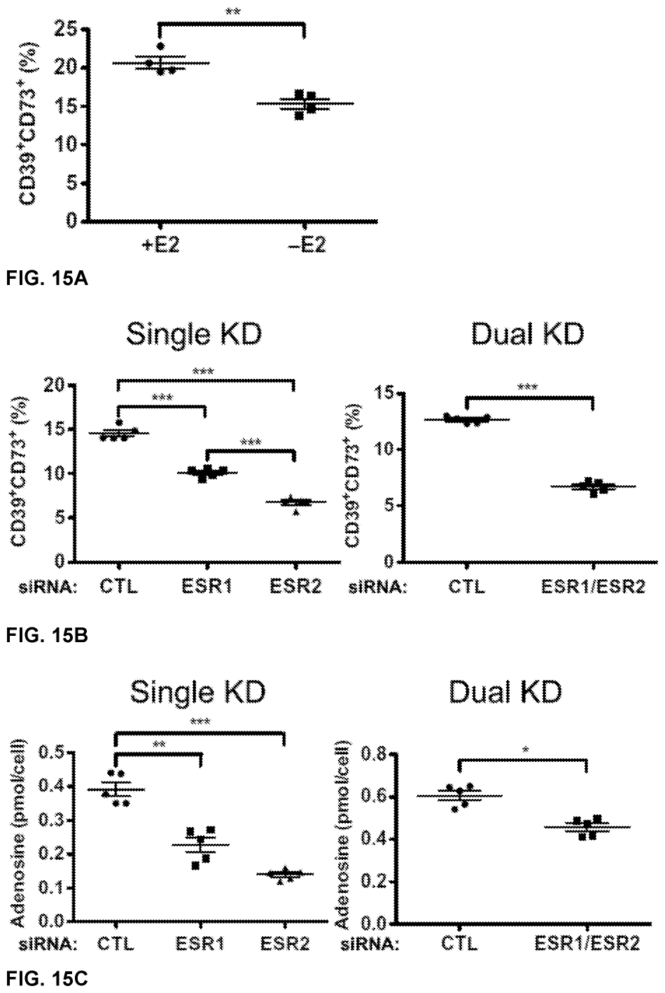

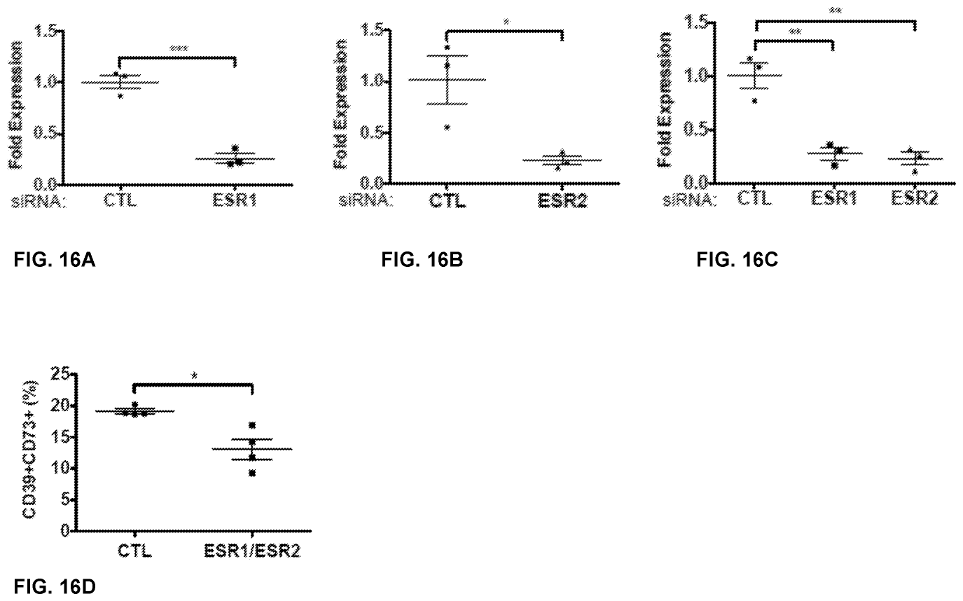

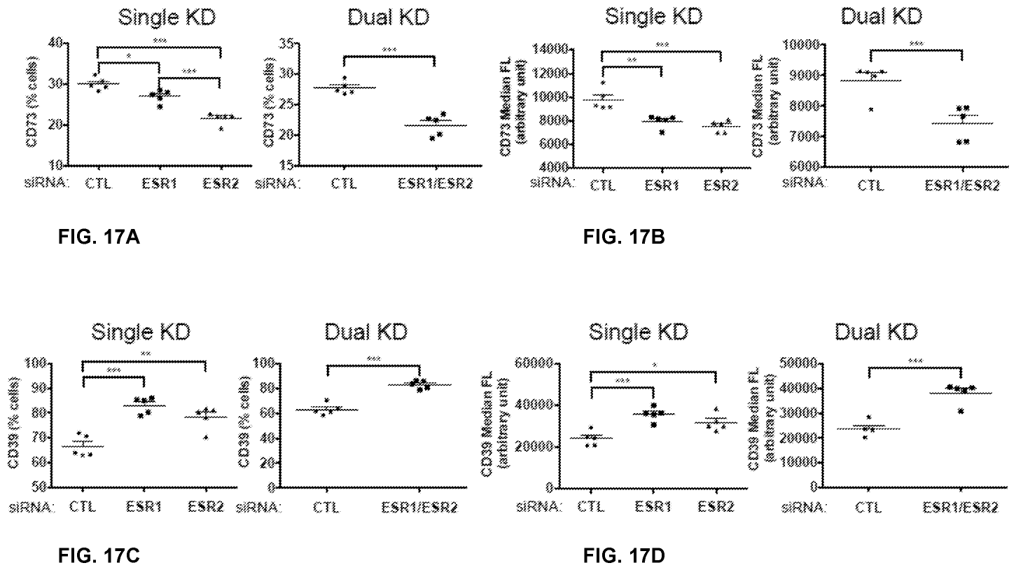

To explore whether estradiol (E2) is involved in maintaining CD73 and CD39 expression, E2 was withdrawn during culture as described above. Flow cytometric analyses of the osteoprogenitor cells revealed that the ratio of double-positive CD73- and CD39-expressing cells was decreased in the absence of E2 (FIG. 15A). Since ERs are the main receptors of E2, whether ERs regulate the expression of CD73 and CD39 and subsequently the derivation of extracellular adenosine in osteoprogenitor cells was further examined. Small interfering RNA (siRNA) oligonucleotides against Esr 1 and Esr2 were used. ER expression of osteoprogenitor cells was decreased in the knockdown of ESR1 (FIG. 16A), ESR2 (FIG. 16B), or dual knockdown of ESR1/ESR2 (FIG. 16C). Flow cytometric analyses of the osteoprogenitor cells revealed that the ratio of double-positive CD73- and CD39-expressing cells was decreased in dual knockdown groups (FIG. 15B). A similar trend in single ESR1 and ESR2 knockdown groups was also observed (FIG. 15B). Immunofluorescence staining of CD73 and CD39 in osteoprogenitor cells also demonstrated decreased double-positive cells in dual knockdown groups compared to control (FIG. 16D). Concomitant with the decrease in ectonucleotidase expressions, the concentration of extracellular adenosine decreased in all groups (FIG. 15C). The expression levels of individual ectonucleotidase (CD73 or CD39) was also examined. Flow cytometric analyses of CD73 alone showed a decrease in the percentage of CD73-expressing cells and median fluorescence in osteoprogenitors with both single and dual ER knockdown (FIGS. 17A-17B). Contrary to CD73, expression level of CD39 was found to increase in all groups (FIGS. 17C-17D).

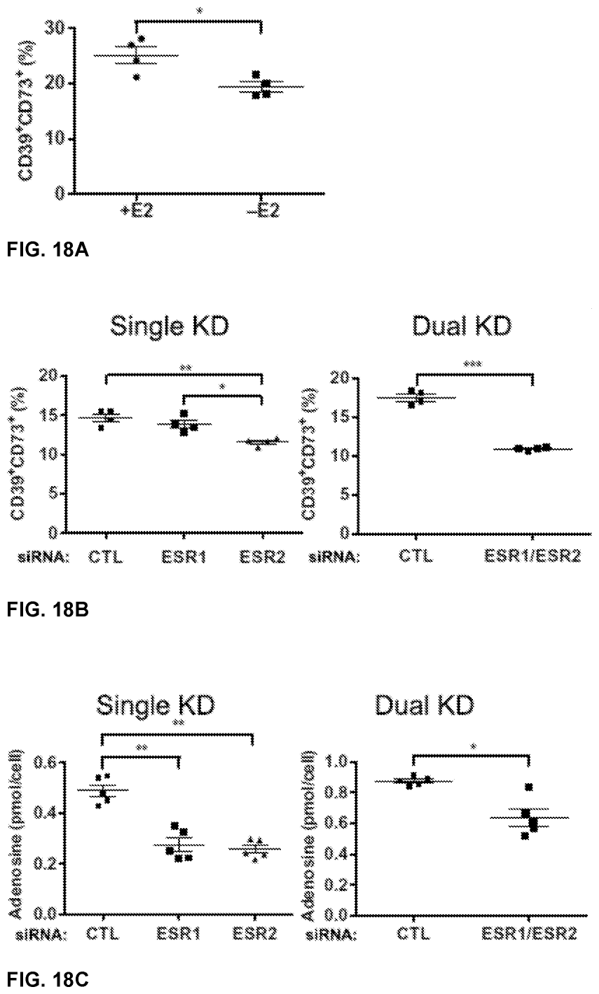

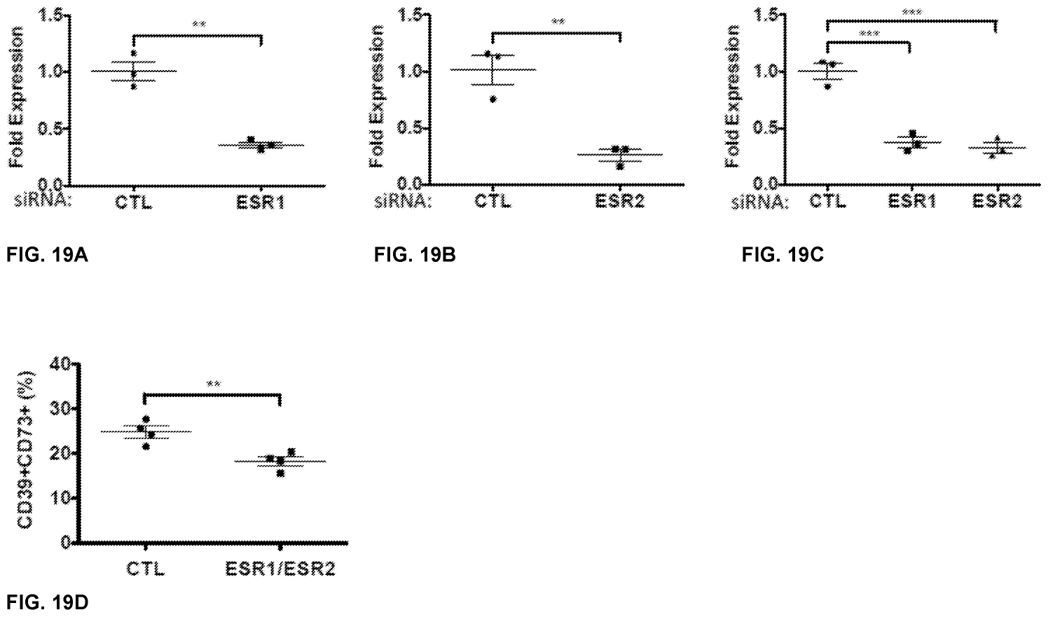

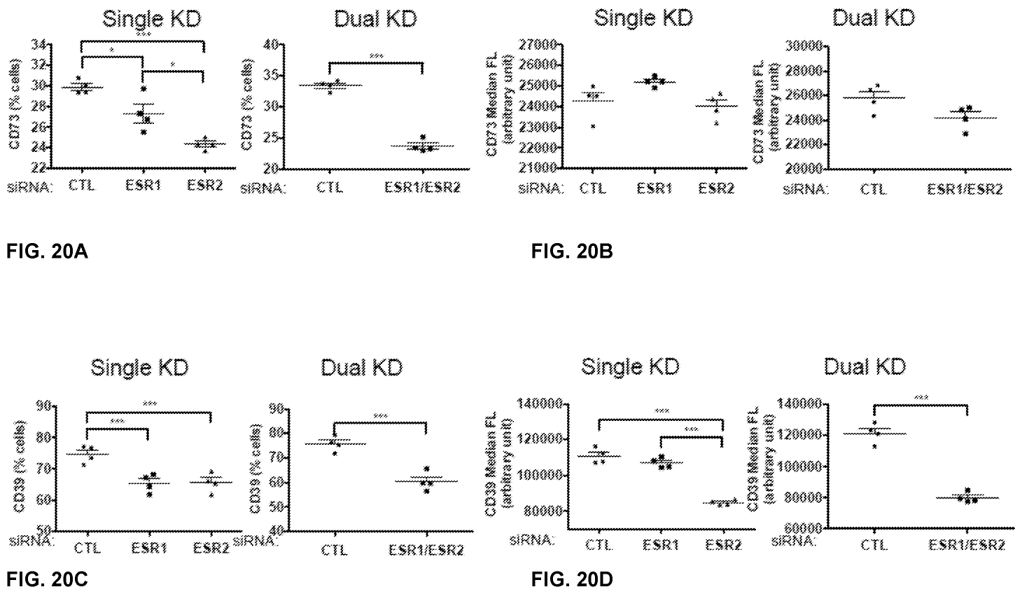

A similar analysis for mononuclear cells undergoing osteoclast differentiation was also carried out. Flow cytometric analyses of the osteoclasts revealed that the ratio of double-positive CD73- and CD39-expressing cells was decreased in the absence of E2 (FIG. 18A). ESR1 and ESR2 expressions of mononuclear cells undergoing differentiation were decreased in single knockdown of ESR1 (FIG. 19A), ESR2 (FIG. 19B), or dual knockdown (FIG. 19C). Flow cytometric analyses of CD73 and CD39 in osteoclasts revealed that the ratios of double-positive CD73- and CD39-expressing cells were decreased in ESR1 or ESR2 knockdown or dual knockdown (FIG. 18B). Immunofluorescence staining of CD73 and CD39 in osteoclasts also demonstrated decreased double-positive cells in dual knockdown groups compared to control (FIG. 19D). Extracellular adenosine levels showed a significant decrease in single and dual knockdown groups that correlated with the decreased ectonucleotidase expression (FIG. 18C). Flow cytometric analyses of CD73 alone showed a decrease in the percentage of cells expressing CD73 in single and dual knockdown groups (FIGS. 20A-20B). Analyses of CD39 showed a decrease in the percentage of cells expressing the marker in all the groups (FIGS. 20C-20D).

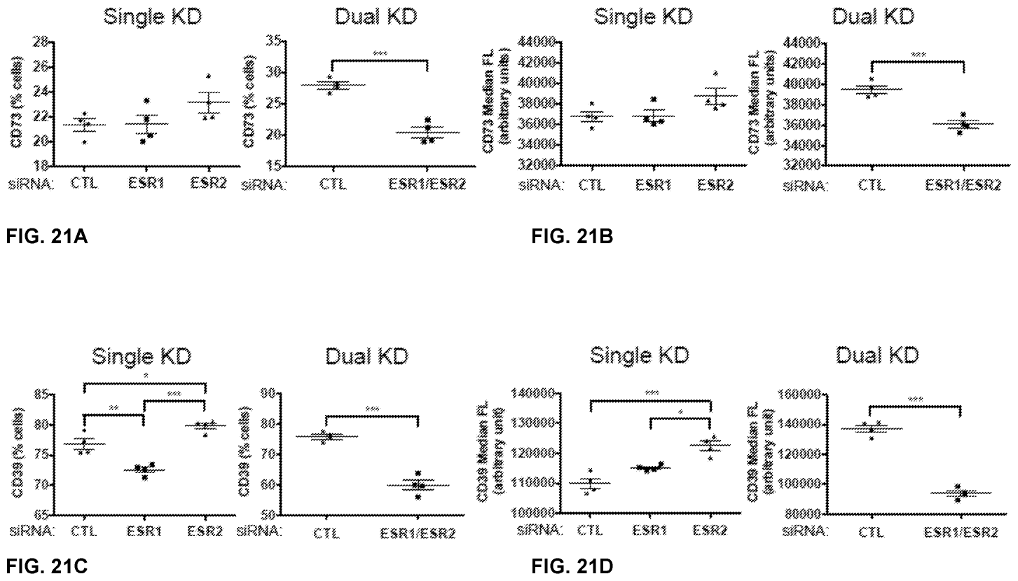

Since macrophages are precursors to osteoclasts, this cell population was also analyzed. Knockdown of ERs resulted in a decreased ratio of CD73-expressing cells in the dual, but not in single (ESR1 or ESR2), knockdown groups (FIG. 21A), along with a decreased median fluorescence of positive cells (FIG. 21B). Similarly, analysis of CD39 showed a significant decrease in CD39-expressing macrophages in ESR1 and dual knockdown groups but not in the ESR2 knockdown group (FIG. 21C). The median fluorescence of CD39-expressing cells decreased in dual knockdown but not in the case of single knockdowns (FIG. 21D).

### Adenosine Regulates Osteoblastogenesis and Osteoclastogenesis Through Adenosine A2BR In Vitro

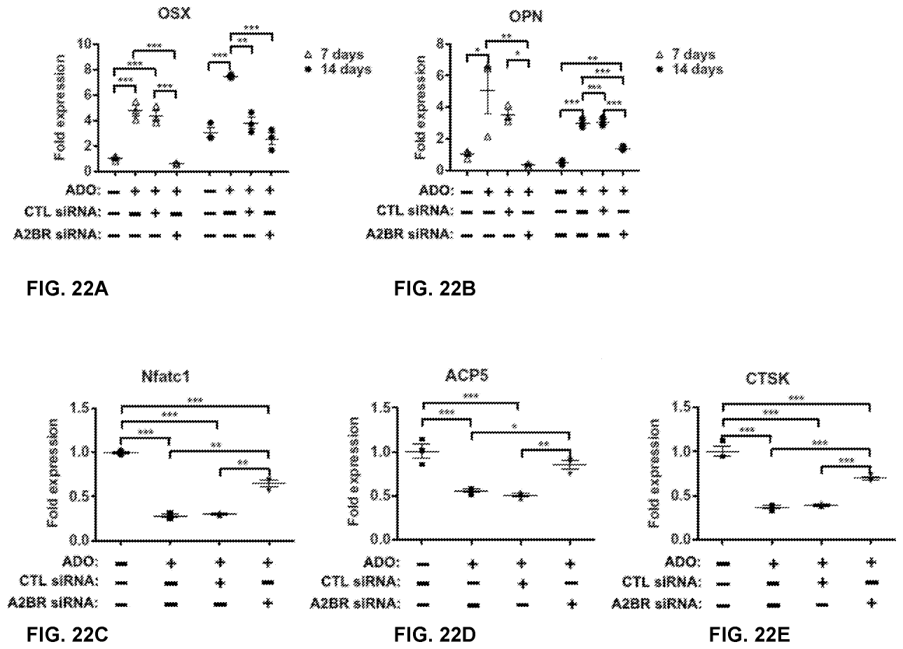

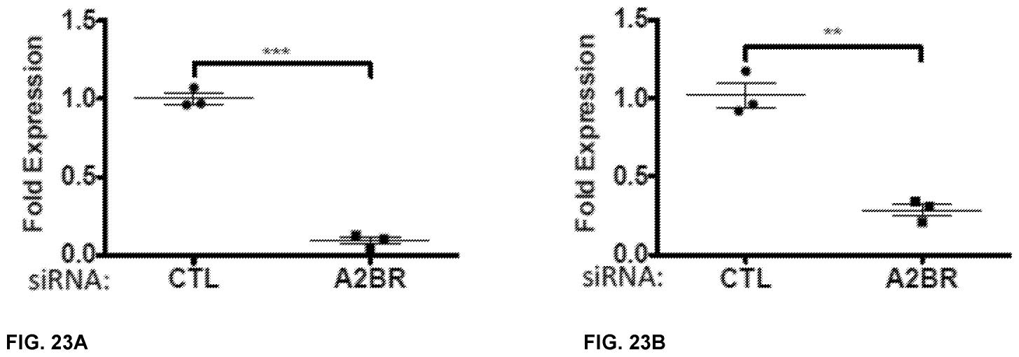

Mouse primary osteoprogenitor cells cultured in medium supplemented with adenosine devoid of osteogenic-inducing factors showed up-regulation of osteoblast-specific transcription factors osterix (OSX) and osteopontin (OPN) (FIGS. 22A-22B). To determine the involvement of A2BR signaling during adenosine-induced differentiation, osteoprogenitors and osteoclast precursors were treated with A2BR siRNA to perturb its expression (FIGS. 23A and 23B, respectively). Knockdown of A2BR expression abrogated the increased OSX and OPN expressions compared to groups with no siRNA and control (scrambled) siRNA (FIGS. 22A-22B). Contrary to osteoblastogenesis, extracellular adenosine diminished osteoclast differentiation as demonstrated by reduced expression of osteoclast transcription factor nuclear factor of activated T cells 1 (Nfatc1) and osteoclast-associated markers acid phosphatase type 5 (ACP5) and cathepsin K (CTSK) (FIGS. 22C-22E). This exogenous adenosine-mediated down-regulation of Nfatc1, ACP5, and CTSK gene expressions were reversed upon knockdown of A2BR expression (FIGS. 22C-22E). The diminished osteoclastogenesis was also verified by tartrate-resistant acid phosphatase (TRAP) staining, which showed an inhibitory effect of A2BR signaling during osteoclast differentiation. Together, the results suggest that A2BR activation promotes osteoblastogenesis and reduces osteoclastogenesis.

Treatment of Osteoporotic Mice with Adenosine A2BR Agonist Prevents Bone Loss

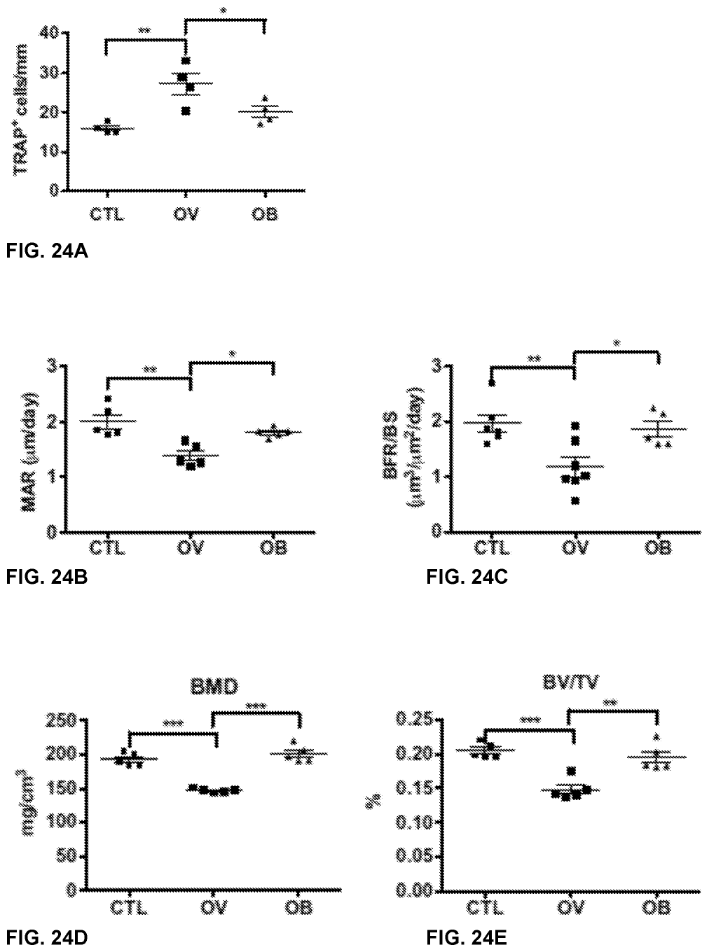

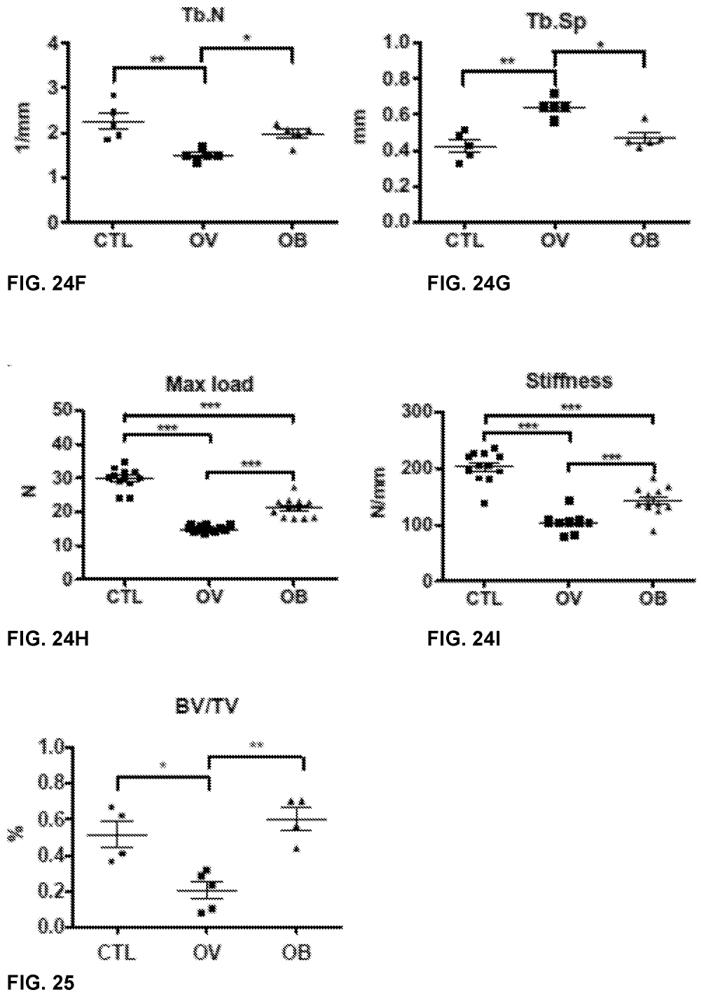

In vitro results demonstrating the dual action of A2BR signaling suggest that targeting this receptor could prevent bone loss. Immunohistochemical staining of A2BRs on the bone surface of OVX animal confirmed their presence on osteoporotic bone. OVX mice displaying bone loss were treated with the A2BR agonist BAY 60-6583 for 8 weeks and examined the changes in bone tissue. Hematoxylin and eosin (H&E) staining of the bone tissues displayed an attenuation of bone loss with BAY 60-6583 treatment. TRAP staining revealed less staining and a decreased number of TRAP-positive cells on the bone surface of mice treated with BAY 60-6583 compared to the vehicle control (FIG. 24A). Double-fluorescence bone labeling with calcein and alizarin complexone showed mineral deposition in all groups. Quantification of the images for mineral apposition rate (FIG. 24B) and bone formation rate (FIG. 24C) showed an abrogation of the bone loss in mice treated with BAY 60-6583. microCT analysis of vertebra demonstrated the decrease in bone mineral density (BMD), bone volume per total volume (BV/TV), and trabecular number (Tb.N), as well as an increase in trabecular spacing (Tb.Sp) in OVX animals. These changes were attenuated after treatment with BAY 60-6583 (FIGS. 24D-24G). A similar finding was also observed in femur again, showing that the bone loss was diminished upon treatment with BAY 60-6583 (FIG. 25). Mechanical measurements of the bone tissue further supported these findings. Cohorts treated with BAY 60-6583 showed improved maximum load and stiffness of bone tissues compared to the corresponding controls (FIGS. 24H and 24I, respectively). Although the treatment involving A2B agonist was not anticipated to increase the CD73/CD39 expression levels, immunofluorescence staining of CD73 and CD39 was carried out, and the results revealed no significant differences in their expression levels between the vehicle- and BAY 60-6583-treated groups.

### Discussion

This study establishes the pathophysiological correlation of altered ectonucleotidase CD39 and CD73 expressions and the extracellular adenosine availability in a postmenopausal osteoporotic model. The results demonstrated that OVX mice deficient in estrogen have lower expression of CD73 and CD39 in hematopoietic and nonhematopoietic cells of the BM that correlated with a decrease in extracellular adenosine. This is consistent with the in vitro studies where ER signaling maintained the coexpression of ectonucleotidases CD73 and CD39 and extracellular adenosine during the differentiation of osteoblasts and osteoclasts. However, results showed some differences in ER regulation of ectonucleotidases CD73 and CD39 between precursors undergoing osteoblastogenesis and osteoclastogenesis. Specifically, analyses following the ER perturbation showed that the percentage of cells expressing CD39 increased in osteoblasts and decreased in osteoclasts. Contrary to CD39, the ER perturbation showed that the percentage of cells expressing CD73 decreased in both osteoblasts and osteoclasts. These findings suggest that CD39 could have a disparate role between osteoblasts and osteoclasts and that CD73 has a dominant role in regulating the extracellular adenosine level over CD39 during ER signaling in osteogenic differentiation of progenitor cells. This agrees with prior findings that osteogenic cells from CD39 knockout mice exhibit diminished osteogenic differentiation only when extracellular adenosine uptake was restricted (He 2013). Furthermore, unlike CD73 knockout in male mice, CD39 knockout mice do not display an aberrant bone phenotype.

One of the cardinal reasons of osteoporosis is the compromised function of osteoblasts and excessive activity of osteoclasts. Studies have shown that A1 activation promotes osteoclast differentiation, while A2AR and A2BR exert inhibitory effects (He 2012; Mediero 2012; Mediero 2016; Mediero 2018;He 2013; He 2013; Mediero 2013). Consistent with these reports, the in vitro studies showed that the supplementation of adenosine promotes osteoblastogenesis while decreasing osteoclastogenesis. Furthermore, the results showed that this extracellular adenosine-mediated osteoblastogenesis and osteoclastogenesis involved A2BR signaling. The dual ability of the adenosine molecule to promote osteoblastogenesis while preventing excessive osteoclastogenesis through A2BR signaling could be an ideal therapeutic strategy to treat bone loss. As a proof of concept, administration of A2BR agonist BAY 60-6583 in OVX animals showed attenuation of bone loss. Despite being a partial agonist (Hinz 2014), administration of BAY 60-6583 showed therapeutic potential, suggesting that the adenosine A2BR may serve as a therapeutic target in postmenopausal osteoporotic disease. Here, stimulation of A2BR was used to compensate for the low levels of extracellular adenosine in the bone milieu. While the results show that A2B stimulation can be used to compensate for the low levels of extracellular adenosine in the bone milieu, the treatment does not introduce a feedback signaling to increase CD73/CD39 expression.

Unlike current treatments involving bisphosphonates that inhibit osteoclastogenesis, targeting A2BR signaling to modulate the function of osteoblasts and osteoclasts offers an unexplored therapeutic strategy for treating osteoporosis. However, the ubiquitous nature of adenosine receptors in the human body warrants a more targeted therapeutic approach to move such an approach to clinic. Adenosine is well known for its immunomodulatory effect and anti-inflammatory properties (Cronstein 1994; Vijayan 2017), implying that extracellular adenosine could also regulate the inflammatory-like environment present in osteoporosis. For example, proinflammatory cytokines have been implicated as primary mediators of accelerated bone loss during postmenopausal osteoporosis (Mundy 2007; Manolagas 2010; Weitzmann 2002). In addition to osteoclasts, multiple immune cells also participate in tissue resorption including destructive T cells and macrophages (Manolagas 2010; Cenci 2003; Weitzmann 2006). In line with adenosine as an immune suppressor, the lack of adenosine signaling in CD73 knockout mice develops spontaneous arthritis associated with inflammatory symptoms (Li 2014; Chrobak 2015).

Besides ectonucleotidases, other factors contributing to extracellular adenosine availability, including soluble CD73 (Yegutkin 2008), metabolism by adenosine deaminase (Sauer 2012), cellular transport by equilibrative nucleoside transporter 1 (Pastor-Anglada 2018), alternative CD38/CD203a/CD73, or CD203a/CD73 pathways (Horenstein 2013; Morandi 2015), were not investigated. Furthermore, OVX animals were used to study the pathophysiology of postmenopausal osteoporosis. Whether the same observations occur in osteoporosis during natural aging, secondary osteoporosis, human disease, and gender differences remain to be explored. Similar to females encountering postmenopausal osteoporosis, recent studies have shown that estrogen plays a crucial role in age-mediated male osteoporosis (Falahati-Nini 2000; Smith 1994).

In conclusion, a direct correlation between expression of the ectonucleotidase CD73 and CD39 and extracellular adenosine levels in a mouse model of postmenopausal bone loss was demonstrated. The active role of ER signaling in maintaining CD73 and CD39 expression and extracellular adenosine levels was established. As a proof of concept, it was shown that stimulation of A2BR using a small molecule can be used to compensate for low levels of extracellular adenosine and attenuate the associated bone loss. These results demonstrate that A2BR could be a therapeutic target for osteoporosis.

### Example 4

### In Vivo Sequestration of Innate Small Molecules to Promote Bone Healing

### Introduction

A leading concept in regenerative medicine is transplantation of tissue-specific cells, often supported by biomaterials, to promote tissue repair (Khademhosseini 2016). While this strategy has achieved some success, its broad clinical application is hindered by various challenges such as high costs, constraints associated with cell isolation and expansion, and limited in vivo engraftment of transplanted cells (Segers 2008; Salem 2010; Grayson 2015). Instead, mobilizing endogenous cells to augment the innate regenerative ability of tissues has been explored as an alternative (Dimmeler 2014; Chen 2011; Gonzalez 2018; Phinney 2017), and approaches that enable innate repair mechanisms hold great potential for tissue repair. Given that the function of endogenous cells is regulated by their microenvironment, potential of biomaterials and/or growth factors to create pro-healing niches for endogenous cells has been explored extensively (Chen 2011; Brusatin 2018; Webber 2016; Burdick 2016; Rosales 2016; Seale 2016; Lee 2010). Meanwhile, naturally-occurring small molecules are also appealing and equally powerful in regulating various cellular functions including tissue-specific differentiation of stem cells (Huangfu 2008; Borowiak 2009; Kang 2016). Although significant strides have been made in employing small molecules to direct cellular functions in vitro, harnessing small molecules towards tissue repair in vivo still remains limited.

In this study, whether biomaterial-assisted sequestration of small molecules could be used to augment endogenous cell function leading to improved tissue repair was determined. Adenosine is a small molecule ubiquitously present in the human body which acts as an extracellular signaling molecule through G-protein coupled adenosine receptors (Fredholm 2007; Haskó 2008). While the physiological concentration of extracellular adenosine is often insufficient to activate adenosine receptors (Lopez 2018), an increase in extracellular adenosine is observed following tissue injury, which is integral to the natural repair mechanism (Fredholm 2007; Carroll 2013; Ham 2012; Cronstein 2017). However, this increase is transient as adenosine is rapidly metabolized (Roszek 2019; Meling 2018). Although delivery of adenosine can be employed to activate adenosine signaling and address tissue dysfunctions, in practice, such an approach has remained elusive. This is mainly due to the ubiquitous nature of adenosine and the potential off-target effects associated with its systemic administration (Meling 2018; Biaggioni 1987; Kazemzadeh-Narbat 2015). Instead, approaches that localize adenosine signaling at the targeted tissue site can circumvent these limitations and open up new viable therapeutic strategies.

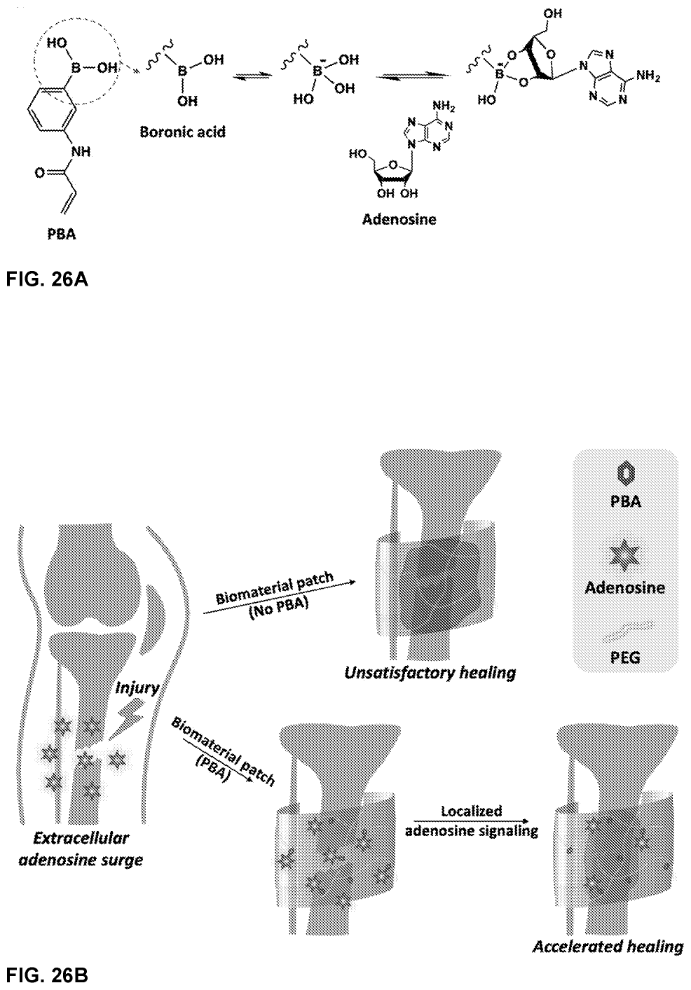

To this end, a biomaterial-based approach to sequester extracellular adenosine has been developed, capitalizing on its transient surge following trauma or to deliver exogenous adenosine to sustain the activation of adenosine signaling strictly at the injury site. Specifically, the ability of boronate molecule to bind to adenosine via dynamic covalent bonding was leveraged (Ryu 2018; Zhou 2018; Brooks 2016) (FIG. 26A). By employing a 3-(acrylamido)phenylboronic acid (PBA)-functionalized polyethylene glycol (PEG) network, it was demonstrated in vivo sequestration of adenosine and its application to accelerate bone repair in a murine model (FIG. 26B). Bone fracture was used as a model, due to its clinical relevance (Amin 2014; Haffner-Luntzer 2016; Burge 2007). Bone fracture is also well-suited for studying adenosine-mediated tissue repair, as extracellular adenosine and its receptors play a key role in maintaining bone homeostasis and function (Lopez 2018; Ham 2012; Mediero 2015) and have been proven to induce osteogenic differentiation of progenitor cells (Kang 2016; Shih 2014; Carroll 2012; Kang 2015).

It was demonstrated that implantation of the biomaterial patch following injury establishes an in-situ stockpile of adenosine, resulting in accelerated healing by promoting both osteoblastogenesis and angiogenesis. The adenosine content within the patch recedes to the physiological level as the tissue regenerates. In addition to sequestering endogenous adenosine, the biomaterial is also able to deliver exogenous adenosine to the site of injury, offering a versatile solution to utilize adenosine as a potential therapeutic for tissue repair.

### Materials and Methods

### Materials

Polyethylene glycol diacrylate (PEGDA) and N-acryloyl-6-aminocaproic acid (A6ACA) were synthesized as previously described (Hwang 2010; Ayala 2011). Briefly, 10 wt % of PEG (Mw-3.4 kDa, Sigma-Aldrich, Cat #P4338) solution was made by dissolving it in anhydrous dichloromethane (DCM) at room temperature with stirring in an argon environment. To this, 1.5 molar equivalents of triethylamine (Sigma-Aldrich, Cat #471283) and acryloyl chloride (Sigma-Aldrich, Cat #A24109) were added dropwise on ice. The reaction was continued overnight at room temperature followed by purification using Celite diatomaceous earth (Sigma-Aldrich, Cat #1026931000). The product was concentrated using a rotary evaporator and precipitated in ˜10-fold chilled diethyl ether. The resultant PEGDA was dried under vacuum overnight and purified by using a Sephadex fine G-25 column (GE Healthcare Life Sciences), followed by lyophilization. To synthesize A6ACA, 1 M of 6-aminocaproic acid (Sigma-Aldrich, Cat #A7824) was prepared by dissolving it in 1N sodium hydroxide, followed by reacting with 1.5 molar equivalent of acryloyl chloride added dropwise on ice. The reaction mixture was maintained at pH 8 for an hour and then gradually decreased to 3 by titrating with 5N hydrochloric acid. The product was extracted using ethyl acetate, dried over anhydrous sodium sulfate, and precipitated in chilled hexane. The resultant A6ACA was collected and dried overnight under vacuum. The successful completion of the reactions was confirmed by NMR as described previously (Ayala 2011).

### Macroporous Scaffold Fabrication

PEG macroporous scaffolds containing the PBA moieties were fabricated using a poly(methyl methacrylate) (PMMA) leaching method (Kang 2014). The polymer precursor solution was prepared by mixing PEGDA (10% w/v), 3-(acrylamido)phenylboronic acid (PBA, 1 M or 0.5 M; Sigma-Aldrich, Cat #771465), A6ACA (0.5 M) and Irgacure 2959 (0.5% w/v; Sigma-Aldrich, Cat #410896) in 80% ethanol. 20 μL of the precursor solution was added into a cylindrical polypropylene mold (5 mm in diameter) packed with 20 mg of PMMA microspheres (150-180 μm, Cospheric), followed by UV irradiation (365 nm) for 10 min. The resultant structures were soaked in acetone for 3 d to remove the PMMA beads with daily change of solvent followed by washing with deionized water. The macroporous scaffolds were trimmed to a uniform size of 6 mm in diameter and 2 mm in height. PEG macroporous scaffolds without PBA were also prepared in the same way. For sterilization, the scaffolds were soaked in 70% ethanol for 6 h, followed by washing in sterile PBS extensively for 3 d. The sterilized scaffolds were used for cell loading and in vivo experiments.

### Scanning Electron Microscopy (SEM)

Scaffolds were cut into thin slices and freeze-dried. To examine their porous structure, sliced scaffolds were sputter-coated with Au for 100 s (Denton Desk IV) and imaged by using a Philips XL30 ESEM under high vacuum mode.

### Nuclear Magnetic Resonance (NMR) Spectroscopy

To examine the extent of PBA incorporation, freshly prepared PBA scaffolds were thoroughly washed with deionized water to remove the unreacted PBA and freeze-dried. The samples were then minced and fully solvated in heavy water (D₂O) as described earlier (Ayala 2011). PBA₀ scaffolds were also prepared the same way. ¹H NMR spectra were recorded for all the samples using a 400 MHz Varian Inova spectrometer.

### Adenosine Loading and Release

Macroporous scaffolds were soaked in PBS supplemented with 6 mg/mL adenosine (Sigma-Aldrich, Cat #A4036) for 6 h and washed thoroughly to remove unbound adenosine. To measure the amount of sequestered adenosine, the scaffolds were soaked in acetate buffer (0.1 M, pH 4.5) for 2 h to completely release adenosine into the buffer, which was subsequently analyzed by using a UV/vis spectrophotometer (Beckman Coulter) at a wavelength of 260 nm. The concentration of the released adenosine was determined from a standard curve generated using adenosine solutions with known concentrations, ranging from 0.5 mM to 5 mM. To characterize the release profile of adenosine, the scaffolds were incubated in PBS with or without glucose (50 mM), or incubated in aMEM (Gibco, Cat #12561056) containing 10% (v/v) fetal bovine serum at 37° C. The concentration of adenosine in the buffer or medium was monitored as a function of time through UV/vis spectrophotometry.

### Cell Culture and Loading

Primary hMSCs were maintained and expanded in growth medium (GM) containing high-glucose DMEM (Gibco, Cat #11995065), 10% (v/v) fetal bovine serum (HyClone, Cat #SH3007103HI), 4 mM L-glutamine (Gibco, Cat #35050061), and 50 U/mL Penicillin/Streptomycin (Gibco, Cat #15140122). Cells were passaged at 70-80% confluency and used at Passage 5. Prior to cell loading, the sterilized scaffolds with and without adenosine were equilibrated in growth medium at 37° C. for 1 d. Cell loading was performed according to a published method (Kang 2018). Briefly, 20 μL of cell suspension containing 1 million hMSCs was loaded onto partially dehydrated scaffolds. The cell-laden scaffolds were kept in GM at 37° C. for 1 d to allow for cell infiltration. For in vivo study, these cell-laden scaffolds were then implanted subcutaneously in mice for 28 d. For in vitro study, the cell-laden scaffolds were cultured in GM supplemented with 3 mM phosphate at 37° C. and 5% CO₂ with medium change every other day. As a positive control for in vitro osteogenesis, a group of cell-laden scaffolds without adenosine were cultured in osteogenic-inducing medium (OM) made of GM supplemented with 10 mM β-glycerophosphate (Sigma, Cat #G9422), 50 μM ascorbic acid (Sigma, Cat #A4403), and 100 nM dexamethasone (Sigma, Cat #D2915) (Varghese 2010).

RNA Isolation and Quantitative Real-Time Polymerase Chain Reaction (qRT-PCR)

Osteogenic differentiation of the hMSCs as a function of time was evaluated by using qRT-PCR. The cell-laden scaffolds were lysed and homogenized in TRIzol Reagent (Invitrogen, Cat #15596018). Total RNA was extracted with chloroform and precipitated in isopropanol. 1 μg of each RNA sample was reverse transcribed with the iScript Reverse Transcription Supermix (Bio-Rad, Cat #1708841) following the manufacturer's instructions. The obtained cDNA was mixed with the forward and reverse primers of the target gene along with the iTaq SYBR Green Supermix (Bio-Rad, Cat #1725124) for qRT-PCR according to the manufacturer's protocol. The qRT-PCR was conducted in a Bio-Rad Thermal Cycler (CFX96) following the steps of an initial denaturation at 95° C. for 30 s for 1 cycle, amplifications at 95° C. for 5 s and 60° C. for 30 s for 40 cycles, and finally 95° C. for 10 min. The primer sequences are: osteocalcin (OCN; forward: TGAGAGCCCTCACACTCCTC (SEQ ID NO:19); reverse: ACCTTTGCTGGACTCTGCAC (SEQ ID NO:20)), osteopontin (OPN; forward: AATTGCAGTGATTTGCTTTTGC (SEQ ID NO:21); reverse: CAGAACTTCCAGAATCAGCCTGTT (SEQ ID NO:22)), osterix (OSX; forward: CATCTGCCTGGCTCCTTG (SEQ ID NO:23); reverse: CAGGGGACTGGAGCCATA (SEQ ID NO:24)), and 18s (forward: CCCTGTAATTGGAATGAGTCCACTT (SEQ ID NO:25); reverse: ACGCTATTGGAGCTGGAATTAC (SEQ ID NO:26)). The expression level of each target gene was calculated as ΔCt relative to the corresponding housekeeping gene (18s), converted to 2\hat{ }(−ΔΔCt) by normalizing to the group of PBA scaffolds cultured in growth medium for 7 days, and presented as fold change.

### Calcium Assay

To quantify the calcium deposition, cell-laden scaffolds were washed in deionized water, freeze-dried, lyophilized, and homogenized in 0.5 N HCl. The resultant homogenate was added into a Calcium Assay solution (Pointe Scientific, Cat #C7503) and the absorbance at 570 nm was recorded using a Multimode Detector (Shih 2014). The amount of free calcium ions (Ca²⁺) in the mixture was determined from a standard curve of calcium chloride solutions with known concentration and normalized to the dry weight of the corresponding scaffold.

### Subcutaneous Implantation and Tibial Fracture

All animal studies were conducted with the approval of the Institutional Animal Care and Use Committee (IACUC) at Duke University and complied with NIH guidelines for laboratory animal care. Female immunodeficient NOD.CB17-Prkdcscid/J mice (4-month-old, Jackson Lab) were used for subcutaneous implantation of hMSC-laden PBA scaffolds with and without adenosine. Female C57BL/6J mice (4-month-old, Jackson Lab) were used for all the experiments involving acellular scaffolds and patches. The mice were anesthetized with 2% isoflurane and administered with buprenorphine (1 mg/kg, sustained release, ZooPharm) through subcutaneous injection prior to surgical procedure. For subcutaneous implantation (Kang 2018), a roughly 1 cm-long incision was made on the back of each anesthetized mouse, and each cell-laden scaffold was implanted to the right side of the subcutaneous pouch. For tibial fracture (Baht 2017), the right tibia was first stabilized by inserting a 0.7 mm pin from the tibial plateau through the medullary cavity after removal of the skin proximal to the knee, a fracture was then created at the tibial midshaft with blunt scissors, and an 8 mm by 3 mm biomaterial patch was wrapped around the fracture site and held tight underneath the muscle. Upon completion, two drops of bupivacaine (0.5%, Hospira) were applied topically along the incision line, followed by closure with wound clips.

### Extracellular Adenosine Level in Bone Marrow

Bone marrow specimens from both the fractured limbs and the contralateral non-fractured limbs of the mice were collected at 30 min, 3 h, and 1 d following tibial fractures, respectively. Plasma was isolated from the bone marrow flush by centrifugation at 2,000 g, 4° C. for 20 min, and was subsequently diluted with an adenosine-protecting solution containing 0.2 mM dipyridamole, 5 μM erythro-9(2-hydroxy-3-nonyl)-adenine, 60 alpha,beta-methylene-adenosine 5′-diphosphate, and 4.2 mM ethylenediaminetetraacetic acid (EDTA). Extracellular adenosine content in the diluted plasma was quantified by using an Adenosine Assay Kit (Fluorometric; Abcam, Cat #ab211094) following the manufacturer's instructions. Briefly, each sample was mixed with a series of reagents including Adenosine Detector, Adenosine Convertor, Adenosine Developer and Adenosine Probe in an Adenosine Assay Buffer, and the mixture was incubated in dark for 15 min. Fluorescence intensity of the mixture was measured at 535 nm (excitation)/590 nm (emission) using a Multimode Detector, and the adenosine concentration was determined based on known adenosine standards.

### In Vivo Adenosine Sequestration

Freshly prepared PBA_{1.0} and PBA₀ scaffolds with identical dimensions were separately implanted into the subcutaneous pouches or the tibial fracture site of mice. For adenosine sequestration in the subcutaneous space, each mouse received a subcutaneous injection of 600 μL sterile saline or adenosine solution (0.25 mg/mL, 0.5 mg/mL) at ld after the subcutaneous implantation, and the scaffolds were excised 1 h later. For adenosine sequestration at the fracture sites, the scaffolds were excised at 3 d and 21 d after the implantation, respectively. The as-retrieved scaffolds were rinsed in PBS, minced, and soaked in acetate buffer (0.1 M, pH 4.5) for 2 h. The supernatant was subsequently collected, neutralized, and used for adenosine measurement.

### Microcomputed Tomography

Scaffolds retrieved from the subcutaneous implantation and fractured tibiae of mice were collected, processed in 4% paraformaldehyde at 4° C. for 3 d, and rinsed with PBS. The fixed samples and a phantom were loaded into a μCT scanner (vivaCT 80, Scanco Medical) and scanned at 55 keV with a pixel resolution of 10.4 μm. Reconstruction of the scanned images was performed using μCT Evaluation Program V6.6 (Scanco Medical), followed by generation of radiographs and 3D images using μCT Ray V4.0 (Scanco Medical). Bone mass was evaluated based on the reconstructed images and presented as a percentage of bone volume over total volume (% BV/TV). Bone mineral density (BMD) was determined by using the phantom with known hydroxyapatite content as a reference. Calluses of fractured tibiae were analyzed for % BV/TV based on 200 contiguous slices within 1 mm proximal and 1 mm distal of the fracture center according to a published study (Baht 2017).

### Histological Analyses

Fixed samples were decalcified in 14% EDTA (pH 8.0) at 4° C. for 5 d, rinsed in PBS, dehydrated and embedded in paraffin, and cut into 5 μm-thick sections by using a Leica rotary microtome. Prior to staining, each section was deparaffinized in CitriSolv (Decon Labs, Cat #1601) and rehydrated through graded alcohols and deionized water. For H&E staining, rehydrated sections were immersed in hematoxylin solution (Ricca Chemical, Cat #3536-16) for 4 min and then switched in eosin-Y solution (Ricca Chemical, Cat #2845-16) for 1 min. For osteocalcin (OCN) immunohistochemical analysis, rehydrated sections were immersed in a blocking buffer made of PBS, 3% bovine serum albumin and 0.1% Tween-20 for 1 h, and incubated with OCN primary antibody (1:100 in blocking buffer, rabbit polyclonal; Abcam, Cat #ab93876) overnight at 4° C. After rinsing thoroughly with PBS, the sections were treated with horseradish peroxidase (HRP)-conjugated secondary antibody (1:100 in blocking buffer, HRP-donkey anti-rabbit; Jackson ImmunoResearch, Cat #711-035-152) at room temperature for 1 h, followed by incubating in a developing solution containing 3-3′ diaminobenzidine (DAB) peroxidase substrate (Vector Laboratories, Cat #SK-4100) for 5 min to produce a brown reaction product. For Safranin-O staining of the fracture calluses, rehydrated tibia sections were immersed in 1% Safranin-O (Sigma, Cat #S8884) at room temperature for 1 h and counter-stained with 0.02% Fast Green (Sigma, Cat #F7258) and hematoxylin solution for 1 min. For tartrate-resistant acid phosphatase (TRAP) staining, rehydrated tibia sections were immersed in a 0.2 M sodium acetate buffer (pH 5.0) containing 50 mM tartaric acid (Sigma, Cat #228729), 0.5 mg/mL naphthol AS-MX phosphate (Sigma, Cat #N5000), and 1.1 mg/mL fast red TR (Sigma, Cat #F6760) for 1 h at 37° C. After rinsing in deionized water, the sections were counterstained in Mayer's hematoxylin solution (Sigma, Cat #MHS16) for 1 min. All the stained sections were subsequently dehydrated, covered with a mounting medium (Fisher Scientific, Cat #SP15-100), and imaged using a Keyence (BZ-X710) microscopy system.

### Immunofluorescence Imaging and Vessel Quantification

Rehydrated tibia sections were steam-treated in a citrate buffer (pH 6.0; Abcam, Cat #ab64236) for antigen retrieval and further immersed in blocking buffer for 1 h at room temperature. The sections were then incubated with endomucin primary antibody (1:100 in blocking buffer, rat polyclonal; Abcam, Cat #ab106100) overnight at 4° C., followed by addition of secondary antibody (1:200 in blocking buffer, Alexa Fluor 647-rabbit anti-rat; Abcam, Cat #ab169349) at room temperature for 1 h. All the sections were subsequently rinsed in PBS and mounted with an antifade medium containing DAPI (Invitrogen, Cat #P36971). Images were acquired using a Zeiss (Axio Imager Z2) microscopy system under the same exposure time for all groups. Quantification of blood vessel formation in calluses was conducted by using ImageJ (v1.52g) and represented as the percentage of EMCN-positive vessel area (red) to the callus area (blue) based on the immunofluorescence images. Ten images from each mouse tibia were used, and five mice from each group were included for the analysis.

### Statistical Analysis

The means with standard deviations (n≥3) are presented in the results. All the data were subjected to either two-tailed Student's t-test or one-way analysis of variance (ANOVA) with post hoc Tukey-Kramer test for multiple comparisons using GraphPad Prism 7. Any P-value of less than 0.05 was indicated with asterisk and considered statistically significant.

### Results and Discussion

Scaffolds Functionalized with PBA Groups Sequester Adenosine Both In Vitro and In Vivo

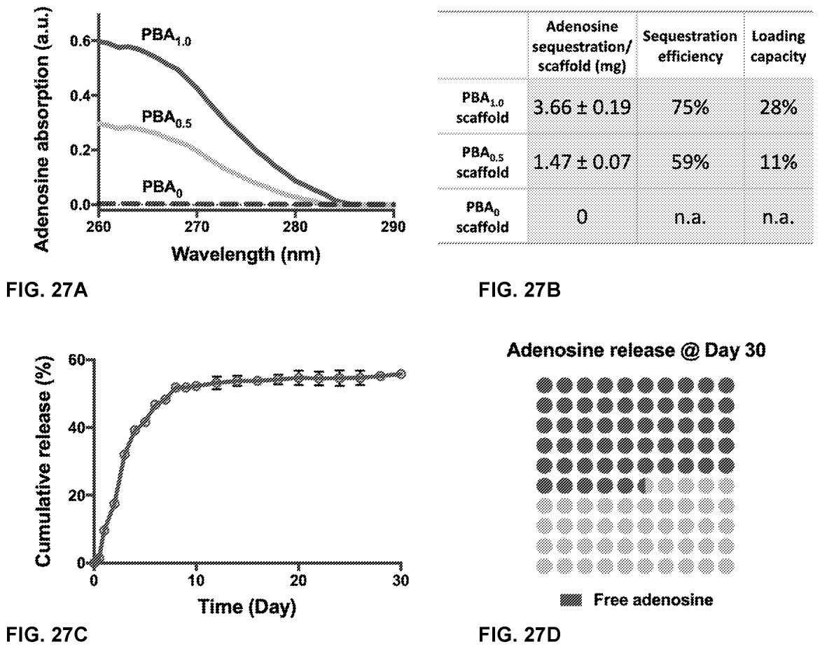

To examine the PBA-assisted sequestration and release of adenosine, macroporous PEG scaffolds were created containing varying amounts of PBA (0, 0.5 M, and 1 M as in the reaction mixture), termed as PBA₀, PBA_{0.5}, and PBA_{1.0}, respectively. The macroporous PEG scaffolds were developed by using polymethyl methacrylate (PMMA) microspheres as a porogen, resulting in an interconnected macroporous architecture (Kang 2014; Kang 2014). UV/vis analysis of the residual PBA in the reaction mixture and nuclear magnetic resonance (NMR) spectra of the resulting scaffolds suggest more than 90% of the PBA molecules were reacted and incorporated into the network. To determine PBA-mediated adenosine sequestration, the macroporous scaffolds with different levels of PBA were incubated in an excess adenosine solution (6 mg/mL in PBS) for 6 h and the bound adenosine was measured using UV/vis spectroscopy. As shown in FIG. 27A, the amount of sequestered adenosine increased as the amount of PBA within the scaffold increased. Specifically, the PBA_{1.0} scaffolds had a sequestration efficiency (the amount of PBA moieties involved in adenosine binding) of 75% with a loading capacity (weight percentage of adenosine in the scaffold) of 28%, while those of the PBA_{0.5} scaffolds were 59% and 11%, respectively (FIG. 27B). On the contrary, the PEG scaffolds without PBA moieties (i.e. PBA₀) had no detectable adenosine content, suggesting that the loading of adenosine was primarily due to the PBA moieties. Since the PBA_{1.0} scaffolds sequestered more adenosine compared to PBA_{0.5}, they were used for the rest of the studies. The release of adenosine was tested by incubating the PBA_{1.0} scaffolds in a cell culture medium depleted of nucleosides, which showed a robust release during the first 10 d followed by a plateau (FIGS. 27C-27D).

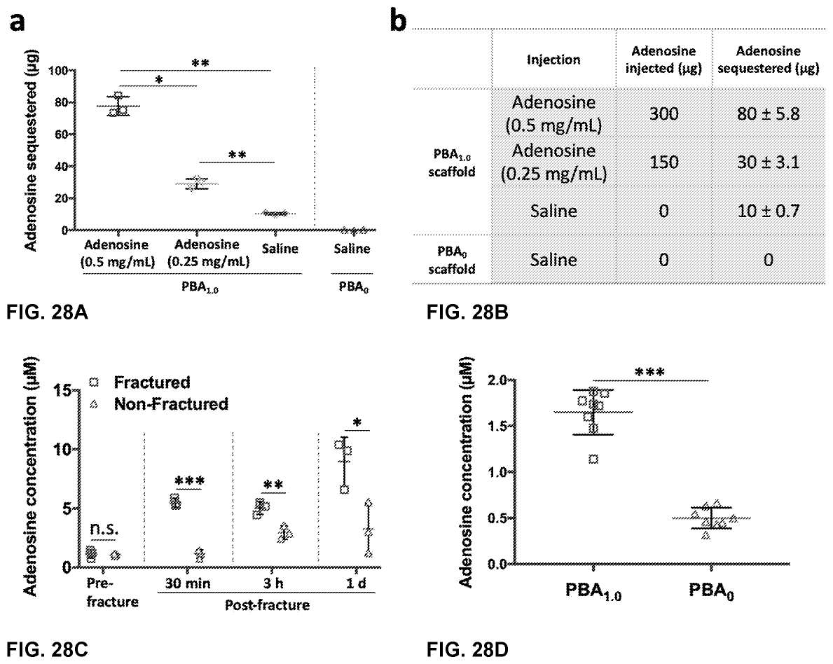

Having established the ability of PBA scaffolds to sequester and release adenosine in vitro, their potential to sequester adenosine in vivo was next examined. The ability of PBA_{1.0} scaffolds to sequester adenosine in vivo was first assessed by using a subcutaneous model. Roughly, 600 μL of sterile saline solution containing varying amounts of adenosine (0, 0.25 or 0.5 mg/mL) was injected into an area adjacent to the scaffolds, which had been implanted subcutaneously into mice for 1 d. The scaffolds were retrieved within 1 h and analyzed for the sequestered adenosine. As anticipated, the PBA_{1.0} scaffolds retrieved from the cohort injected with 0.5 mg/mL adenosine had higher adenosine content compared to that received 0.25 mg/mL adenosine or saline alone (FIGS. 28A-28B). The PBA_{1.0} scaffolds from the cohort that received only the saline injection were also positive for adenosine, albeit a small amount, which is attributed to the endogenous adenosine present at the site of implantation. On the contrary, no adenosine was detected in the PBA₀ scaffolds, further corroborating the necessity of PBA moieties for adenosine sequestration.

Although the physiological extracellular adenosine concentration in most organs is low, its level in the extracellular milieu is known to increase following trauma or injury (Carroll 2013; Ham 2012). Consistent with the existing knowledge, the time-dependent analyses of extracellular adenosine following unilateral tibial fracture of mouse showed a significant increase in the adenosine level at the injury site compared to that at the non-fractured contralateral site (FIG. 28C). A roughly 10-fold increase in extracellular adenosine was observed within 1 d following the injury. To determine the ability of PBA scaffolds to sequester extracellular adenosine by leveraging its surge following fracture, scaffolds were implanted at tibial fracture site upon injury and excised after 3 d. Compared to the PBA₀ scaffolds retrieved from the fracture site, those as-retrieved PBA_{1.0} scaffolds contained a significantly higher amount of adenosine (FIG. 28D). This increased adenosine content within the implant was found to be diminished to a concentration similar to pre-fracture levels by 21 d. Together, the results suggest that biomaterials containing PBA molecules can be used to sequester and enrich extracellular adenosine locally in response to injury.

### PBA-Adenosine Conjugation Promotes Stem Cell Osteogenesis Both In Vitro and In Vivo

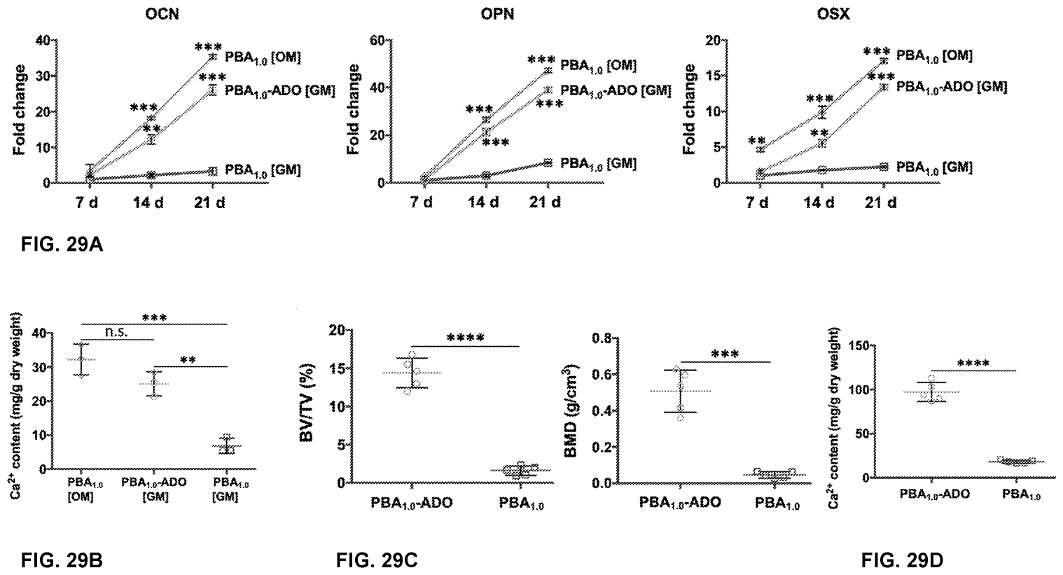

The osteoanabolic potential of adenosine bound to the scaffold was examined in vitro in a 3D culture by using human mesenchymal stem cells (hMSCs) as a cell source. Towards this, macroporous scaffolds with and without adenosine (PBA_{1.0}-ADO and PBA_{1.0}, respectively) were loaded with hMSCs and cultured in growth medium (GM). Cell-laden PBA_{1.0} scaffolds cultured in osteogenic-inducing medium (OM) were used as a positive control. It has been previously shown that macroporous scaffolds with an interconnected macroporous structure can facilitate infiltration of the loaded cells, allowing their homogenous distribution within the scaffold (Kang 2014; Kang 2014). PicoGreen DNA assay as a function of culture time showed comparable levels of DNA content in all groups. Osteogenic differentiation of hMSCs in various culture conditions was evaluated through time-resolved quantitative analyses for multiple osteogenic genes—osteocalcin (OCN), osteopontin (OPN) and osterix (OSX). As shown in FIG. 29A, the expressions of OCN, OPN, and OSX were consistently up-regulated throughout 21 d of culture in the PBA_{1.0}-ADO scaffolds similar to the positive control. In contrast, the expressions of osteogenic markers remained low in corresponding cultures with scaffolds lacking adenosine. Consistent with these findings, quantification of calcium content exhibited significantly higher calcium deposition in the PBA_{1.0}-ADO scaffolds compared to the PBA_{1.0} scaffolds with the same culture condition at the end of 21 d (FIG. 29B). These results suggest that the sequestered adenosine within the scaffolds promoted osteogenic differentiation of hMSCs akin to cultures involving medium supplemented with adenosine (Kang 2016; Shih 2014).

The potential of adenosine-bound scaffolds to support in vivo bone formation by adopting an ectopic model was next evaluated (Kang 2018). Both PBA_{1.0}-ADO and PBA_{1.0} scaffolds loaded with hMSCs were implanted into the subcutaneous space of immunodeficient mice for 28 d. Upon retrieval, the PBA_{1.0}-ADO scaffolds were found to be opaque. Radiographs generated from the microcomputed tomography (μCT) scans showed a strong optical signal from the PBA_{1.0}-ADO scaffolds, which is consistent with the gross appearance, suggesting in vivo calcification and the presence of hard tissue formation. The 3D rendering of the excised PBA_{1.0}-ADO scaffolds showed an even distribution of mineral deposition within the scaffolds, as indicated by both top and oblique views. Conversely, the excised PBA_{1.0} scaffolds did not display this opaque appearance nor apparent calcification. Based on the quantification of μCT results, the PBA_{1.0}-ADO scaffolds had a bone volume ratio (BV/TV) of 14.4% and a bone mineral density (BMD) of 0.51 g/cm³, compared to 1.6% and 0.05 g/cm³ found within the PBA_{1.0} group (FIG. 29C). Measurement of calcium content within the scaffolds, 97.3±4.8 mg/g dry weight in the PBA_{1.0}-ADO and 18.2±0.8 mg/g dry weight in the PBA_{1.0} scaffolds (FIG. 29D), further confirmed higher in vivo calcification of the cell-laden PBA_{1.0}-ADO scaffolds.

Bone tissue formation was further evaluated by histological characterization. Hematoxylin and eosin (H&E) staining of the excised implants showed dense extracellular matrix (ECM), resembling that of the bone tissue, in the cell-laden PBA_{1.0}-ADO scaffolds, whereas the corresponding PBA_{1.0} group had minimal bone tissue formation. Furthermore, positive staining of OCN, an ECM protein secreted by osteoblasts, was seen throughout the PBA_{1.0}-ADO scaffolds. Together, the findings suggest that the adenosine-loaded scaffolds supported osteogenic differentiation of the transplanted hMSCs and promoted ectopic bone formation, which further corroborates the osteoblastogenic function of adenosine.

### PBA-Mediated Adenosine Sequestration Promotes Bone Fracture Healing

A tibial fracture model was employed to investigate the role of biomaterial-assisted sequestration of adenosine in bone repair, which is a comprehensive process involving cartilaginous callus formation at the injury site, endochondral ossification within the callus, and callus/bone remodeling (Einhorn 2015). Stabilized fractures were induced unilaterally at tibial midshafts in mice (Baht 2017), and biomaterials with uniform dimensions were used to cover the fracture sites. In addition to PBA₀ and PBA_{1.0} patches, patches pre-loaded with exogenous adenosine (PBA_{1.0}-ADO) were also used. The fracture healing as a function of time was monitored using radiographic and histomorphometric analyses.

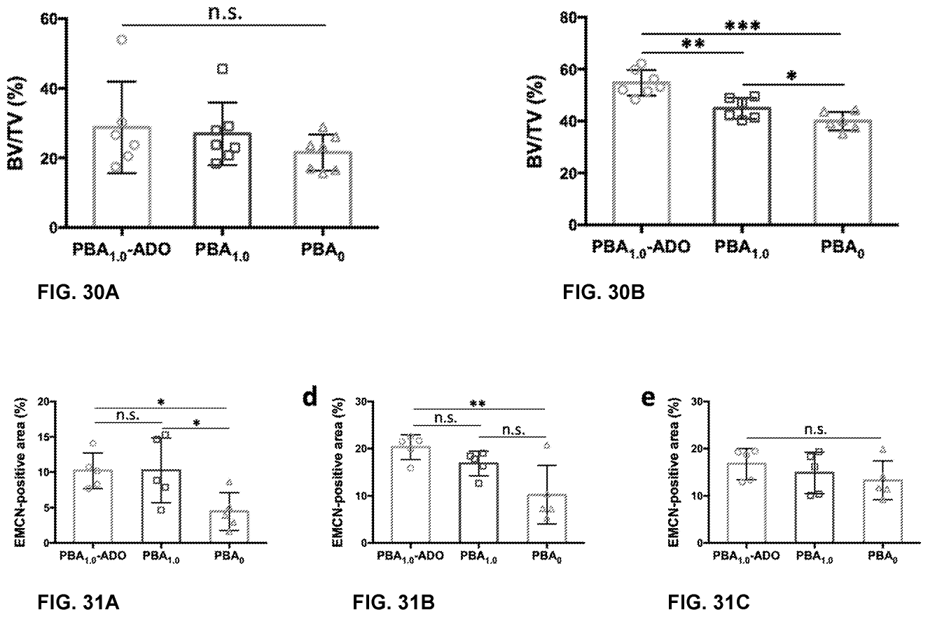

At 7 d, the fractures were still evident in all groups owing to the minimal mineralization of the calluses. As time progressed, the calluses calcified, and the extent of mineralization was found to correlate with the type of intervention, where both the PBA_{1.0}-ADO and the PBA_{1.0} groups exhibited a better bridging of the fracture. By 21 d, growing calluses eventually bridged the fracture gaps in all groups. Interestingly, radiographic images at 21 d showed cortical bridging only in groups treated with PBA_{1.0}-ADO and the PBA_{1.0}, suggesting a faster healing compared to those treated with PBA₀ (Einhorn 2015). Concomitant with these observations, analysis of the fracture sites at 21 d from axial view revealed better remodeled patterns and more organized lamellar bone formation in cohorts that received either PBA_{1.0}-ADO or PBA_{1.0} patches. These findings were further confirmed by the quantification of the μCT scans at 14 d (FIG. 30A) and 21 d (FIG. 30B). By 14 d, bone volume was higher in both the PBA_{1.0}-ADO and the PBA_{1.0} groups, albeit with no statistical significance. The differences in bone formation were apparent by 21 d, where the fractures treated with PBA_{1.0}-ADO and PBA_{1.0} exhibited significantly higher bone volume ratio within the calluses compared to those treated with PBA₀. Together, the results demonstrate the prevalent role of localized adenosine signaling in promoting callus maturation and fracture healing. When the biomaterial patch was dosed once with exogenous adenosine, as in the case of the PBA_{1.0}-ADO group, the healing was further improved, mostly due to the higher amount of adenosine available.

Given the importance of the evolution of cartilaginous tissue, vascularization, and osteoclast-driven bone resorption in fracture healing (Einhorn 2015), the effect of biomaterial-mediated adenosine signaling on cartilaginous tissue, blood vessel formation, and osteoclast activity during healing was also examined. Intense cartilage formation within the calluses of both the PBA_{1.0}-ADO and the PBA_{1.0} groups at 7 d was observed, followed by cartilage resorption over time suggesting endochondral ossification. In contrast, the animals treated with the PBA₀ showed delayed cartilaginous tissue formation and remodeling. Cartilaginous tissues still remained in the calluses of these animals at 21 d. Concurrent with these findings, an intervention-specific change in vascularization of the calluses was also observed (FIGS. 31A-31C). Specifically, more endomucin (EMCN)-positive blood vessels were detected in both the PBA_{1.0}-ADO and the PBA_{1.0} groups compared to the PBA₀ cohort at 7 d (FIG. 31A) and 14 d (FIG. 31B). The improved callus vascularization in the presence of PBA-mediated adenosine signaling may be directly linked to the established role of adenosine in promoting angiogenesis (Antonioli 2013; Montesinos 2002). The improved osteoblastogenesis observed in these groups could also contribute to the increased vascularization (Kusumbe 2014; Xu 2018). While there were differences in vascularization among the different groups at early time points, no intervention-dependent differences in blood vessel content were observed at 21 d (FIG. 31C). Analyses of osteoclast activity via TRAP staining showed increased TRAP-positive area with time in all the groups. Particularly, higher percentage of TRAP-positive area in the PBA_{1.0}-ADO cohort at 21 d, indicating higher bone remodeling, which could be associated with high levels of bone formation.

To summarize, the cohorts treated with PBA_{1.0} or PBA_{1.0}-ADO showed a better healing outcome compared to those treated with PBA₀, as evident by the extent of callus maturation, endochondral calcification, and angiogenesis suggesting that biomaterial-mediated localization of adenosine signaling promote bone healing. Furthermore, the increased adenosine concentration from the biomaterial-mediated sequestration recedes to the physiological level with healing, which underscore the translational potential of the described strategy. While PBA-mediated sequestration of endogenous adenosine alone promoted fracture healing, the augmentation of adenosine level with a one-time supplement of exogenous adenosine (i.e. PBA_{1.0}-ADO) further improved callus vascularization and healing outcome. Note that the PBA_{1.0} sequestered only a small fraction of the adenosine being released by cells (FIG. 28D); further improvement of biomaterial design, such as increasing PBA content or changing the architecture to increase surface-to-volume ratio, could be used to increase the sequestration efficiency. Such an approach will imbibe more adenosine from the milieu following injury and sustain its local concentration and could eliminate the need for exogenous adenosine entirely. Nonetheless, the results presented in this study showed the potential of using a PBA-containing biomaterial to boost the adenosine concentration at the fracture site and leverage the natural repair mechanism involving adenosine signaling to promote fracture healing.

### Conclusions

This study demonstrates that sequestration of adenosine, a native small molecule, by biomaterials at the fracture site can be used to promote bone fracture healing. The sequestration and release of adenosine was achieved by harnessing the ability of boronate molecules to form dynamic covalent bonds with cis-diol molecules such as adenosine. This biomaterial approach sustained an elevated concentration of adenosine locally by leveraging the surge of extracellular adenosine following injury and created a pro-regenerative milieu through localized adenosine signaling, resulting in improved bone repair. Besides sequestering endogenous adenosine, the biomaterial can also be used to deliver exogenous adenosine to the injury site, especially in pathological situations encountering diminished extracellular adenosine. By enabling a prolonged adenosine signaling, this biomaterial approach circumvents potential off-target effects associated with the systemic administration of adenosine, which is a major hurdle in harnessing adenosine signaling as a potential therapeutic.

The biomaterial-assisted sequestration of extracellular adenosine can be used to create an in-situ stockpile of the small molecule, which can be conveniently replenished non-invasively through injections. For example, local modulation of the adenosine signaling may be used to prevent repeated fractures, which are commonly observed in the aged population, as well as in patients suffering with osteoporosis and other bone-degenerating diseases. The biomaterial can be adapted accordingly to mirror the innate repair mechanism by modulating the extent of adenosine sequestration.

### Example 5

### Microgels with pH Sensitive Delivery of Adenosine

### Synthesis of ADO-Ketal Bone Targeting Nanocarrier

ADO containing bone targeting nanocarrier was prepared in two 3 steps. Firstly, a photopolymerizable polymer with bone targeting moiety was synthesized from hyaluronic acid (HA) via the introduction of methacrylate (MA) group followed by the bone targeting moiety alendronate (Aln). Secondly, adenosine (ADO) was conjugated with 2-(methacryloyloxy)ethyl acetoacetate (2MAEA) via ketal bond between the vicinal diol groups of ADO and the ketone group of 2MAEA to obtain 2MAEA-ADO. Finally, the nanocarrier was synthesized by copolymerizing the polymer (HA-MA-Aln) and the 2MAEA-ADO in an inverse emulsion suspension polymerization method.

### Synthesis of Hyaluronic Acid Methacrylate (HA-MA)

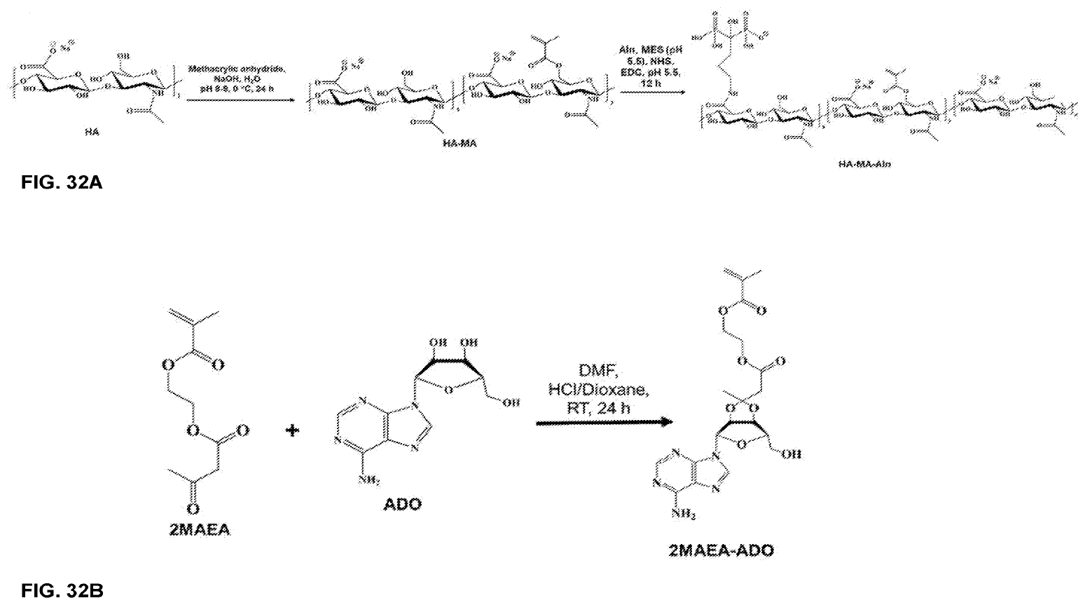

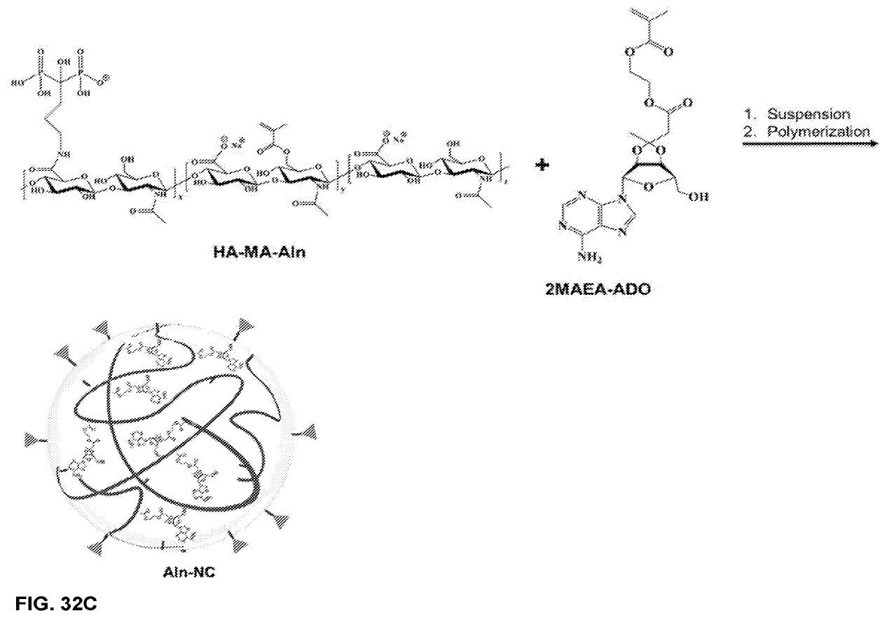

Photopolymerizable methacrylate group was introduced into HA via esterification of the hydroxyl group upon reacting HA with methacrylic anhydride (FIG. 32A). Briefly, HA was dissolved in deionized (DI) water. Methacrylic anhydride (20 equivalent) was added to the HA solution and the pH of the reaction mixture was adjusted to 8-8.5 by adding 5 N NaOH. The reaction was continued for about 24 h at 4° C. Excess of ice-cold ethanol-acetone mixture (1:1) was added to precipitate the product. The precipitate was filtered, washed several times with ice-cold ethanol-acetone mixture. Next, the polymer was dissolved in DI water and dialyzed for 4 days (using 3.5 kDa membrane) against DI water. The solution was freeze dried to obtain the methacrylated HA. The polymer was characterized by using a combination of FTIR and ¹HNMR spectroscopy. FTIR spectra of the modified HA showed the presence of peaks corresponding to ester C═O and methacrylate C═C stretching frequencies at 1720 cm⁻¹ and 1610 cm⁻¹ respectively, confirming successful methacrylation. The degree of methacrylation, determined via ¹HNMR spectroscopy, was found to be 32±2% per dimeric repeating unit.

### Synthesis of Alendronate-Conjugated HA-MA (HA-MA-Aln).

HA-MA was modified with the bone targeting agent alendronate (Aln) via amide coupling reaction between the carboxylic acid group of HA-MA and the amine group of Aln (FIG. 32B). Briefly, HA-MA was dissolved in MES buffer of pH 5.5 to yield a concentration of 10 mg/mL. EDC (1.0 equivalent) and NHS (1.0 equivalent) were gradually added to HA-MA solution at 15 min intervals. After 30 min, Aln (0.25 equivalent) was added to the reaction mixture. The reaction was continued for about 12 h at room temperature. The mixture was then dialyzed by using a 3.5 kDa membrane against DI water for 4 days and the resulting purified solution was lyophilized to obtain alendronate conjugated HA-MA (HA-MA-Aln). The polymer was characterized by using FTIR and ¹HNMR spectroscopy. The degree of Aln conjugation, determined via ¹HNMR spectroscopy, was found to be ˜18±2% with respect to the dimeric repeating unit of HA.

### Synthesis of ADO-ketal 2MAEA-ADO

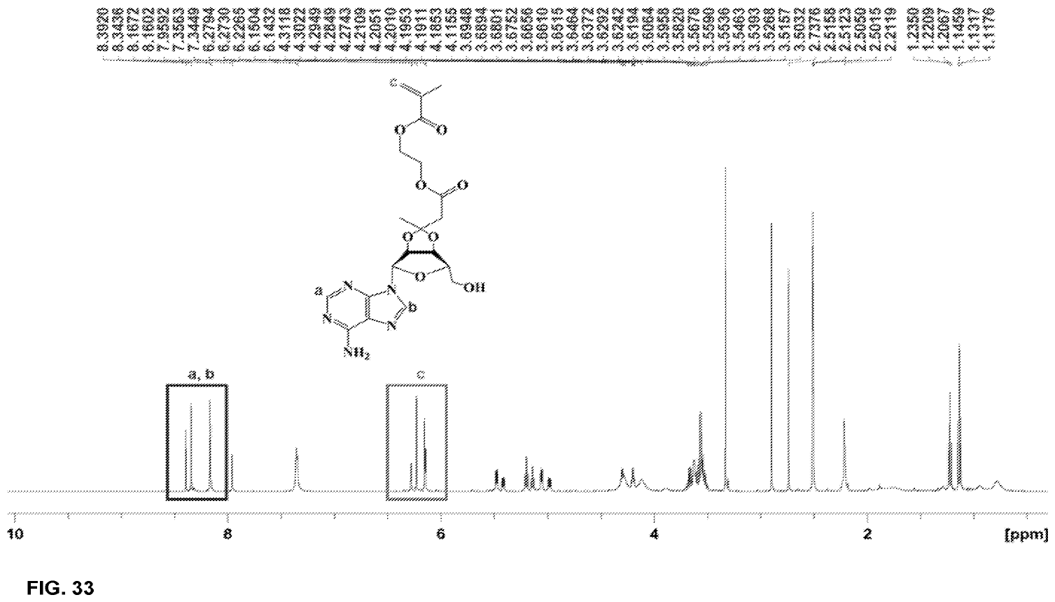

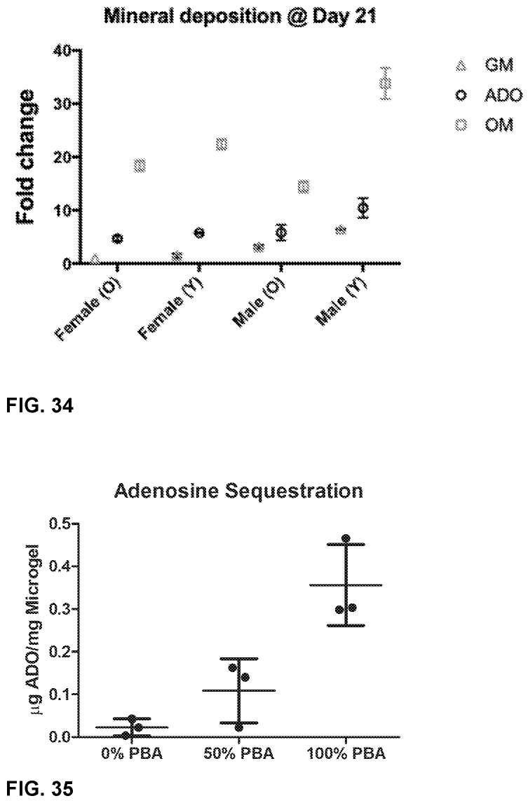

ADO was conjugated via ketal bond formation between the vicinal diol groups of ADO and the ketone group of 2MAEA (FIG. 32A). Briefly, 2-(Methacryloyloxy)ethyl acetoacetate (2MAEA) (2 equivalent), adenosine (1 equivalent) and triethyl orthoformate (2 equivalent) were dissolved in 18 ml of DMF. Then the 4 M HCl in 1,4-dioxane (2 equivalent) was added to the mixture. The reaction mixture was stirred under room temperature for 24 h. The reaction mixture was partitioned between dichloromethane (DCM, 75 mL) and a saturated aqueous sodium bicarbonate solution (25 mL). The aqueous phase was further washed with DCM (2×25 mL), the organic layers were combined and concentrated on a rotary evaporator. The product was then precipitated by addition of dry diethyl ether, filtered, and dried in high vacuum at 40° C. overnight. The successful conjugation of ADO to 2MAEA was confirmed via ¹HNMR spectroscopy as the product showed peaks at 8.1-8.3 ppm corresponding to adenosine aromatic protons in addition to peaks corresponding to acrylate protons of 2MAEA at 6.1-6.4 ppm (FIG. 33).

### Nanocarrier Synthesis and Purification

The nanocarrier was prepared via inverse emulsion photopolymerization method. Briefly, HA-MA-Aln was dissolved in DI water (50 mg/mL). 2MAEA-ADO was dissolved in dimethyl sulfoxide (50 mg/mL). A photoinitiator lithium phenyl-2,4,6-trimethylbenzoylphosphinate (LAP) was also separately dissolved in DI water (50 mg/mL). 200 μL of 2MAEA-ADO solution, 80 μL of HA-MA-Aln and 20 μL of LAP were then mixed together (2MAEA-ADO:HA-MA-Aln:LAP=1:0.4:0.1). The final solution was then emulsified in a continuous phase consisting of cyclohexane (10 mL) containing 2.5% w/v Span 80 surfactant through ultrasonication for 60 sec. The nanodroplets were crosslinked via UV irradiation for 10 min under constant stirring at 300 rpm. The photo-crosslinked nanocarriers were then pelleted down by centrifugation (15000 rpm, 15 min) and the supernatant was discarded. The pellet was washed with hexane repeatedly. Next, the nanocarriers were dispersed in 10 mL water, dialyzed against water, freeze dried, and stored at −20° C. until use.

### Example 6

### Adenosine Aids in Age-Related Bone Healing

### Introduction

The elderly population suffers more from bone fractures and subsequent delayed healing or even non-healing. It was verified whether aged mouse bone marrow cells are responsive to extracellular adenosine treatment toward osteogenic differentiation and scaffold assisted adenosine delivery can promote bone healing with age.

### Materials and Methods

Measurement of Alizarin Red S Deposition In Vitro from Both Young and Old Mice Bone Marrow Stromal Cells Treated with Adenosine Supplementation in Medium

Alizarin Red S, an anthraquinone derivative, may be used to identify calcium in tissue sections. The reaction is not strictly specific for calcium, since magnesium, manganese, barium, strontium, and iron may interfere, but these elements usually do not occur in sufficient concentration to interfere with the staining. Calcium forms an Alizarin Red S-calcium complex in a chelation process, and the end product is birefringent.

### Materials:

Cell sources:
Young mouse bone marrow (3-mo old)
Old mouse bone marrow (27-mo old)
Used at P2
Growth medium (GM):
αMEM, clear (Gibco 41061-029, 500 ML)
10% FBS (Gibco 16000-044, LOT1780016, 500 ML)
1% Pen Strep (Gibco 15140-122, 100 ML)
Osteogenic inducing medium (OM):
αMEM
10% FBS
10 mM β-glycerophosphate (Sigma, Cat #G9422)
50 mM ascorbic acid-2-phosphate (Sigma, Cat #A4403 or A8960)
100 nM dexamethasone (Sigma, Cat #D2915)
0.5% Pen Strep
Adenosine stock
6 mg/mL ADO [Sigma A4036-25G]
αMEM [Gibco 41061-029, 500 ML]
−20° C., dilute 100× (60 ug/mL) when use
GM with phosphate supplementation (3 mM):
Stock A:
53.4 mg sodium phosphate dibasic
50 mL GM
Stock B:
12 mg monobasic sodium phosphate
50 mL GM
3 mM (50 mL):
19.165 mL of A
19.165 mL of B
11.67 mL of GM
Alizarin Red Solution (2% w/v):
Alizarin Red S (Sigma-Aldrich, Cat #A5533) e.g. 1 g Alizarin Red S in 50 mL distilled water (plastic or glass container).
Filter with 40 um cell strainer.
Mix well. Adjust the pH to 4.1˜4.3 with ammonium hydroxide. The pH is critical, so make fresh or check pH if the solution is more than one month old.
Hydrochloric acid (0.5M)
Ammonium hydroxide (10% v/v, diluted 3× from 30%)

### Experimental Method:

Aspirate medium from cell dish (P1 cells).
Rinse with sterile PBS, aspirate.
Add 5 mL trypsin (T75 flask), incubate for 4 min at 37 C.
Add 5 mL GM to stop.
Use cell scraper to further release cells.
Transfer to tube, use additional 5 mL GM to rinse the dish and transfer.
Spin down at 1200 rpm for 4 min.
Resuspend cells, seed into culture plate at 20,000 cells/cm².
2 mL/well in 6-well
0.5 mL/well in 24-well
Freeze the rest cells in freezing medium.
10% DMSO
90% GM
Prepare GM-ADO (60 ug/mL) fresh:
18 mL GM
180 uL ADO stock (6 mg/mL)
Refresh medium daily for two weeks with the culture conditions:
GM
GM-ADO
OM (positive control)
In the third week, use phosphate supplementation:
GM+ADO +3 mM phosphate
GM+3 mM phosphate
OM (positive control)
Alizarin red S staining at 14 d and 21 d
Wash cells gently with PBS.
Fix with 4% PFA for 15 min. Discard and wash with MilliQ water.
Cover and stain with the Alizarin Red Solution for 10 min, and observe the reaction. Usually 5 minutes will produce nice red-orange staining of calcium.
Very gently wash with milliQ water to rinse off random precipitates. Discard milliQ water.
Alizarin red S quantification
Add 500 ul of HCl (0.5M) to each well for 10 min. at room temp with gentle shaking.
Transfer HCl solution in each well into one 1.5 ml microcentrifuge tube. Vortex for 30 sec.
Heat at 85° C. for 10 min. (Make sure the lid does not open during heating by adding weight to top).
Transfer to ice for 5 min. (Do not open lid until fully cooled).
Centrifuge at 20,000 g for 15 min. and remove 500 uL of supernatant to a new 1.5-mL microcentrifuge tube.
Add 200 uL of 10% (v/v) ammonium hydroxide to neutralize the acid. Mix well.
Notice the dye turning color from yellow to purple.
Read 150 uL of the supernatant at 405 nm in a spectrophotometer in a 96-well using opaque-walled, transparent-bottomed plates.
Normalize data against female aged cells.

Both young and aged cells from either sex were responsive to adenosine treatment in 2D culture and induced osteogenic differentiation, suggesting modulation of adenosine signaling in aged cells can promote osteogenesis. A larger fold change of alizarin red S deposition was seen in female when comparing ADO to GM. Thus, adenosine delivery and treatment can rescue the osteogenic differentiation of aged bone marrow stromal cells.

### In Vivo Sequestration of Adenosine Through Injectable HA Microgel in Young Mice

### Materials:

HA microgels (5 mg)
HA/HA-PBA microgels (1:1, 5 mg)
HA-PBA microgels (5 mg)
Female mouse (×9), C57BL/6J, 12 wk old

### Experimental Method:

Tibial fracture was performed according to lab protocol.
HA microgels containing various amount of PBA (0, 50%, 100%) were injected at fracture site.
At 3d, mice sacrificed, materials recovered.
Rinse and homogenize for adenosine assay

Microgels containing PBA sequestered endogenous adenosine following fracture injury, in a manner proportional to PBA content and consistent with the PEG-PBA scaffold. Thus, in addition to adenosine delivery, PBA microgels can also leverage intrinsic extracellular adenosine in aged fracture healing.

Evaluation of Fracture Healing Outcome of Aged Mice with Intervention of Adenosine Laden Microgels

### Materials:

HA, HA-ADO 3 mg microgels
Old mice (76 wk, male, C57BL6/J)

### Experimental Method:

Tibial fracture was induced according to lab protocol.
21d post implantation, mice sacrificed, tissue processed for uCT and histological staining.

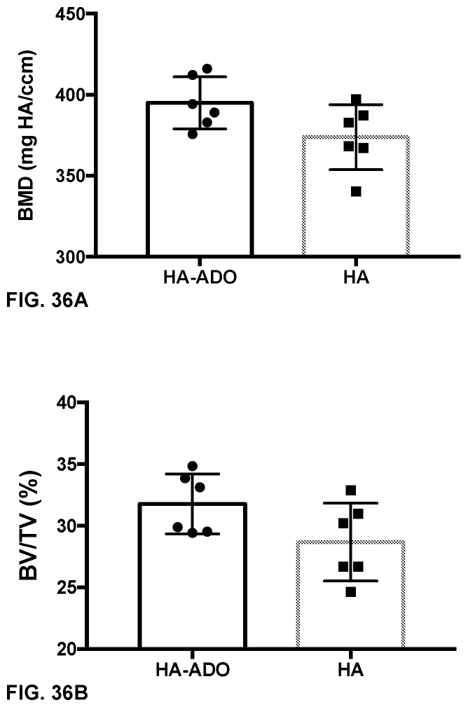

Microgels were less degradable than expected. With adenosine delivery, bone volume was slightly increased from uCT measurements, albeit not significant (FIGS. 36A-36B). In safranin O staining, healing was evidently delayed without intervention, as evidenced by remaining cartilage tissue in callus. In TRAP staining, instead, there was less osteoclastic activity in callus without intervention, showing lack of remodeling. Similarly, in endomucin IF staining, callus with intervention had more vessel ingrowth compared to that without adenosine intervention.

Thus, adenosine delivery by microgels improved fracture healing in aged mice. However, bone volume increase was not sufficient, and HA degradability in vivo seems to be less ideal, which can impede healing process.

### REFERENCES

Alam M., Costales M., Williams K., Down-regulation of CD73 expression favors host protection during Intracellular foodborne bacterial infections (IRC8P.496). J. Immunol. 192, 190.24 (2014).
Almeida M., Iyer S., Martin-Millan M., Bartell S. M., Han L., Ambrogini E., Onal M., Xiong J., Weinstein R. S., Jilka R. L., O'Brien C. A., Manolagas S. C., Estrogen receptor-α signaling in osteoblast progenitors stimulates cortical bone accrual. J. Clin. Invest. 123, 394-404 (2013).
Amin, S., Achenbach, S. J., Atkinson, E. J., Khosla, S. & Melton, L. J. Trends in Fracture Incidence: A Population-Based Study Over 20 Years. J Bone Miner Res 29, 581-589 (2014).
Antonioli, L., Blandizzi, C., Pacher, P. & Hasko, G. Immunity, inflammation and cancer: a leading role for adenosine. Nat Rev Cancer 13, 842-857, doi:10.1038/nrc3613 (2013).
Ayala, R. et al. Engineering the cell-material interface for controlling stem cell adhesion, migration, and differentiation. Biomaterials 32, 3700-3711, doi:10.1016/j.biomaterials.2011.02.004 (2011).
Baht, G. S., Nadesan, P., Silkstone, D. & Alman, B. A. Pharmacologically targeting beta-catenin for NF1 associated deficiencies in fracture repair. Bone 98, 31-36, doi:10.1016/j.bone.2017.02.012 (2017).
Biaggioni, I., Olafsson, B., Robertson, R. M., Hollister, A. S. & Robertson, D. Cardiovascular and respiratory effects of adenosine in conscious man. Evidence for chemoreceptor activation. Circulation research 61, 779-786 (1987).
Borowiak, M. et al. Small molecules efficiently direct endodermal differentiation of mouse and human embryonic stem cells. Cell Stem Cell 4, 348-358, doi:10.1016/j.stem.2009.01.014 (2009).
Bradaschia-Correa V., Josephson A. M., Egol A. J., Mizrahi M. M., Leclerc K., Huo J., Cronstein B. N., Leucht P., Ecto-5′-nucleotidase (CD73) regulates bone formation and remodeling during intramembranous bone repair in aging mice. Tissue Cell 49, 545-551 (2017).
Brooks, W. L. A. & Sumerlin, B. S. Synthesis and Applications of Boronic Acid-Containing Polymers: From Materials to Medicine. Chem. Rev. 116, 1375-1397, doi:10.1021/acs.chemrev.5b00300 (2016).
Brusatin, G., Panciera, T., Gandin, A., Citron, A. & Piccolo, S. Biomaterials and engineered microenvironments to control YAP/TAZ-dependent cell behaviour. Nat Mater 17, 1063-1075, doi:10.1038/s41563-018-0180-8 (2018).
Buchheiser A., Ebner A., Burghoff S., Ding Z., Romio M., Viethen C., Lindecke A., Köhrer K., Fisher J. W., Schrader J., Inactivation of CD73 promotes atherogenesis in apolipoprotein E-deficient mice. Cardiovasc. Res. 92, 338-347 (2011).
Burdick, J. A.; Chung, C.; Jia, X.; Randolph, M. A.; Langer, R., Controlled degradation and mechanical behavior of photopolymerized hyaluronic acid networks. Biomacromolecules 2005, 6 (1), 386-91.
Burdick, Jason A., Mauck, Robert L. & Gerecht, S. To Serve and Protect: Hydrogels to Improve Stem Cell-Based Therapies. Cell Stem Cell 18, 13-15, doi:10.1016/j.stem.2015.12.004 (2016).
Burge R., Dawson-Hughes B., Solomon D. H., Wong J. B., King A., Tosteson A., Incidence and economic burden of osteoporosis-related fractures in the United States, 2005-2025. J. Bone Miner. Res. 22, 465-475 (2007).
Burge, R. et al. Incidence and economic burden of osteoporosis-related fractures in the United States, 2005-2025. J Bone Miner Res 22, 465-475, doi:10.1359/jbmr.061113 (2007).
Carroll S. H., Wigner N. A., Kulkarni N., Johnston-Cox H., Gerstenfeld L. C., Ravid K., A2B adenosine receptor promotes mesenchymal stem cell differentiation to osteoblasts and bone formation in vivo. J. Biol. Chem. 287, 15718-15727 (2012).
Carroll, S. H. & Ravid, K. Differentiation of mesenchymal stem cells to osteoblasts and chondrocytes: a focus on adenosine receptors. Expert Reviews in Molecular Medicine 15, doi:10.1017/erm.2013.2 (2013).
Cenci S., Toraldo G., Weitzmann M. N., Roggia C., Gao Y., Qian W. P., Sierra O., Pacifici R., Estrogen deficiency induces bone loss by increasing T cell proliferation and lifespan through IFN-γ-induced class II transactivator. Proc. Natl. Acad. Sci. U.S.A. 100, 10405-10410 (2003).
Chang J., Wang Z., Tang E., Fan Z., McCauley L., Franceschi R., Guan K., Krebsbach P. H., Wang C.-Y., Inhibition of osteoblastic bone formation by nuclear factor-κB. Nat. Med. 15, 682-689 (2009).
Chen, F. M., Wu, L. A., Zhang, M., Zhang, R. & Sun, H. H. Homing of endogenous stem/progenitor cells for in situ tissue regeneration: Promises, strategies, and translational perspectives. Biomaterials 32, 3189-3209 (2011).
Chen, S. Y.; Yu, H. T.; Kao, J. P.; Yang, C. C.; Chiang, S. S.; Mishchuk, D. O.; Mau, J. L.; Slupsky, C. M., An NMR Metabolomic Study on the Effect of Alendronate in Ovariectomized Mice. Plos One 2014, 9 (9).
Cheng, H.; Chawla, A.; Yang, Y.; Li, Y.; Zhang, J.; Jang, H. L.; Khademhosseini, A., Development of nanomaterials for bone-targeted drug delivery. Drug Discov Today 2017, 22 (9), 1336-1350.
Chrobak P., Charlebois R., Rejtar P., El Bikai R., Allard B., Stagg J., CD73 plays a protective role in collagen-induced arthritis. J. Immunol. 194, 2487-2492 (2015).
Corciulo C., Wilder T., Cronstein B. N., Adenosine A2B receptors play an important role in bone homeostasis. Purinergic Signal 12, 537-547 (2016).
Corral, D. A.; Amling, M.; Priemel, M.; Loyer, E.; Fuchs, S.; Ducy, P.; Baron, R.; Karsenty, G., Dissociation between bone resorption and bone formation in osteopenic transgenic mice. P Natl Acad Sci USA 1998, 95 (23), 13835-13840.
Cronstein B. N., Adenosine, an endogenous anti-inflammatory agent. J. Appl. Physiol. 76, 5-13 (1994).
Cronstein, B. N. & Sitkovsky, M. Adenosine and adenosine receptors in the pathogenesis and treatment of rheumatic diseases. Nature Reviews Rheumatology 13, nrrheum.2016.2178, doi:10.1038/nrrheum.2016.178 (2017).
Deaglio S., Dwyer K. M., Gao W., Friedman D., Usheva A., Erat A., Chen J.-F., Enjyoji K., Linden J., Oukka M., Kuchroo V. K., Strom T. B., Robson S. C., Adenosine generation catalyzed by CD39 and CD73 expressed on regulatory T cells mediates immune suppression. J. Exp. Med. 204, 1257-1265 (2007).
Dimmeler, S., Ding, S., Rando, T. A. & Trounson, A. Translational strategies and challenges in regenerative medicine. Nat Med 20, 814-821 (2014).
Dwyer K. M., Deaglio S., Gao W., Friedman D., Strom T. B., Robson S. C., CD39 and control of cellular immune responses. Purinergic Signal 3, 171-180 (2007).
Eastell R., O'Neill T. W., Hofbauer L. C., Langdahl B., Reid I. R., Gold D. T., Cummings S. R., Postmenopausal osteoporosis. Nat. Rev. Dis. Primers 2, 16069 (2016).
Eghbali-Fatourechi G., Khosla S., Sanyal A., Boyle W. J., Lacey D. L., Riggs B. L., Role of RANK ligand in mediating increased bone resorption in early postmenopausal women. J. Clin. Invest. 111, 1221-1230 (2003).
Einhorn, T. A. & Gerstenfeld, L. C. Fracture healing: mechanisms and interventions. Nature Reviews Rheumatology 11, 45-54, doi:10.1038/nrrheum.2014.164 (2015).
Falahati-Nini A., Riggs B. L., Atkinson E. J., O'Fallon W. M., Eastell R., Khosla S., Relative contributions of testosterone and estrogen in regulating bone resorption and formation in normal elderly men. J. Clin. Invest. 106, 1553-1560 (2000).
Fletcher J. M., Lonergan R., Costelloe L., Kinsella K., Moran B., O'Farrelly C., Tubridy N., Mills K. H., CD39+Foxp3+ regulatory T cells suppress pathogenic Th17 cells and are impaired in multiple sclerosis. J. Immunol. 183, 7602-7610 (2009).
Fredholm, B. Adenosine, an endogenous distress signal, modulates tissue damage and repair. Cell death and differentiation 14, 1315 (2007).
Gaudin, A.; Yemisci, M.; Eroglu, H.; Lepetre-Mouelhi, S.; Turkoglu, O. F.; Donmez-Demir, B.; Caban, S.; Sargon, M. F.; Garcia-Argote, S.; Pieters, G.; Loreau, O.; Rousseau, B.; Tagit, O.; Hildebrandt, N.; Le Dantec, Y.; Mougin, J.; Valetti, S.; Chacun, H.; Nicolas, V.; Desmaele, D.; Andrieux, K.; Capan, Y.; Dalkara, T.; Couvreur, P., Squalenoyl adenosine nanoparticles provide neuroprotection after stroke and spinal cord injury. Nat Nanotechnol 2014, 9 (12), 1054-1062.
Gharibi B., Abraham A. A., Ham J., Evans B. A., Adenosine receptor subtype expression and activation influence the differentiation of mesenchymal stem cells to osteoblasts and adipocytes. J. Bone Miner. Res. 26, 2112-2124 (2011).
Gonzalez Diaz, E., Shih, Y., Nakasaki, M., Liu, M. & Varghese, S. Mineralized biomaterials mediated repair of bone defects through endogenous cells. Tissue Engineering (2018).
Grayson, W. L. et al. Stromal cells and stem cells in clinical bone regeneration. Nat Rev Endocrinol 11, 140-150, doi:10.1038/nrendo.2014.234 (2015).
Haffner-Luntzer, M., Kovtun, A., Rapp, A. E. & Ignatius, A. Mouse models in bone fracture healing research. Curr Mol Bio Rep 2, 101-111 (2016).
Ham, J. & Evans, B. A. An emerging role for adenosine and its receptors in bone homeostasis. Front Endocrinol (Lausanne) 3, 113, doi:10.3389/fendo.2012.00113 (2012).
Hart M. L., Henn M., Köhler D., Kloor D., Mittelbronn M., Gorzolla I. C., Stahl G. L., Eltzschig H. K., Role of extracellular nucleotide phosphohydrolysis in intestinal ischemia-reperfusion injury. FASEB J. 22, 2784-2797 (2008).
Haskó, G., Linden, J., Cronstein, B. & Pacher, P. Adenosine receptors: therapeutic aspects for inflammatory and immune diseases. Nature reviews Drug discovery 7, 759 (2008).
Hawse J. R., Subramaniam M., Ingle J. N., Oursler M. J., Raj amannan N. M., Spelsberg T. C., Estrogen-TGFβ cross-talk in bone and other cell types: Role of TIEG, Runx2, and other transcription factors. J. Cell. Biochem. 103, 383-392 (2008).
He W. J., Wilder T., Cronstein B. N., Rolofylline, an adenosine A1 receptor antagonist, inhibits osteoclast differentiation as an inverse agonist. Br. J. Pharmacol. 170, 1167-1176 (2013).
He W., Cronstein B. N., Adenosine A1 receptor regulates osteoclast formation by altering TRAF6/TAK1 signaling. Purinergic Signal 8, 327-337 (2012).
He W., Mazumder A., Wilder T., Cronstein B. N., Adenosine regulates bone metabolism via A1, A2A, and A2B receptors in bone marrow cells from normal humans and patients with multiple myeloma. FASEB J. 27, 3446-3454 (2013).
Heller, D. A.; Levi, Y.; Pelet, J. M.; Doloff, J. C.; Wallas, J.; Pratt, G. W.; Jiang, S.; Sahay, G.; Schroeder, A.; Schroeder, J. E.; Chyan, Y.; Zurenko, C.; Querbes, W.; Manzano, M.; Kohane, D. S.; Langer, R.; Anderson, D. G., Modular ‘Click-in-Emulsion’ Bone-Targeted Nanogels. Advanced Materials 2013, 25 (10), 1449-1454.
Hinz S., Lacher S. K., Seibt B. F., Müller C. E., BAY60-6583 acts as a partial agonist at adenosine A2B receptors. J. Pharmacol. Exp. Ther. 349, 427-436 (2014).
Horenstein A. L., Chillemi A., Zaccarello G., Bruzzone S., Quarona V Zito A Serra S., Malavasi F., A CD38/CD203a/CD73 ectoenzymatic pathway independent of CD39 drives a novel adenosinergic loop in human T lymphocytes. Oncoimmunology 2, e26246 (2013).
Hu, C. M.; Fang, R. H.; Wang, K. C.; Luk, B. T.; Thamphiwatana, S.; Dehaini, D.; Nguyen, P.; Angsantikul, P.; Wen, C. H.; Kroll, A. V.; Carpenter, C.; Ramesh, M.; Qu, V.; Patel, S. H.; Zhu, J.; Shi, W.; Hofman, F. M.; Chen, T. C.; Gao, W.; Zhang, K.; Chien, S.; Zhang, L., Nanoparticle biointerfacing by platelet membrane cloaking. Nature 2015, 526 (7571), 118-21.
Huangfu, D. et al. Induction of pluripotent stem cells by defined factors is greatly improved by small-molecule compounds. Nat Biotech 26, 795 (2008).
Hwang, Y., Zhang, C. & Varghese, S. Poly(ethylene glycol) cryogels as potential cell scaffolds: effect of polymerization conditions on cryogel microstructure and properties. J. Mater. Chem. 20, 345-351, doi:10.1039/B917142H (2010).
Idris A. I., Ovariectomy/orchidectomy in rodents. Methods Mol. Biol. 816, 545-551 (2012).
Jarvinen, T. A.; Ruoslahti, E., Target-seeking antifibrotic compound enhances wound healing and suppresses scar formation in mice. Proc Natl Acad Sci USA 2010, 107 (50), 21671-6.
Kalu D. N., Chen C., Ovariectomized murine model of postmenopausal calcium malabsorption. J. Bone Miner. Res. 14, 593-601 (1999).
Kang H., Shih Y.-R., Varghese S., Biomineralized matrices dominate soluble cues to direct osteogenic differentiation of human mesenchymal stem cells through adenosine signaling. Biomacromolecules 16, 1050-1061 (2015).
Kang, H. et al. Biomineralized matrix-assisted osteogenic differentiation of human embryonic stem cells. J. Mater. Chem. B 2, 5676-5688, doi:10.1039/C4TB00714J (2014).
Kang, H. et al. Mineralized gelatin methacrylate-based matrices induce osteogenic differentiation of human induced pluripotent stem cells. Acta Biomaterialia 10, 4961-4970, doi:10.1016/j.actbio.2014.08.010 (2014).
Kang, H., Shih, Y.-R. V., Nakasaki, M., Kabra, H. & Varghese, S. Small molecule-driven direct conversion of human pluripotent stem cells into functional osteoblasts. Science Advances 2, e1600691, doi:10.1126/sciadv.1600691 (2016).
Kang, H., Zeng, Y. & Varghese, S. Functionally graded multilayer scaffolds for in vivo osteochondral tissue engineering. Acta Biomater 78, 365-377, doi:10.1016/j.actbio.2018.07.039 (2018).
Kanthi Y., Hyman M. C., Liao H., Baelc A. E., Visovatti S. H., Sutton N. R., Goonewardena S. N., Neral M. K., Jo H., Pinsky D. J., Flow-dependent expression of ectonucleotide tri(di) phosphohydrolase-1 and suppression of atherosclerosis. J. Clin. Invest. 125, 3027-3036 (2015).
Kara F. M., Doty S. B., Boskey A., Goldring S., Zaidi M., Fredholm B. B., Cronstein B. N., Adenosine A1 receptors regulate bone resorption in mice: Adenosine A1 receptor blockade or deletion increases bone density and prevents ovariectomy-induced bone loss in adenosine A1 receptor-knockout mice. Arthritis Rheum. 62, 534-541 (2010).
Katebi M., Soleimani M., Cronstein B. N., Adenosine A2A receptors play an active role in mouse bone marrow-derived mesenchymal stem cell development. J. Leukoc. Biol. 85, 438-444 (2009).
Kazemzadeh-Narbat, M. et al. Adenosine-associated delivery systems. J Drug Target 23, 580-596, doi:10.3109/1061186X.2015.1058803 (2015).
Khademhosseini, A. & Langer, R. A decade of progress in tissue engineering. Nature Protocols 11, 1775-1781, doi:10.1038/nprot.2016.123 (2016).
Köhler D., Eckle T., Faigle M., Grenz A., Mittelbronn M., Laucher S., Hart M. L., Robson S. C., Müller C. E., Eltzschig H. K., CD39/ectonucleoside triphosphate diphosphohydrolase 1 provides myocardial protection during cardiac ischemia/reperfusion injury. Circulation 116, 1784-1794 (2007).
Kusumbe, A. P., Ramasamy, S. K. & Adams, R. H. Coupling of angiogenesis and osteogenesis by a specific vessel subtype in bone. Nature 507, 323-328, doi:10.1038/nature13145 (2014).
Lee, K., Silva, E. A. & Mooney, D. J. Growth factor delivery-based tissue engineering:

general approaches and a review of recent developments. J R Soc Interface 8, 153-170 (2010).
Li Q., Price T. P., Sundberg J. P., Uitto J., Juxta-articular joint-capsule mineralization in CD73 deficient mice: Similarities to patients with NTSE mutations. Cell Cycle 13, 2609-2615 (2014).
Lopez, C. D. et al. Local delivery of adenosine receptor agonists to promote bone regeneration and defect healing. Adv Drug Deliv Rev, doi:10.1016/j.addr.2018.06.010 (2018).
Manolagas S. C., From estrogen-centric to aging and oxidative stress: A revised perspective of the pathogenesis of osteoporosis. Endocr. Rev. 31, 266-300 (2010).
Mediero A., Kara F. M., Wilder T., Cronstein B. N., Adenosine A2A receptor ligation inhibits osteoclast formation. Am. J. Pathol. 180, 775-786 (2012).
Mediero A., Perez-Aso M., Cronstein B. N., Activation of adenosine A2A receptor reduces osteoclast formation via PKA- and ERK1/2-mediated suppression of NFκB nuclear translocation. Br. J. Pharmacol. 169, 1372-1388 (2013).
Mediero A., Wilder T., Reddy V. S. R., Cheng Q., Tovar N., Coelho P. G., Witek L., Whatling C., Cronstein B. N., Ticagrelor regulates osteoblast and osteoclast function and promotes bone formation in vivo via an adenosine-dependent mechanism. FASEB J. 30, 3887-3900 (2016).
Mediero A., Wilder T., Shah L., Cronstein B. N., Adenosine A2A receptor (A2AR) stimulation modulates expression of semaphorins 4D and 3A, regulators of bone homeostasis. FASEB J. 32, 3487-3501 (2018).
Mediero, A., Wilder, T., Perez-Aso, M. & Cronstein, B. N. Direct or indirect stimulation of adenosine A2A receptors enhances bone regeneration as well as bone morphogenetic protein-2. FASEB J 29, 1577-1590, doi:10.1096414-265066 (2015).
Mediero, A.; Cronstein, B. N., Adenosine and bone metabolism. Trends Endocrinol Metab 2013, 24 (6), 290-300.
Meling, T. R. et al. Adenosine-assisted clipping of intracranial aneurysms. Neurosurg Rev 41, 585-592, doi:10.1007/s10143-017-0896-y (2018).
Montesinos, M. C. et al. Adenosine promotes wound healing and mediates angiogenesis in response to tissue injury via occupancy of A(2A) receptors. Am J Pathol 160, 2009-2018, doi:10.1016/S0002-9440(10)61151-0 (2002).
Morandi F., Horenstein A. L., Chillemi A., Quarona V., Chiesa S., Imperatori A., Zanellato S., Mortara L., Gattorno M., Pistoia V., Malavasi F., CD56brightCD16-NK cells produce adenosine through a CD38-mediated pathway and act as regulatory cells inhibiting autologous CD4+ T Cell proliferation. J. Immunol. 195, 965-972 (2015).
Mundy G. R., Osteoporosis and inflammation. Nutr. Rev. 65, S147-S151 (2007).
Nakamura T., Imai Y., Matsumoto T., Sato S., Takeuchi K., Igarashi K., Harada Y., Azuma Y., Krust A., Yamamoto Y., Nishina H., Takeda S., Takayanagi H., Metzger D., Kanno J., Takaoka K., Martin T. J., Chambon P., Kato S., Estrogen prevents bone loss via estrogen receptor α and induction of Fas ligand in osteoclasts. Cell 130, 811-823 (2007).
Pastor-Anglada M., Pérez-Torras S., Who is who in adenosine transport. Front. Pharmacol. 9, 627 (2018).
Phinney, D. G. & Pittenger, M. F. Concise Review: MSC-Derived Exosomes for Cell-Free Therapy. Stem Cells 35, 851-858, doi:10.1002/stem.2575 (2017).
Raczkowski F., Rissiek A., Ricklefs I., Heiss K., Schumacher V., Wundenberg K., Haag F., Koch-Nolte F., Tolosa E., Mittrucker H.-W., CD39 is upregulated during activation of mouse and human T cells and attenuates the immune response to Listeria monocytogenes. PLOS ONE 13, e0197151 (2018).
Raemdonck, K.; Naeye, B.; Buyens, K.; Vandenbroucke, R. E.; Hogset, A.; Demeester, J.; De Smedt, S. C., Biodegradable Dextran Nanogels for RNA Interference: Focusing on Endosomal Escape and Intracellular siRNA Delivery. Adv Funct Mater 2009, 19 (9), 1406-1415.
Riggs B. L., The mechanisms of estrogen regulation of bone resorption. J. Clin. Invest. 106, 1203-1204 (2000).
Rosales, A. M. & Anseth, K. S. The design of reversible hydrogels to capture extracellular matrix dynamics. Nature Reviews Materials 1, 15012, doi:10.1038/natrevmats.2015.12 (2016).
Rosenblum, D.; Joshi, N.; Tao, W.; Karp, J. M.; Peer, D., Progress and challenges towards targeted delivery of cancer therapeutics. Nat Commun 2018, 9 (1), 1410.
Roszek, K. & Wujak, M. How to influence the mesenchymal stem cells fate? Emerging role of ectoenzymes metabolizing nucleotides. J. Cell. Physiol. 234, 320-334 (2019).
Rotman, S. G.; Grijpma, D. W.; Richards, R. G.; Moriarty, T. F.; Eglin, D.; Guillaume, O., Drug delivery systems functionalized with bone mineral seeking agents for bone targeted therapeutics. J Control Release 2018, 269, 88-99.
Ryu, J. H., Lee, G. J., Shih, Y., Kim, T. & Varghese, S. Phenylboronic acid-polymers for biomedical applications. Current medicinal chemistry (2018).
Salem, H. K. & Thiemermann, C. Mesenchymal stromal cells: current understanding and clinical status. Stem Cells 28, 585-596, doi:10.1002/stem.269 (2010).
Sauer A. V., Brigida I., Carriglio N., Aiuti A., Autoimmune dysregulation and purine metabolism in adenosine deaminase deficiency. Front. Immunol. 3, 265 (2012).
Seale, N. M. & Varghese, S. Biomaterials for pluripotent stem cell engineering: from fate determination to vascularization. J. Mater. Chem. B, doi:10.1039/C5TB02658J (2016).
Segers, V. F. M. & Lee, R. T. Stem-cell therapy for cardiac disease. Nature 451, 937-942, doi:10.1038/nature06800 (2008).
Shevde N. K., Bendixen A. C., Dienger K. M., Pike J. W., Estrogens suppress RANK ligand-induced osteoclast differentiation via a stromal cell independent mechanism involving c-Jun repression. Proc. Natl. Acad. Sci. U.S.A. 97, 7829-7834 (2000).
Shih Y.-R., Hwang Y., Phadke A., Kang H., Hwang N. S., Caro E. J., Nguyen S., Siu M., Theodorakis E. A., Gianneschi N. C., Vecchio K. S., Chien S., Lee O. K., Varghese S., Calcium phosphate-bearing matrices induce osteogenic differentiation of stem cells through adenosine signaling. Proc. Natl. Acad. Sci. U.S.A. 111, 990-995 (2014).
Shih Y.-R., Kang H., Rao V., Chiu Y.-J., Kwon S. K., Varghese S., In vivo engineering of bone tissues with hematopoietic functions and mixed chimerism. Proc. Natl. Acad. Sci. U.S.A. 114, 5419-5424 (2017).
Shih, Y. V.; Liu, M.; Kwon, S. K.; Lida, M.; Gong, Y.; Sangaj, N.; Varghese, S., Dysregulation of ectonucleotidase-mediated extracellular adenosine during postmenopausal bone loss. Sci Adv 2019, 5 (8), eaax1387.
Shih, Y.-R. et al. Synthetic bone mimetic matrix-mediated in situ bone tissue formation through host cell recruitment. Acta Biomaterialia 19, 1-9, doi:10.1016/j.actbio.2015.03.017 (2015).
Smith E. P., Boyd J., Frank G. R., Takahashi H., Cohen R. M., Specker B., Williams T. C., Lubahn D. B., Korach K. S., Estrogen resistance caused by a mutation in the estrogen-receptor gene in a man. N. Engl. J. Med. 331, 1056-1061 (1994).
Sun X. F., Wu Y., Gao W. D., Enjyoji K., Csizmadia E., Muller C. E., Murakami T., Robson S. C., CD39/ENTPD1 Expression by CD4+Foxp3+ regulatory T cells promotes hepatic metastatic tumor growth in mice. Gastroenterology 139, 1030-1040 (2010).
Sun, Y.; Ye, X.; Cai, M.; Liu, X.; Xiao, J.; Zhang, C.; Wang, Y.; Yang, L.; Liu, J.; Li, S.; Kang, C.; Zhang, B.; Zhang, Q.; Wang, Z.; Hong, A.; Wang, X., Osteoblast-Targeting-Peptide Modified Nanoparticle for siRNA/microRNA Delivery. ACS Nano 2016, 10 (6), 5759-68.
Tai N., Wong F. S., Wen L., TLR9 deficiency promotes CD73 expression in T cells and diabetes protection in nonobese diabetic mice. J. Immunol. 191, 2926-2937 (2013).
Takahama, H.; Minamino, T.; Asanuma, H.; Fujita, M.; Asai, T.; Wakeno, M.; Sasaki, H.; Kikuchi, H.; Hashimoto, K.; Oku, N.; Asakura, M.; Kim, J.; Takashima, S.; Komamura, K.; Sugimachi, M.; Mochizuki, N.; Kitakaze, M., Prolonged targeting of ischemic/reperfused myocardium by liposomal adenosine augments cardioprotection in rats. J Am Coll Cardiol 2009, 53 (8), 709-17.
Takedachi M., Oohara H., Smith B. J., Iyama M., Kobashi M., Maeda K., Long C. L., Humphrey M. B., Stoecker B. J., Toyosawa S., Thompson L. F., Murakami S., CD73-generated adenosine promotes osteoblast differentiation. J. Cell. Physiol. 227, 2622-2631 (2012).
U.S. Office of the Surgeon General, Bone Health and Osteoporosis: A Report of the Surgeon General (United States Public Health Service, 2004).
Vanderburgh, J.; Hill, J. L.; Gupta, M. K.; Kwakwa, K. A.; Wang, S. K.; Moyer, K.; Bedingfield, S. K.; Merkel, A. R.; d′Arcy, R.; Guelcher, S. A.; Rhoades, J. A.; Duvall, C. L., Tuning Ligand Density To Optimize Pharmacokinetics of Targeted Nanoparticles for Dual Protection against Tumor-Induced Bone Destruction. ACS Nano 2020, 14 (1), 311-327.
Varghese, S. et al. Engineering musculoskeletal tissues with human embryonic germ cell derivatives. Stem cells 28, 765-774 (2010).
Vijayan D., Young A., Teng M. W. L., Smyth M. J., Targeting immunosuppressive adenosine in cancer. Nat. Rev. Cancer 17, 709-724 (2017).
Webber, M. J., Appel, E. A., Meijer, E. W. & Langer, R. Supramolecular biomaterials. Nature Materials 15, 13-26, doi:10.1038/nmat4474 (2016).
Weitzmann M. N., Pacifici R., Estrogen deficiency and bone loss: An inflammatory tale. J. Clin. Invest. 116, 1186-1194 (2006).
Weitzmann M. N., Roggia C., Toraldo G., Weitzmann L., Pacifici R., Increased production of IL-7 uncouples bone formation from bone resorption during estrogen deficiency. J. Clin. Invest. 110, 1643-1650 (2002).
Xu, R. et al. Targeting skeletal endothelium to ameliorate bone loss. Nat Med 24, 823-833, doi:10.1038/s41591-018-0020-z (2018).
Yang, Y. S.; Xie, J.; Wang, D.; Kim, J. M.; Tai, P. W. L.; Gravallese, E.; Gao, G.; Shim, J. H., Bone-targeting AAV-mediated silencing of Schnurri-3 prevents bone loss in osteoporosis. Nat Commun 2019, 10 (1), 2958.
Yegutkin G. G., Enzymes involved in metabolism of extracellular nucleotides and nucleosides: Functional implications and measurement of activities. Crit. Rev. Biochem. Mol. Biol. 49, 473-497 (2014).
Yegutkin G. G., Nucleotide- and nucleoside-converting ectoenzymes: Important modulators of purinergic signalling cascade. Biochim. Biophys. Acta 1783, 673-694 (2008).
Yin, Q.; Tang, L.; Cai, K.; Tong, R.; Sternberg, R.; Yang, X.; Dobrucki, L. W.; Borst, L. B.; Kamstock, D.; Song, Z.; Helferich, W. G.; Cheng, J.; Fan, T. M., Pamidronate functionalized nanoconjugates for targeted therapy of focal skeletal malignant osteolysis. Proc Natl Acad Sci U S A 2016, 113 (32), E4601-9.
Zeng, Y.; Hogue, J.; Varghese, S., Biomaterial-assisted local and systemic delivery of bioactive agents for bone repair. Acta Biomater 2019.
Zeng, Y.; Shih, Y. V.; Baht, G. S.; Varghese, S., In Vivo Sequestration of Innate Small Molecules to Promote Bone Healing. Adv Mater 2019, e1906022.
Zhang, S.; Gangal, G.; Uludag, H., ‘Magic bullets’ for bone diseases: progress in rational design of bone-seeking medicinal agents. Chem Soc Rev 2007, 36 (3), 507-31.
Zhou S., Zilberman Y., Wassermann K., Bain S. D., Sadovsky Y., Gazit D., Estrogen modulates estrogen receptor α and β expression, osteogenic activity, and apoptosis in mesenchymal stem cells (MSCs) of osteoporotic mice. J. Cell. Biochem. 81 (suppl. 36), 144-155 (2001).
Zhou, Z. et al. Reversible Covalent Cross-Linked Polycations with Enhanced Stability and ATP-Responsive Behavior for Improved siRNA Delivery. Biomacromolecules, doi:10.1021/acs.biomac.8b00922 (2018).

We claim:

1. A biomaterial comprising a polymer and a bioactive molecule binding moiety.

2. The biomaterial of claim 1 wherein the polymer is hyaluronic acid (HA), 2-(methacryloyloxy)ethyl acetoacetate (2MAEA), or polyethylene glycol (PEG).

3. The biomaterial of claim 2 wherein the polymer is HA.

4. The biomaterial of any one of claims 1-3 wherein the bioactive molecule binding moiety is an osteoanabolic molecule binding moiety.

5. The biomaterial of claim 4 wherein the osteoanabolic molecule binding moiety is a boronate molecule.

6. The biomaterial of claim 5 wherein the boronate molecule is phenylboronic acid (PBA).

7. The biomaterial of claim 4 wherein the osteoanabolic molecule binding moiety is a ketal group.

8. The biomaterial of any one of claims 1-7 further comprising a bone targeting moiety.

9. The biomaterial of claim 8 wherein the bone targeting moiety is a bisphosphonate molecule.

10. The biomaterial of claim 9 wherein the bisphosphonate molecule is alendronate.

11. The biomaterial of any one of claims 1-10 further comprising a bioactive molecule.

12. The biomaterial of claim 11 wherein the bioactive molecule is an osteoanabolic molecule.

13. The biomaterial of claim 12 wherein the osteoanabolic molecule is an adenosine compound or an Adenosine A2B receptor (A2BR) agonist.

14. The biomaterial of claim 13 wherein the adenosine compound is adenosine or polyadenosine.

15. The biomaterial of any one of claims 12-14 wherein the osteoanabolic molecule is adenosine.

16. The biomaterial of any one of claims 1-15 wherein the biomaterial is formulated for systemic delivery.

17. The biomaterial of any one of claims 1-15 wherein the biomaterial is formulated for local delivery.

18. The biomaterial of any one of claims 1-15 wherein the biomaterial is formulated as a hydrogel, a nanogel, a microgel, a tablet, a patch, a coating for an orthopedic implant, an ointment, a cream, or a scaffold.

19. The microgel of claim 18 wherein the microgel has a diameter of 1-200 μm.

20. A pharmaceutical composition comprising the biomaterial of any one of claims 1-19 and a pharmaceutically acceptable carrier and/or excipient.

21. A method of reducing bone degeneration and/or promoting bone regeneration in a subject in need thereof comprising administering to the subject the biomaterial of any one of claims 1-19.

22. A method of promoting osteoblastogenesis and/or decreasing osteoclastogenesis in a subject in need thereof comprising administering to the subject the biomaterial of any one of claims 1-19.

23. A method of treating and/or preventing a low bone mass condition in a subject in need thereof comprising administering to the subject the biomaterial of any one of claims 1-19.

24. The method of claim 23 wherein the low bone mass condition is osteoporosis.

25. The method of claim 23 wherein the low bone mass condition is osteopenia.

26. A method of promoting bone fracture healing in a subject in need thereof comprising administering to the subject the biomaterial of any one of claims 1-19.

27. A method of repairing a skeletal defect in a subject in need thereof comprising administering to the subject the biomaterial of any one of claims 1-19.

28. A method of enhancing the innate ability of bone repair tissue to repair bone in a subject in need thereof comprising administering to the subject the biomaterial of any one of claims 1-19.

29. A method of activating A2BR to promote bone repair in a subject in need thereof comprising administering to the subject the biomaterial of any one of claims 1-19.

30. A method of enhancing the outcome of orthopedic implant surgery in a subject in need thereof comprising administering to the subject the biomaterial of any one of claims 1-19.

31. The method according to any one of claims 21-29 wherein the biomaterial is administered systemically.

32. The method according to claim 27 wherein the biomaterial is administered intravenously.

33. The method according to claim 30 wherein the orthopedic implant is coated with the biomaterial.

34. The method according to any one of claims 26-27 wherein the biomaterial is administered locally.

35. The method of claim 30 wherein the local administration is by injection of the biomaterial at the site of the bone injury.

36. The method of claim 30 wherein the local administration is by implantation of a patch or scaffold comprising the biomaterial.
